(19) United States
(12) Patent Application Publication
(10) Pub. No.: US 2011/0289632 A1
(43) Pub. Date: Nov. 24, 2011

(54) METHYLKETONE SYNTHASE, PRODUCTION OF METHYLKETONES IN PLANTS AND BACTERIA

(75) Inventors: Eran Pichersky, Chelsea, MI (US); Eyal Fridman, Ann Arbor, MI (US); Geng Yu, Ann Arbor, MI (US); Thuong T.H. Nguyen, Ann Arbor, MI (US); Joseph P. Noel, San Diego, CA (US); Imri Ben-Israel, Gealya (IL)

(73) Assignees: The Regents of the University of Michigan, Ann Arbor, MI (US); Salk Institute for Biological Studies, La Jolla, CA (US); Yissum Research Development Company of the Hebrew University of Jerusalem, Jerusalem (IL)

(21) Appl. No.: 12/888,849

(22) Filed: Sep. 23, 2010

Related U.S. Application Data
(60) Provisional application No. 61/245,905, filed on Sep. 25, 2009.

(51) Int. Cl. A01H 5/00; C12N 15/63; C07H 21/04; C12N 5/10; C12P 7/40; C12P 7/26; C12N 9/16; C12N 1/00

(52) U.S. Cl. 800/298; 435/196; 435/320.1; 435/243; 435/419; 435/136; 435/148; 536/23.2

(57) ABSTRACT

Isolated genes and amino acid sequences encode methylketone synthase 2 (MKS2) enzymes from tomato plants, including, ShMKS2 and SlMKS2. When expressed recombinantly in bacteria and other host cells, the MKS2 enzymes produce methylketones of various carbon chain lengths ranging from C₇ to C₂₀ from 3-ketoacyl intermediate substrates. Methylketones are known to have important roles in protecting plants against pests, and also as flavor compounds, and can be used as stockfeed in the chemical industry.

### CROSS-REFERENCE TO RELATED APPLICATIONS

This application claims the benefit of U.S. Provisional Application No. 61/245,905, filed on Sep. 25, 2009. The entire disclosure of the above application is incorporated herein by reference.

### GOVERNMENT RIGHTS

This invention was made with U.S. Government support by NRICG 2004-35318-14874 awarded by the U.S. Department of Agriculture and by EEC-0813570 awarded by the National Science Foundation. The Government has certain rights in the invention.

### FIELD

The present technology relates to methylketone synthase II (MKS2), an enzyme involved in the production of methylketone compounds from β-ketoacyl intermediates in the fatty acid biosynthetic pathway.

### INTRODUCTION

This section provides background information related to the present disclosure which is not necessarily prior art.

Plants synthesize a multitude of specialized compounds to help ward off pests. Some of these classes of compounds, like terpenes and phenolics, are widely distributed throughout the plant kingdom. Others occur sporadically or are limited to one or a few taxa. Medium-length methylketones (MK) are found throughout the plant kingdom, and include a class of compounds effective in protecting plants against pests. Studies on the composition of the essential oils of many species found medium-length methylketones in lime (Citrus limetta) leaves, in clove (Eugenia caryophyllus) and cinnamon (Cinnamomum zeylanicum) oil, in palm kernel (Lodoicea maldivica), peanut (Arachis hypogaea), cottonseed (Gossypium hirsutum), and sunflower (Helianthus annuus) seed oils, and in oil of hop (Humulus lupulus). 2-Tridecanone was characterized as a crystalline constituent of the essential oil of matsubasa (Shizandra nigra maxim), a plant in the magnolia family, which is used as a bath perfume. In some plants, the methylketone and the derived secondary alcohol are found together; for example, 2-heptanone and 2-heptanol in the oil of cloves.

Leaves of the wild tomato species Lycopersicon hirsutum f glabratum are among the most prominent sources of methylketones in plants. Several accessions of this wild species contain mainly the two methylketones 2-undecanone and 2-tridecanone, in concentrations ranging between 2700 and 5500 μg per g fresh weight. By comparison, the cultivated tomato L. esculentum has only minute amounts of these compounds, up to 80 μg per g. In one of the accessions of the L. hirsutum f glabratum (Genbank Accession No.: PI134417) the methylketones were reported to compose up to 90% of the tip contents of the glandular trichomes. Trichomes, both glandular and nonglandular, are prominent features of the foliage and stems in the genus Lycopersicon, with glandular trichomes predominating on most surfaces and the nonglandular trichomes predominating on leaf veins.

A methylketone synthase I enzyme exists in Solanum habrochaites f glabratum (formerly known as Lycopersicon hirsutum f glabratum), Accession Number P1126449). Gene identification was based on prevalence of specific sequences, comparisons with known sequences encoding enzymes of similar function (either in terms of substrates or type of reactions), metabolic profiling of the plant material, and enzymatic assays of candidate proteins; see Fridman and Pichersky, (2005) Curr. Opin. Plant Biol. 2005, 8(3):242-248, which is incorporated herein by reference. This approach allowed the determination that methylketones are made via the de novo fatty acid biosynthetic pathway in the chloroplast and identified MKS1 as an enzyme responsible for the reaction leading from C12, C14, and C16 β-ketoacyl-ACPs to the C11, C13, and C15 methylketones, respectively. The levels of MKS1 transcripts and protein are closely correlated with the presence of methylketones in the Lycopersicon genus. MKS1 is capable of both hydrolyzing the thioester bond and decarboxylating the resulting 3-ketoacid intermediate. However, it was noted that the turnover rate of the enzyme was unusually low.

Methylketones are important products and intermediates in the production of valuable chemicals, natural pesticides, and pharmaceuticals. For example, production of long chain tertiary amines can occur by reductive amination of C₁₀-C₂₆ alkyl ketones with secondary amines using a supported nickel catalyst. The tertiary amine products produced according to such methods can be used in various ways, including as fuel oil, stabilizers, and chemical intermediates. The tertiary amine products can also be economically converted to aliphatic amine oxides, which can be used as fabric softeners and conditioners. Additionally, they may be used as intermediates in the production of other chemicals.

Thus, there is a need for methylketones and means for synthesizing methylketones, and it would be highly advantageous to have specific enzymes capable of biologically synthesizing methylketones of various lengths to serve as intermediate compounds for the production of various industrial chemicals, and advantageous to have isolated enzymes capable of producing methylketones and polynucleotides encoding such enzymes.

### SUMMARY

This section provides a general summary of the disclosure, and is not a comprehensive disclosure of its full scope or all of its features.

The present technology provides isolated polypeptides and nucleic acids encoding methylketone synthase 2 (MKS2) from tomato and Arabidopsis, including for example, ShMKS2 and SlMKS2. Functional variants of MKS2 enzymes can produce methylketones of various carbon chain lengths ranging from C₇ to C₂₀. In some embodiments, an isolated polypeptide is provided that comprises an amino acid sequence that is at least 60%, 80%, 95%, or 100% identical to SlMKS2 (SEQ ID NO: 3) or ShMKS2 (SEQ ID NO: 4). Isolated polypeptides can also comprise various polypeptides, including transit peptides, which can include ShMKS2 (SEQ ID NO: 25), SlMKS2a (SEQ ID NO: 27), SlMKS2b (SEQ ID NO: 28), or SlMKS2c (SEQ ID NO: 26).

In some embodiments, isolated nucleic acids are provided that encode the various MKS2 polypeptides. For example, nucleic acids include those having a nucleotide sequence at least 60%, 80%, 95%, or 100% identical to a polynucleotide encoding the polypeptide SlMKS2 (SEQ ID NO: 3) or ShMKS2 (SEQ ID NO: 4). In some embodiments, the nucleic acid comprises a nucleotide sequence at least 60%, 80%, 95%, or 100% identical to a polynucleotide encoding various polypeptides, including transit peptides, which can include ShMKS2 (SEQ ID NO: 25), SlMKS2a (SEQ ID NO: 27), SlMKS2b (SEQ ID NO: 28), or SlMKS2c (SEQ ID NO: 26).

In some embodiments, recombinant expression vectors comprising various nucleic acids are provided. Further included are cells that comprise these recombinant expression vectors, where in some cases the recombinant expression vector can be integrated into the genomic DNA of the cell. Examples of cells include prokaryotes and eukaryotes, such as plant cells. Also included are multicellular organisms comprising the recombinant expression vector, wherein the recombinant expression vector is integrated into the genomic DNA of the organism.

Methods of making a methylketone or methylketone intermediate are provided. These methods comprise hydrolyzing a 3-ketoacyl intermediate with a recombinant Methylketone Synthase 2 (MKS2) to form a 3-ketoacid. In some embodiments, the 3-ketoacyl intermediate comprises 3-ketoacyl-ACP or 3-ketoacyl-CoA. The recombinant MKS2 may comprise SlMKS2 (SEQ ID NO: 3) or ShMKS2 (SEQ ID NO: 4) and may also comprise various polypeptides, including transit peptides, which can include ShMKS2 (SEQ ID NO: 25), SlMKS2a (SEQ ID NO: 27), SlMKS2b (SEQ ID NO: 28), or SlMKS2c (SEQ ID NO: 26). Such methods may further comprise decarboxylating the 3-ketoacid to form a 2-methylketone. For example, decarboxylating may comprise heating or treating with acid and heat. In some instances, the decarboxylating can include decarboxylating the 3-ketoacid with Methylketone Synthase 1 (MKS1) to form a 2-methylketone. The MKS1 may comprise ShMKS1 (SEQ ID NO: 17), and may comprises SlMKS1a (SEQ ID NO: 18), SlMKS1b (SEQ ID NO: 19), SlMKS1d (SEQ ID NO: 20), or SlMKS1e (SEQ ID NO: 21). In some embodiments, the hydrolyzing occurs within or proximate to a cell and the cell expresses the recombinant Methylketone Synthase 2 (MKS2).

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1. Distribution plot of 2-tridecanone (2TD) levels in the cultivated tomato S. lycopersicum (var. M82), the wild species S. habrochaites f glabratum (accession PI126449; PI), the F1 hybrid of these parents, the F2 segregating population derived from self-pollinated F1, progeny derived from the first and second backcrossing of the F1 with M82 (BC1-M82, BC2-M82) and first backcrossing with PI (BC1-PI). 2TD levels were log-transformed. The line across each diamond represents the group mean. The vertical span of each diamond represents the 95% confidence interval for each group.

FIG. 2. Variation and distribution of the gland shape and 2TD content in an interspecific F2 population originated from a cross between the cultivated and wild species. (A) A representative binocular image of a Type VI glandular trichome on the abaxial surface of a young leaflet from segregating progeny. Plants were categorized as having three types of trichomes based on six independent photos that were taken from different leaves: “M82-like shape” in which the top cells of the trichome are partially separated (left), “Intermediate shape” in which the cells are merged into a square-like shape (middle), and “PI shape” in which the cells are merged into a globular shape (right). (B) Distribution of the F2 plants among the three trichome categories. (C) Distribution of the 2TD content in the three trichome categories. Horizontal and vertical lines (black crosses) represent the average and 95% confidence limits, respectively.

FIG. 3. Mosaic plot of trichome shape in each of the genotypic classes of MKS1. C, cultivated (M82) allele; W, wild-species (PI126449) allele.

FIG. 4. (A) Association analysis of candidate genes and trichome characteristics with leaf 2TD content. The multiple regression model includes all of the tested factors for which a significant effect was found. The effect is defined as the contribution of one level/allele of the tested factor to the 2TD content. (B) Accumulated variation explained by the model (R2) with each additional factor. (C) Expression of MKS1, ACC and MaCoA-ACP trans in trichomes of the wild species S. habrochaites f glabratum (accession PI126449; PI) and the cultivated tomato S. lycopersicum (var. M82). mRNA abundance in isolated trichomes was determined by qRT-PCR. Expression levels for the various samples were normalized to the expression of Actin. Data are averages of three biological replicates, and error bars represent STE.

FIG. 5. Amino acid sequence alignment of SlMKS2, ShMKS2, and related proteins. White letters on black background indicate identical amino acids in the majority (>8) of sequences. White letters on gray background indicate conserved amino acid substitutions. The asterisk indicates the catalytic aspartate residue identified in the enzyme 4-hydroxybenzoyl-CoA thioesterase (4HBT). The complete cDNA sequences of SlMKS2 and ShMKS2 indicate that the open reading frames begin as indicated in this figure. At, Arabidopsis thaliana, Atr, Amborella trichopoda; Gh, Gossypium hirsutum; Gm, Glycine max; Hl, Humulus lupulus (L. cultivar Phoenix); Os, Oryza sativa; Pa, Prunus armeniaca; Pi, Petunia integrifolia subsp. Inflate; Pg, Picea glauca; Ps, Pseudomonas sp. (strain CBS-3); Sh, Solanum habrochaites; Sl, Solanum lycopersicum; Vv, Vitis vinifera. SsDHNACT—Synechosystis sp. PCC6803 1,4-dihydroxy-2-naphthoyl-CoA thioesterase. Accession numbers: Atr—FD440753, Gh—DT554179, Gm—AW394535, Hl—EX521228, Os—CAE01692, Pi—AAS90598, Pg—EX412733, Vv—CA042155, SsDHNACT—NP442358. The proteins shown in the figure correspond to the following Sequence Listing Identifiers: H1GD249868 (SEQ ID NO: 1); GmAW394535 (SEQ ID NO: 2); SlMKS2 (SEQ ID NO: 3); ShMKS2 (SEQ ID NO: 4); PiAAS90598 (SEQ ID NO: 5); PgEX412733 (SEQ ID NO: 6); VvCAO42155 (SEQ ID NO: 7); GhDT554179 (SEQ ID NO: 8); AtrFD440753 (SEQ ID NO: 9); AT1G68260.1 (SEQ ID NO: 10); AT1G68280.1 (SEQ ID NO: 11); AT1G35290.1 (SEQ ID NO: 12); AT1G35250.1 (SEQ ID NO: 13); OsCAE01692 (SEQ ID NO: 14); SsDHNACT (SEQ ID NO: 15); and Ps4HBT (SEQ ID NO: 16).

FIG. 6. Association analysis of MKS2 and other MK-modulating loci with 2TD levels. (A) Allelic distribution at the MKS2 locus in the F2 population using HRM marker: 1, homozygous for M82 allele; 2, heterozygous; 3, homozygous for PI allele. (B) Multiple regression analysis for testing the association of candidate genes and trichome characteristics to the 2TD content in the leaves. The model includes all the tested factors for which a significant effect was found. The effect is defined as the contribution of one level/allele of the tested factor to the 2TD content. (C) Accumulated variation explained by the model (R2) with each additional factor. (D) Expression of MKS2 in trichomes of the wild species S. habrochaites f glabratum (accession PI126449; PI) and the cultivated tomato S. lycopersicum (var. M82). mRNA abundance in isolated trichomes was determined by qRT-PCR. Expression levels were normalized to the expression of Actin. Data are averages of three biological replicates, and error bars represent STE.

FIG. 7. Genetic interaction of the MKS1 and MKS2 loci. (A) 2TD least square (LS) means plot. The X-axis represents the genotypes of the MKS1 locus: 1, homozygous for the M82 allele; 2, heterozygous or homozygous for the PI allele. The lines represent different genotypes of the MKS2 locus: 1, homozygous for the M82 allele; 2, heterozygous; 3, homozygous for the PI allele. (B) Levels of the haplotype factor. MKS1: 1, homozygous for M82 allele; 2, heterozygous or homozygous for the PI allele. MKS2: 1, homozygous for the cultivated allele; 2, heterozygous; 3, homozygous for the PI allele. (C) Accumulated variation explained by the model (R2) with each additional factor.

FIG. 8. GC-MS analysis of volatile compounds produced in E. coli when ShMKS2 (A) or SlMKS2 (B) are expressed. Peak labeled “1” is 2-tridecenone but the position of the double bond is not determined.

FIG. 9. Illustration of the hydrolysis (I) and decarboxylation (II) steps that mediate MK biosynthesis from 3-ketoacyl intermediates. R represents either Acyl Carrier Protein (ACP) or Coenzyme A (CoA).

FIG. 10. Markers developed for the candidate genes in the MK-pathway and genotyping of an interspecific F2 population. (A) Representative gel image of ACP1 PCR products separated on a 1% (w/v) agarose gel. Image includes products from the control DNA of the wild species (PI), cultivated line (M82), and portion of the F2 population. Upper single band (200 bp) represents homozygous for the wild allele. Lower single band (150 bp) represents homozygous for the cultivated allele. Triple band represents heterozygous (the third band could be a chimeric product of the two alleles). (B) Representative gel image of screening the F2 population with a CAPS marker for the KAS1 locus (PCR products digested with Taq1). Upper single band (1200 bp) represents homozygous for the wild allele, lower single band (1000 bp) represents homozygous for the cultivated allele (200 by band is not in the picture) and double band represents heterozygous. (C-H) Example of genotyping the F2 population with HRM markers at the following loci: (C) Acetyl-CoA carboxylase (ACC); (D) Malonyl-CoA:ACP transacylase (MaCoA-ACP trans); (E) 3-ketoacyl-ACP synthase III (KAS3); (F) 2,3-trans-enoyl-ACP reductase; (G) Acyl carrier protein 2 (ACPII); (H) Methylketone synthase 1 (MKS1). Arrows point to the three genotypes in each locus: (1) homozygous for the wild allele, (2) heterozygous, (3) homozygous for the cultivated allele.

FIG. 11. Genetic mapping using Solanum pennellii introgression lines (ILs) with the HRM technology. (A) Screening the ILs using an HRM marker for MKS1. Introgression line 1-4 is the only line that shares the same pattern with S. pennellii, thereby localizing it to bin 1-I (B). Chromosomal location of loci ACC and MaCoA-ACP trans is shown as well.

FIG. 12. Homology model of tomato MKS2 templated on the structure of a putative thioesterase from Thermus thermophilus (PDB ID: 1Z54). The hotdog-fold absolutely conserved homodimeric interface is represented both in the foreground (blue and gold monomers) and background (green and rose). The less conserved tetrameric assembly also depicted here is found in both 1Z54 and 4HBT (PDB ID: 1L09). External binding of phosphopantetheinylated cofactors at the edges of the conserved homodimeric interface delivers thioester-activated substrates to one of four identical internal active sites, illustrated here by two stick molecules of coenzyme A borrowed from another related 4HBT-subfamily crystal structure (PDB ID: 2CYE).

FIG. 13: A schematic reaction sequence for the synthesis of straight-chain methylketones. 3-Ketoacyl-ACP or 3-ketoacyl-CoA intermediates of fatty acid synthesis and degradation, respectively, are first hydrolyzed, and the resulting 3-ketoacids are then decarboxylated to give the corresponding 2-methylketone.

FIG. 14: Comparison of the protein sequence of S. habrochaites glabratum ShMKS1 with homologous (MKS1-Like, or MKS1L) sequences from S. lycopersicum, Vitis vinifera (grape), Populus trichocarpa (poplar), and Arabidopsis thaliana. Accession numbers are: ShMKS1-GU987105; SlMKS1a-GU987107; SlMKS1b-GU987108; SlMKS1d-GU987110; SlMKS1e-GU987111, PtMKS1L (MKS1-like)-XM_{—}002313048, VvMKS1L-XM_{—}002284871. AtMES3 is At2g23610. The proteins shown in the figure correspond to the following Sequence Listing Identifiers: ShMKS1 (SEQ ID NO: 17); SlMKS1a (SEQ ID NO: 18); SlMKS1b (SEQ ID NO: 19); SlMKS1d (SEQ ID NO: 20); SlMKS1e (SEQ ID NO: 21); PtMKS1L (SEQ ID NO: 22); VvMKS1L (SEQ ID NO: 23); and AtMES3 (SEQ ID NO: 24).

FIG. 15: Comparison of the protein sequence of S. habrochaites glabratum ShMKS2 with homologous sequences from S. lycopersicum, Arabidopsis thaliana, and Pseudomonas sp. Accession numbers are: ShMKS2-GU987106; SlMKS2a-GU987112; SlMKS2b-GU9877113; SlMKS2c-GU987114; Ps4HB-EF569604. The initiating MET codon used to produce ShMKS2 protein without the transit peptide is underlined. The proteins shown in the figure correspond to the following Sequence Listing Identifiers: ShMKS2 with transit peptide (SEQ ID NO: 25); SlMKS2c with transit peptide (SEQ ID NO: 26); SlMKS2a with transit peptide (SEQ ID NO: 27); SlMKS2b with transit peptide (SEQ ID NO: 28); AT1G68260 (SEQ ID NO: 10); AT1G68280 (SEQ ID NO: 11); AT1G35290 (SEQ ID NO: 12); AT1G35250 (SEQ ID NO: 13); and Ps4HBT (SEQ ID NO: 16).

FIG. 16: Subcellular localization of ShMKS2-eGFP fusion proteins in Nicotiana benthamiana leaf cells. The panels shown on the left exhibit green fluorescence from eGFP, the panels in the middle show red fluorescence from plastidic chlorophyll, and each panel in the right column exhibits an overlay of the two panels to its left. (A-C) Tobacco cells infiltrated with an empty binary vector; (D-F) tobacco cells infiltrated with a binary vector carrying the complete opening reading frame of ShMKS2 fused to eGFP; and (G-I), tobacco cells infiltrated with a binary vector carrying the ShMKS2 gene lacking the putative transit peptide and fused to eGFP. Bar=10 μm.

FIG. 17: Total amount of methylketones found in spent media of E. coli cells expressing ShMKS1, ShMKS2, and ShMKS2 (D79A) (all missing the transit peptide-coding region) from the pEXP-TOPO-CT bacterial expression vector. Cells were grown and spent media were collected and treated as described in the Examples, Materials, and Methods. Control 1 cells expressed Clarkia breweri isoeugenol synthase1 (CbIGS1) on pEXP-TOPO-CT, as described by Koeduka et al., (2008) The multiple phenylpropene synthases in both Clarkia breweri and Petunia hybrida represent two distinct protein lineages, Plant J 54: 362-374. Control 2 cells contained a pEXP-TOPO-CT vector with no insert. Values are averages±SE calculated from three experiments.

FIG. 18: Methylketone production by E. coli cell expressing ShMKS2. Treated and non-treated spent media of E. coli cells expressing ShMKS2 (without the transit peptide-coding region) were extracted with hexane and the methylketone content was measured by GC-MS. Treatments included heat, acid and heat, purified ShMKS1 protein in phosphate buffer and phosphate buffer alone. Values are averages±SE calculated from three experiments.

FIG. 19: Decarboxylase activity assays for ShMKS1 and ShMKS2 using 3-ketomyristic acid as the substrate. Purified recombinant proteins were assayed as described in the Examples, Materials, and Methods, and the mean values and SD values were calculated from three replicates and given as nM of 2-tridecanone formed per microgram protein per minute.

FIG. 20: Thioesterase activity assays for ShMKS1 and ShMKS2. To a 500 μL solution of enzymatically prepared 3-ketomyristoyl-ACP (see Examples, Materials, and Methods), 20 μL solution of the following were added: 1) enzyme buffer, 2) 2.5 μg ShMKS1 in buffer 3), enzyme buffer, 4) 2.5 μg ShMKS2, and 5) 2.5 μg ShMKS1 and 2.5 μg ShMKS2. Each reaction was incubated for 30 min at 23° C., after which the reaction solution was either extracted directly with hexane (samples 1, 2 and 5) or first treated with acid and heated at 75° C. for 30 min (reactions 3 and 4), then cooled down to room temperature and extracted with hexane. Hexane extracts were analyzed by GCMS. Mean values and SD values were calculated from three replicates.

FIG. 21: Nucleotide sequence (SEQ ID NO: 29) of SlMKS1a and the amino acid sequence (SEQ ID NO: 18) of the protein it encodes. Shown here are 494 nucleotides upstream of the initiating ATG codon, three exons, two introns and 500 nucleotides downstream of the TAA stop codon. Exon sequences are in capital letters, with the amino acids sequence above the codons. The sequence is derived from Scaffold 05477 (available online at solgenomics.net/tools/blast/show_match_seq.pl?blast_db_id=95&id=scaffold05477).

FIGS. 22 A & B: Nucleotide sequence (SEQ ID NO: 30) of SlMKS1b and the amino acid sequence (SEQ ID NO: 19) of the protein it encodes. Shown here are 1000 nucleotides upstream of the initiating ATG codon, three exons, two introns and 500 nucleotides downstream of the TAA stop codon. Exon sequences are in capital letters, with the amino acids sequence above the codons. The sequence is derived from Scaffold 05477.

FIGS. 23 A & B: Nucleotide sequence (SEQ ID NO: 31) of SlMKS1d and the amino acid sequence (SEQ ID NO: 20) of the protein it encodes. Shown here are 1000 nucleotides upstream of the initiating ATG codon, three exons, two introns and 500 nucleotides downstream of the TGA stop codon. Exon sequences are in red capital letters, with the amino acids sequence above the codons. The sequence is derived from Scaffold 05390 (available online at solgenomics.net/tools/blast/show_match_seq.pl?blast_db_id=95&id=scaffold05390). The upstream sequence of this gene, unlike those of the other SlMKS1 genes, contains an ATG triplet in-frame that could possible serve as an initialing codon to make a larger N-terminal extension. However, 5′RACE experiments indicated that the 5′ end of the transcript occurs downstream to the ATG triplet. The yellow highlighted nucleotide shows the position of the first nucleotide in the longest 5′RACE product. The arrow indicates the oligonucleotide primer used in the 5′RACE experiment.

FIGS. 24 A & B: Nucleotide sequence (SEQ ID NO: 32) of SlMKS1e and the amino acid sequence (SEQ ID NO: 21) of the protein it encodes. Shown here are 1000 nucleotides upstream of the initiating ATG codon, three exons, two introns and 500 nucleotides downstream of the TGA stop codon. Exon sequences are in capital letters, with the amino acids sequence above the codons. The sequence is derived from Scaffold 05390.

FIGS. 25 A & B: Nucleotide sequence (SEQ ID NO: 33) of SlMKS1c and the amino acid sequence (SEQ ID NO: 34) of the protein it encodes. Shown here are 1000 nucleotides upstream of the initiating ATG codon, three exons, two introns and 500 nucleotides downstream of the TAA stop codon. Exon sequences are in capital letters, with the amino acids sequence above the codons. The sequence is derived from Scaffolds 05477 and 00200 (00200 is an updated version of 05477) (available online at solgenomics.net/tools/blast/show_match_seq.pl?blast_db_id=95&id=scaffold00200). The premature stop codon is shaded.

FIG. 26: Nucleotide sequence (SEQ ID NO: 35) of ShMKS1 and the amino acid sequence (SEQ ID NO: 17) of the protein it encodes. Shown here are 1000 nucleotides upstream of the initiating ATG codon, three exons and two introns. Exon sequences are in capital letters, with the amino acids sequence above the codons. Oligonucleotide primers used for genomic PCR are underlined by arrow. Arrows 1 and 2 show the oligonucleotide primers used to isolate, by PCR, the genomic fragment carrying the gene. The promoter sequence was isolated by chromosomal walking (see the Examples, Materials, and Methods).

FIGS. 27 A, B, & C: Nucleotide sequence (SEQ ID NO: 36) of SlMKS2a and the amino acid sequence (SEQ ID NO: 27) of the protein it encodes. Shown here are 1000 nucleotides upstream of the initiating ATG codon, five exons, four introns and 500 nucleotides downstream of the TAA stop codon. Exon sequences are in capital letters, with the amino acids sequence above the codons. The sequence is derived from Scaffold 04161. Underlined codon is the first ATG codon in the previously characterized MKS2 cDNA. (available online at solgenomics.net/tools/blast/show_match_seq.pl?blast_db_id=95&id=scaffold04161).

FIGS. 28 A & B: Nucleotide sequence (SEQ ID NO: 37) of SlMKS2b and the amino acid sequence (SEQ ID NO: 28) of the protein it encodes. Shown here are nucleotides upstream of the initiating ATG codon, five exons, four introns and 500 nucleotides downstream of the TAA stop codon. Exon sequences are in capital letters, with the amino acids sequence above the codons. The sequence is derived from Scaffold 04161.

FIGS. 29 A & B: Nucleotide sequence (SEQ ID NO: 38) of SlMKS2c and the amino acid sequence (SEQ ID NO: 26) of the protein it encodes. Shown here are 1670 nucleotides upstream of the initiating ATG codon, five exons, four introns and 500 nucleotides downstream of the TAG stop codon. Exon sequences are in capital letters, with the amino acids sequence above the codons. The sequence is derived from Scaffold 04161. Underlined codon is in the equivalent position of the first ATG codon in the previously characterized ShMKS2 cDNA. The arrow indicates the oligonucleotide used for isolating the promoter of ShMKS2.

FIG. 30: Nucleotide sequence (SEQ ID NO: 39) of ShMKS2 and the amino acid sequence (SEQ ID NO: 25) of the protein it encodes. Shown here are 1459 nucleotides upstream of the initiating ATG codon, five exons and four introns. Exon sequences are in capital letters, with the amino acids sequence above the codons. Underlined codon is the first ATG codon in the previously characterized ShMKS2 cDNA. Arrows indicate oligonucleotide primers used in genomic PCR and 5′RACE experiments. Arrow 2 was the reverse primer for isolating the promoter; arrows 1 and 4 were used for genomic PCR; arrows 3 and 4 were used for 5′RACE experiments. The first nucleotide of the transcript, identified in the 5′RACE experiments, is shaded.

FIG. 31: GC-MS analysis of methylketones produced by E. coli expressing the Arabidopsis gene At1g68260.

FIGS. 32 A & B: Gas chromatography of reaction products produced by enzymes from MKS2 genes expressed in E. coli.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

Genetic analysis of interspecific populations derived from crosses between the wild tomato species Solanum habrochaites f glabratum, which synthesizes and accumulates insecticidal methylketones (MK), mostly 2-undecanone and 2-tridecanone, in glandular trichomes, and Solanum lycopersicum (cultivated tomato) which does not, demonstrated that several genetic loci contribute to MK metabolism in the wild species. A strong correlation was found between the shape of the glandular trichomes and their MK content, and significant associations were seen between allelic states of three genes and the amount of MK produced by the plant. Two genes belong to the fatty acid biosynthetic pathway and the third is Methylketone Synthase 1 (MKS1) that mediates divergence to MK from β-ketoacyl intermediates. Comparative transcriptome analysis of the glandular trichomes of F2 progeny grouped into low- and high-MK-containing plants identified several additional genes whose transcripts were either more or less abundant in the high-MK bulk. In particular, a wild-species specific transcript for a gene which we named Methylketone Synthase 2 (MKS2), encoding a protein with some similarity to a well-characterized bacterial thioesterase, was approximately 300-fold more highly expressed in F2 plants with high MK content than in those with low MK content. Genetic analysis in the segregating population showed that MKS2's significant contribution to MK accumulation is mediated by an epistatic relationship with MKS1. Furthermore, heterologous expression of MKS2 in Escherichia coli resulted in the production of methylketones in this host.

The trichomes of the wild tomato species Solanum habrochaites subspecies glabratum synthesize and store high levels of methylketones, primarily 2-tridecanone and 2-undecanone, which protect the plant against various herbivorous insects. We identified cDNAs encoding two proteins necessary for methylketone biosynthesis, designated methylketone synthase 1 (ShMKS1) and ShMKS2. We further report isolation of genomic sequences encoding ShMKS1 and ShMKS2 as well as the homologous genes from the cultivated tomato, S. lycopersicum. We show that a full-length transcript of ShMKS2 encodes a protein that is localized in the plastids. By expressing ShMKS1 and ShMKS2 in E. coli and analyzing the products formed, as well as by performing in vitro assays with both ShMKS1 and ShMKS2, we have determined that ShMKS2 acts as a thioesterase that hydrolyzes 3-ketoacyl-ACPs (plastid localized intermediates of fatty acid biosynthesis) to release 3-ketoacids, and that ShMKS1 subsequently catalyzes the decarboxylation of these liberated 3-ketoacids, forming the methylketone products. Genes encoding proteins with high similarity to ShMKS2, a member of the “hot-dog fold” protein family that is known to include other thioesterases in non-plant organisms, are present in plant species outside the genus Solanum. We show that a related enzyme from Arabidopsis thaliana also produces 3-ketoacids when recombinantly expressed in E. coli. Thus, the thioesterase activity of proteins in this family appears to be ancient. In contrast, the 3-ketoacid decarboxylase activity of ShMKS1, which belongs to the α/β-hydrolase fold superfamily, appears to have emerged more recently, possibly within the genus Solanum.

Plants exhibit a large range of chemical and morphological variation, reflecting different adaptations to mediating their interactions with the biotic and abiotic environment throughout their life cycle. Some plant chemicals are lipophilic (oily) compounds that have high vapor pressure and therefore volatilize easily when exposed to air. Such volatiles can serve as signal molecules that either attract or repel animals. Many such compounds are also toxic and can damage a predatory organism through external or internal contact, and are therefore synthesized in dedicated cells that also serve to store them. In particular, such compounds may be synthesized and accumulated in small epidermal cell extensions on the surface of leaves, stems, and reproductive tissues called glandular trichomes. Since the initial work on glandular trichomes in mint, various studies involving transcriptomics, proteomics, and metabolomics have indicated that entire metabolic pathways responsible for the production of such compounds operate within the trichomes and that these unique cells require the import of only the basic building blocks to make these chemicals.

The cultivated tomato Solanum lycopersicum and its wild relative Solanum habrochaites represent two of the twelve main taxa found within the Solanum section Lycopersicon. While only limited genetic diversity is found among the cultivated S. lycopersicum accessions, a wide range of variance is found in the wild relatives. This richness of genetic polymorphism is well reflected by the wide repertoire and quantity of specialized compounds accumulated in their trichomes, including mono and sesquiterpenes, acyl sugars, and methylketones (MK). Up to seven types of trichomes have been reported in the various Solanum species. One of these trichome types that has been investigated in some detail is the Type VI glandular trichome, which is composed of a stalk cell with four cells at the top that form a mushroom-like shape; a cuticular sac wrapped around these cells allows accumulation of secreted compounds similar to an inflating balloon. We have shown that in the wild species Solanum habrochaites f glabratum, the Type VI glandular trichomes, which are present at high density on both the leaf surfaces and stems, contain two main MK compounds, 2-tridecanone (2TD, containing a 13-C backbone) and 2-undecanone (2UD, containing an 11-C backbone), as well as some 2-pentadecanone (containing a 15-C backbone) and a few other unidentified MK compounds. These MKs are synthesized and accumulate to high levels in these trichomes, up to 5500 μg/g leaf fresh weight.

Analysis of a Type VI-specific EST database from a MK-producing S. habrochaites f glabratum (accession PI126449) showed that transcripts of genes encoding plastidic enzymes of fatty acid biosynthesis are highly represented, in contrast to their relatively low representation in a line that does not make MK (accession LA1777). The comparative analysis of the two ETS databases also led to the isolation and characterization of a novel gene encoding a protein belonging to the α/β hydrolase family, which was specifically and exclusively expressed in Type VI trichomes of methylketone-producing plants but not in non-producers. Although the protein did not appear to have a transit peptide, the results of plastid import experiments indicated that it could be imported into the plastids.

Since 3-ketoacids are inherently unstable and undergo spontaneous decarboxylation, albeit at low rate at ambient temperature, the evidence of elevated levels of fatty acid biosynthesis in these trichomes suggested that the observed straight-chain methylketones such as 2TD and 2UD could be derived from enzymatic or non-enzymatic decarboxylation of the respective Cₙ₊₁ 3-ketoacids. In plants, 3-ketoacyls of fatty acids mostly occur in plastids (as 3-ketoacyl-ACPs) as intermediates in the fatty acid biosynthesis pathway, and in peroxisomes (as 3-ketoacyl-CoA) as intermediates in the fatty acid degradation pathway (available online at lipids.plantbiology.msu.edu/?q=lipids/genesurvey/). The identification of a plastid-localized hydrolase led us to carry out in vitro assays with this enzyme, subsequently designated as Methylketone Synthase 1 (MKS1), with the C12, C14, and C16 3-ketoacyl-ACPs as substrates. In these assays, the respective C11, C13 and C15 MKs were produced, indicating that MKS1 is capable of both hydrolyzing the thioester bond and decarboxylating the resulting 3-ketoacid intermediate. However, it was noted that the turnover rate of the enzyme was unusually low.

Crosses between MK-producing and non-producing lines followed by segregation analysis have indicated that the ability to produce MK requires multiple quantitative trait loci in addition to MKS1. Consequently, it has not been possible to breed cultivated tomato lines that produce high levels of MK in their glands. It is likely that the trait of MK production in S. habrochaites evolved through multiple morphological and biochemical changes that took place gradually during evolution.

To uncover the additional factors influencing MK production, we took a quantitative genetic approach to identify QTLs that might affect MK production, including genes encoding biosynthetic enzymes, and tested the possible relationship between trichome characteristics and chemical content. In addition, comparative transcriptomic analysis was used to identify new genes whose differential expression is correlated with MK production in interspecific populations.

Morphological and Chemical Analyses of Interspecific Populations Derived from Crosses Between the Cultivated Tomato and S. habrochaites f glabratum

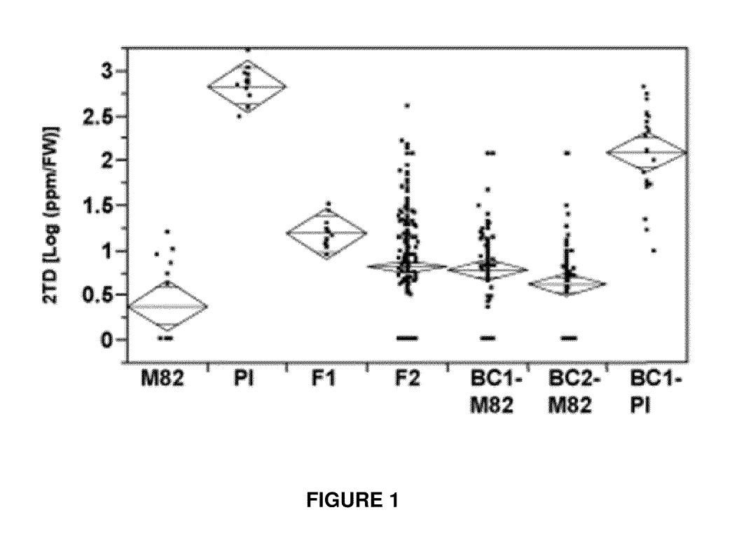

The chemical profiles of leaves of the cultivated tomato S. lycopersicum (var. M82) and the wild species S. habrochaites f glabratum (accession PI126449) differ in their shape and chemical content. In particular, leaves of the cultivated tomato contain little or no MK while leaves of the wild species contain high levels of 2UD and 2TD which are synthesized and stored in the Type VI glandular trichomes on the leaf surface. A series of crosses were conducted between these accessions to genetically dissect the contribution of candidate genes to MK content. Tomato plants of different genetic backgrounds were then evaluated, including the two parental lines: Solanum habrochaites f glabratum (accession PI126449; PI) and Solanum lycopersicum var. M82 (14 plants of each), F1 hybrids of these parents (14 plants), an F2 segregating population derived from self-pollinated F1 (245 plants), progeny derived from the first and second backcrossing of F1 with M82 (82 and 72 plants, respectively), and progeny derived from the first backcrossing of F1 with PI (22 plants). All plants were randomly planted and from each, six young leaflets were removed for chemical characterization and 2TD level determination, since 2TD is the major MK produced in the parental wild-species. Overall, the 2TD levels of most F2 progeny were more similar to the cultivated tomato parent (FIG. 1). This, combined with the observation of very low values in the backcrossed (BC) generations indicated polygenic inheritance of this trait and suggested the recessive characteristic of the wild-species alleles that participate in this pathway.

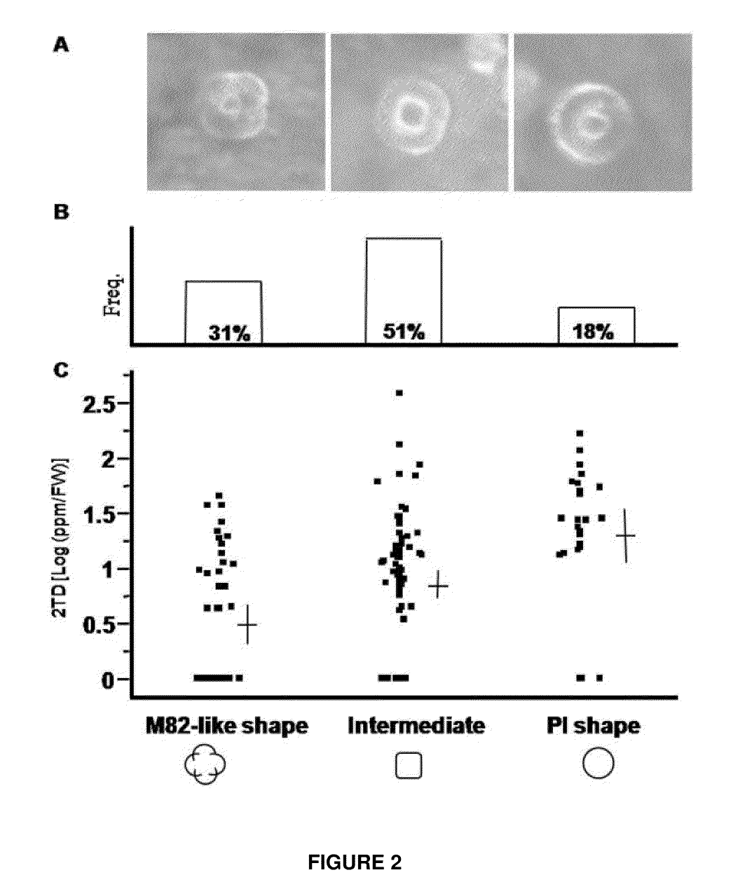

Digital images of leaflet surfaces were taken to determine trichome density and its association with MK accumulation. While analyzing these images, we noticed that the F2 population segregates not only for trichome number, but also for trichome shape. This observation is in agreement with previously described distinctions in trichome shape between cultivated and wild species of tomato. While none of the F2 plants showed clear separation of the cells at the tip of the trichomes (as the trichomes of M82), 31% of the population had Type VI trichomes with partial separation of these cells (M82-like; FIG. 2A,B), 18% of the F2 progeny had round Type VI trichomes, basically identical in shape to those of the wild species (PI shape; FIG. 2A, B), and in 51% of the plants, the cells of the Type VI trichomes were not separated similar to the M82 parent, but the trichome appeared more square than round. The latter morphology was designated as intermediate (FIG. 2A, B). Interestingly, on average, plants with PI-shaped trichomes accumulated the highest levels of MK, plants with the intermediate trichomes accumulated intermediate levels of MK and plants with M82-like trichomes accumulated the lowest levels of MK (FIG. 2C). The mean MK values of these three groups differed significantly from each other (Tukey HSD; α=0.005).

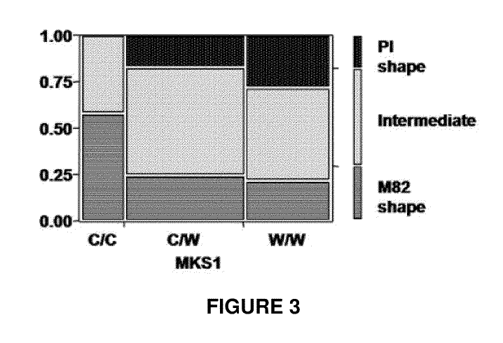

Since MKS1 is a major gene in the MK pathway, we looked for possible relationship between the MKS1 genotype and the shape of the trichomes in the interspecific segregating F2 population. There were significant differences (Pearson test, P<0.003) in the frequencies of the three groups of plants with different Type VI trichome shapes among the three genotypes of MKS1. In particular, no F2 progeny exhibited PI-shaped trichomes among homozygotes for the cultivated allele of MKS1 (C/C), and the trichomes of most of the plants in this group bore an M82-like shape (FIG. 3).

Association Between Candidate Genes, Trichome Characteristics, and MK Content

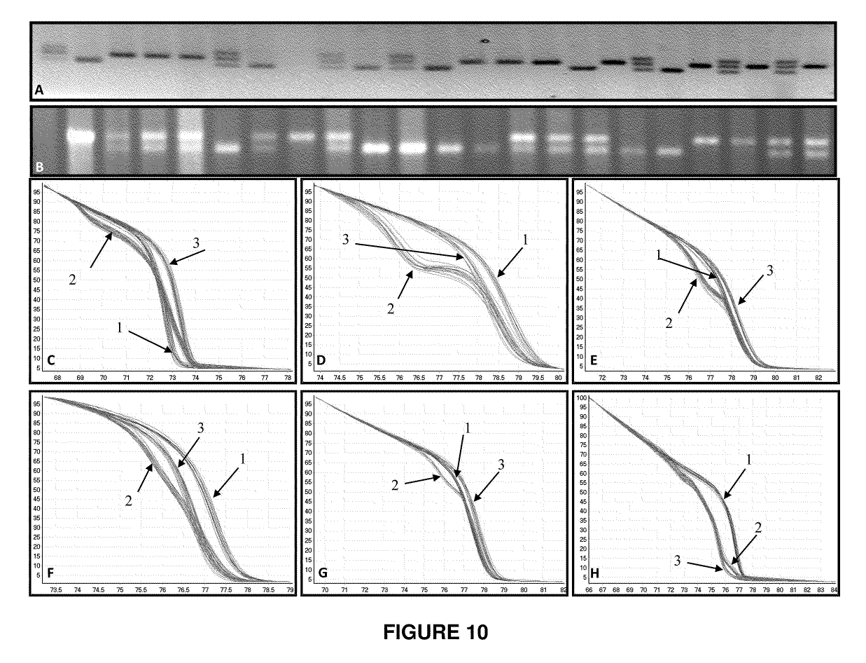

The association between variation in candidate structural genes and 2TD content was examined in genetic mapping experiments employing these genes as simple PCR markers, cleaved amplified polymorphism sequences (CAPS), or single-nucleotide polymorphism (SNP) markers, after aligning the open reading frame (ORF) of the alleles from both species and designing amplicons flanking the indel, SNP or restriction site (see Examples, Materials, and Methods section). The latter approach used high-resolution melt technology (HRM Assay Design and Analysis, C or Protocol 6000, 2006; FIG. 10).

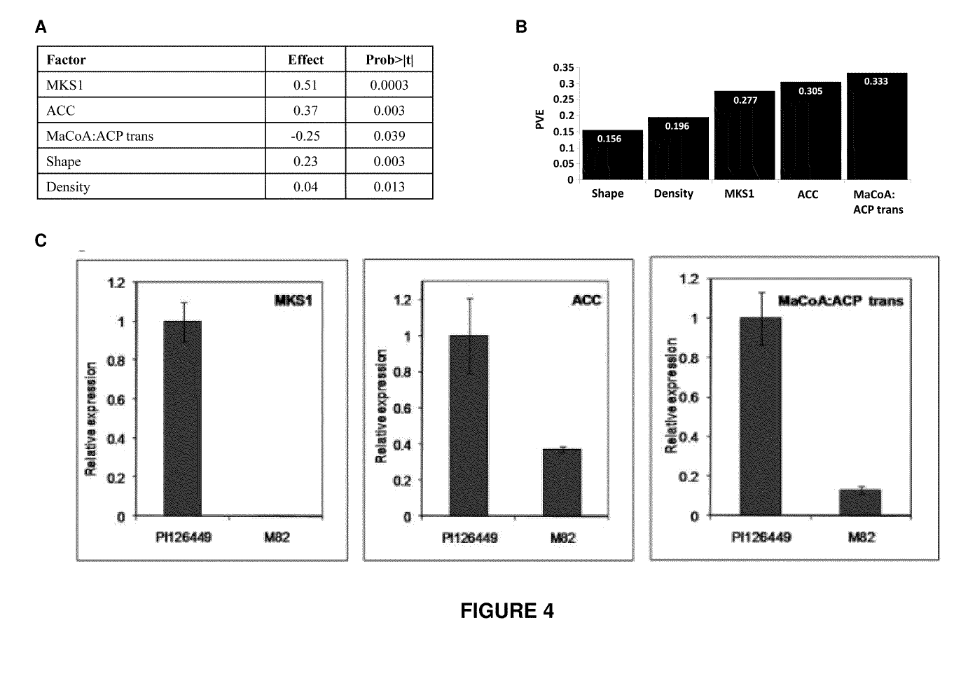

Since MK are derived from fatty acids, we examined the following genes from the fatty acid biosynthetic pathway (available online at lipids.plantbiology.msu.edu/?q=lipids/genesurvey/) as genetic markers: acetyl-CoA carboxylase (ACC), malonyl-CoA:ACP transacylase (MaCoA-ACP trans), 3-ketoacyl-ACP synthase III (KASIII), 2,3-trans-enoyl-ACP reductase, 3-ketoacyl-ACP synthase I (KASI) and Acyl carrier proteins 1 and 2 (ACP1 and ACP2). The MKS1 locus was also included in the genetic screening. Association test was conducted by multiple regression analysis using the allelic state of the different genes in each F2 progeny and trichomes characters as a predictor of 2TD level. This analysis (power=0.999) showed that segregating progeny carrying the wild allele in two loci (MKS1 and ACC) contain significantly (P<0.0003 and P<0.003, respectively) higher amounts of 2TD (FIG. 4A), while for the MaCoA-ACP trans locus, the opposite trend was observed, i.e. plants carrying the wild-species allele had, on average, significantly (P<0.039) less 2TD content. In addition, a significant positive correlation between the density and shape of the trichomes and the amount of MK in the leaves was found (FIG. 4A). This multiple regression reinforced the previous results indicating an association between trichome morphology and 2TD levels (FIG. 2), and overall this model explained approximately one-third of the total 2TD phenotypic variation in the F2 population (R2=0.333; FIG. 4B). To test whether the three significantly candidate genes associated with 2TD levels in the segregating population (MKS1, ACC and MaCoA-ACP trans), exhibit differential expression between the wild and cultivated species, quantitative reverse-transcription PCR (qRT-PCR) approach was taken. Primer pairs that fully matched both alleles were designed for each gene and qRT-PCR was conducted using RNA from trichomes of both accessions (see Examples, Materials and Methods). MKS1, ACC and MaCoA-ACP trans showed 355, 2.7 and 7.7-fold higher expression, respectively, in the trichomes of the PI parent vs. those of M82 parent (FIG. 4C).

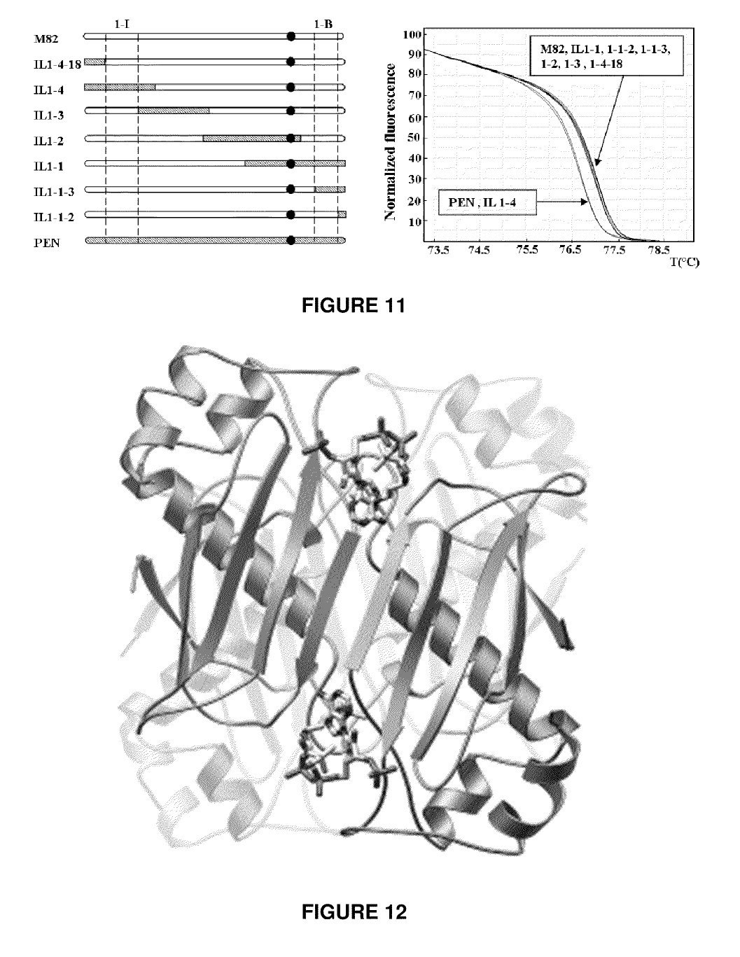

Mapping these three genes on the tomato genome using the Solanum pennellii introgression lines population identified genes ACC and MaCoA-ACP trans on chromosome 1, bin 1-B. Based on the F2 mapping population, the two loci are 8.8 cM apart (39 recombination events in 220 F2 progeny successfully scored for both loci). MKS1 was localized to bin 1-I, more than 50 cM away (FIG. 11). There were no significant linkages with any of the other loci tested.

Transcriptome Analysis of Trichomes from Bulked Segregants

Bulked segregant analysis was used to compare transcriptomes of plants with low and high MK contents. Five plants with high levels of 2TD and five plants with no detectable 2TD were selected from the segregating F2 population (total of 245 plants) and propagated for this analysis. RNA from the trichomes of each of these two groups of plants (with high and low MK content) was extracted, reverse-transcribed, labeled and hybridized to a custom-made microarray containing tomato genes (see Examples, Materials and Methods). A comparison of the hybridization results revealed a number of genes whose transcripts were present at either higher or lower levels in the high-MK-containing plants relative to their low-MK counterparts (Table 1). In particular, one wild-species specific transcript of a gene which we subsequently designated Methylketone synthase 2 (MKS2; see below) was 337-fold more highly expressed in F2 plants with high vs. low MK content while a similar transcript, derived from the cultivated species, was 7.5-fold more highly expressed in the F2 plants with low vs. high MK content (Table 1).

TABLE 1
Microarray Analysis of Genes Differentially
Expressed in High-and Low-MK Bulk^{a}

| Gene code^{b} | Annotation | Ratio^{c} |
|---|---|---|
| DN167657 | A protein related to a Pseudomonas | +336.6 |
| | thioesterase (Sh allele) | |
| A1779239 | rRNA-16S ribosomal RNA | +62.6 |
| AF230371 | Allene oxide synthase | +46.7 |
| B1925004 | Plasma membrane intrinsic protein | +39 |
| B1931228 | Unknown | +27.3 |
| AW616884 | Dehydrodolichyl diphosphate synthase | +24.1 |
| DN169296 | DNA repair protein RAD23 | +18.3 |
| DB719610 | Calcium-binding EF hand family protein | +15.5 |
| DN168712 | RuBisCO small subunit 1A | +14.7 |
| DN169129 | Major latex protein-related (Sh allele) | +12.9 |
| AW039905 | Peroxisomal protein involved in | +11.8 |
| | the activation of fatty acids | |
| A1777019 | Unknown | +11.2 |
| AW615872 | Glycosyltransferase family 14 protein | +11.1 |
| BF097749 | Mitochondrial 26S ribosomal RNA protein | +9.2 |
| BW688217 | Unknown | +9.1 |
| BM412813 | Methyltransferase family 2 protein | +8.8 |
| DN170232 | Protein kinase | +8.4 |
| DN171038 | Casein kinase 1 protein family | +7.2 |
| DB683900 | X-Pro dipeptidase | −7.1 |
| BG131749 | A protein related to a Pseudomonas | −7.5 |
| | thioesterase (Sl allele) | |
| AI772024 | Unknown | −7.6 |
| DB722221 | Unknown | −7.7 |
| DN168641 | Photosystem II oxygen-evolving complex 23 | −7.8 |
| ES893822 | Cell wall protein precursor | −7.9 |
| BG643000 | Phospholipase A2 beta | −8.0 |
| BW690350 | GRAM domain-containing protein/ | −8.3 |
| | ABA-responsive protein-related | |
| AW034502 | Cytochrome P450, putative | −8.9 |
| BG1237766 | Aldo/keto reductase family protein | −9.5 |
| BI928231 | NAD-dependent epimerase/ | −10.1 |
| | dehydratase family protein | |
| BI932160 | UDP-glucoronosyl/UDP-glucosyl | −10.6 |
| | transferase family protein | |
| BG128416 | Unknown | −10.9 |
| AW624755 | Major latex protein related | −11.9 |
| ES896328 | Chaperonin | −12.5 |
| BE434841 | 3-Ketoacyl-ACP synthetase 2 nuclear gene | −15.2 |

^{a}cDNA sequences from the customized microarray that were upregulated or downregulated by more than sevenfold are shown.
^{b}Corresponding GenBank accession numbers with the highest similarity are shown.
^{c}Ratio depicts the difference in average ratios of high-MK over low-MK bulks in four hybridizations for all the probes that represent the same sequence.

All genes listed in the table showed significant difference (p<0.05) in hybridization intensity between high- and low-MK bulks.

MKS2 Shares Sequence Identity with Hotdog-Fold Thioesterases

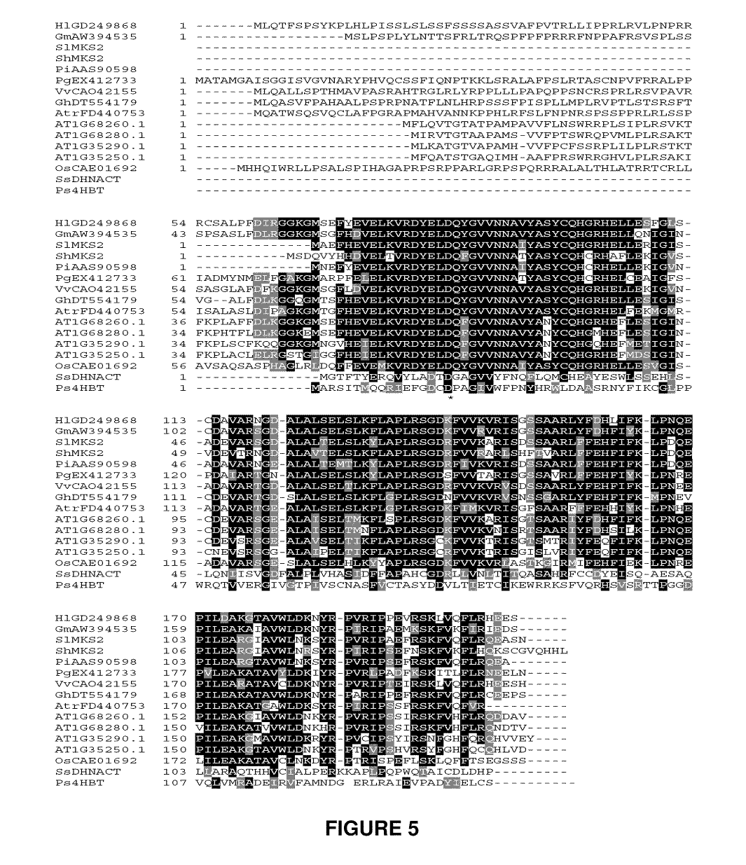

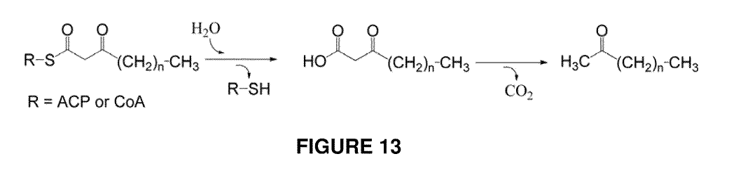

The MKS2 protein is 52% to 70% identical to several plant proteins with no proven functions, encoded by genes in the Arabidopsis and rice genomes and by many ESTs from various plant species of the angiosperm family, as well as from white spruce (Picea gluca) (FIG. 5). Sequence similarity established that the 149-residue protein encoded by MKS2 is a member of the 4-hydroxybenzoyl-coenzyme A thioesterase (4HBT) subfamily of hotdog-fold enzymes. Although only recently discovered, hotdog domains comprise a broad superfamily that is evolutionarily unrelated to the vast α/β-hydrolase-fold superfamily of (thio)esterases, of which MKS1 is a member. Notably, several hotdog-fold subfamilies required for typical fatty acid metabolism, including the FabA/FabZ 3-hydroxy-acyl-ACP dehydratases and the FatA/FatB saturated acyl-ACP thioesterases, occur instead as longer sequences that represent a tandem duplication of the hotdog fold. BLAST search revealed the MKS2 amino acid sequence to also be 34% identical (and 48% similar) over 131 aligned residues to a structurally characterized putative thioesterase from Thermus thermophilus (PDB code: 1Z54), which in turn shares 26% sequence identity and 51% similarity with 4HBT over 91 aligned residues. The crystallized 1Z54 protein backbone nearly perfectly overlays with known 4HBT structure (PDB code:1L09; FIG. 13). This ‘bridging’ 1Z54 sequence and crystal structure firmly establish the homology of MKS2 to the well-characterized 4HBT, and 1Z54 facilitates a structural homology-based examination of the MKS2 sequence. In addition, MKS2 conserves the catalytic Asp 17 of 4HBT, although our model predicts extensive substitution of juxtaposed residues in the MKS2 active-site cavity relative to any characterized 4HBT-subfamily member.

MKS2 is Associated with MK Content and Reveals an Epistatic Interaction with MKS1

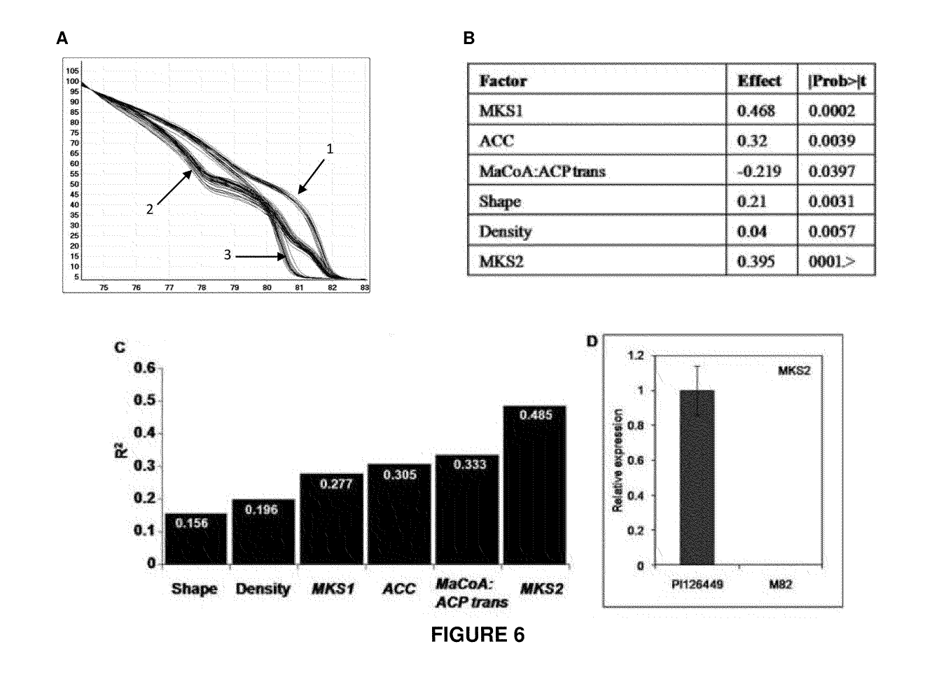

Nucleotide differences were used to employ the MKS2 gene as a DNA HRM marker (FIG. 6A) and to investigate the association between the allelic state in this locus and the 2TD-content variation in the segregating population. The allelic variation in MKS2 was significantly associated with 2TD content (P<0.0001), and ranked as the second-most contributing factor (after MKS1) among the loci thus far identified in this quantitative analysis (FIG. 6B). Moreover, inclusion of this locus in the multiple regression analysis increased the R² of the model from 0.333 to 0.485 (FIG. 6C). Expression analysis by qRT-PCR showed that MKS2 is 980-fold more highly expressed in the trichomes of the high-MK accumulator PI parent than in those of the M82 parent (FIG. 6D), similarly to MKS1, ACC and MaCoA-ACP trans (FIG. 4C).

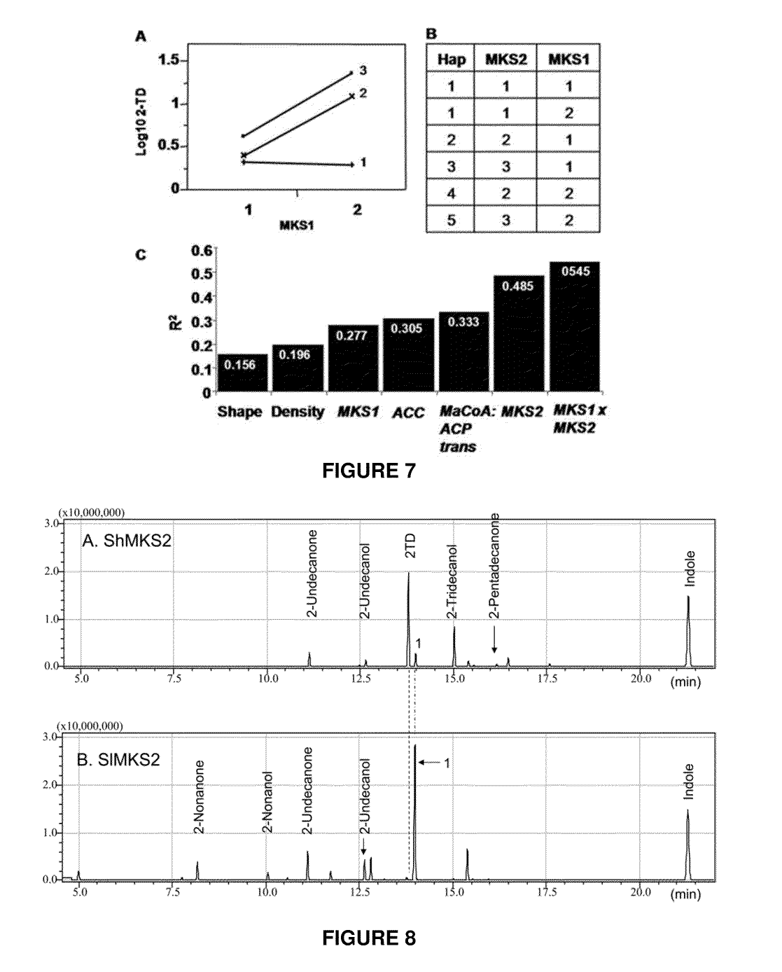

In an attempt to define possible epistatic interactions between the different genetic components of the MK network, the genetic factors that significantly contribute to MK variation in the test population were evaluated for possible two-way interactions. This analysis identified a single significant interaction between the MKS2 and MKS1 loci (FIG. 7A). The data showed that to achieve high levels of these compounds, the plant has to carry at least one wild-species allele in each of these two interacting loci. While MKS1 shows a dominant mode of inheritance, that of MKS2 is only partially so: all three genotypic classes differ significantly (d=0.5). Indeed, incorporation of the interaction between the two loci into new regression model by grouping the plants according to the two-locus haplotypes (FIG. 7B) increased the R² of the model from 0.485 to 0.545 (FIG. 7C).

Heterologous Expression of MKS2 in E. coli

To investigate the biochemical activity of MKS2, the full ORFs of the wild-species allele (ShMKS2) and the cultivated allele (SlMKS2) were amplified and ligated into an E. coli expression vector (see Examples, Materials and Methods). These vectors were introduced into E. coli BL21cells and ShMKS2 or SlMKS2 expression was induced by the addition of IPTG (see Examples, Materials and Methods). After induction and overnight growth, the culture was analyzed by solid-phase microextraction (SPME) of its headspace followed by gas chromatography-mass spectrometry (GC-MS; see Examples, Materials and Methods). The major compound in the headspace of the E. coli cells expressing ShMKS2 was identified as 2TD (FIG. 8A). Lower amounts of 2UD and 2-pentadecanone were also detected, as well as the reduced alcohol forms of 2UD and 2TD (i.e., 2-undecanol and 2-tridecanol). The headspace of the E. coli cells expressing SlMKS2 contained 2UD as well as 2-nonanone as the two main MK, and only trace amounts of 2TD. The headspace also contained 2-nonanol and 2-undecanol (FIG. 8B). However, the major headspace compound produced by SlMKS2-expressing cells eluted slightly later than 2TD (peak labeled “1” in FIG. 8B, and also present at lower levels in the chromatograph in FIG. 8A). Mass spectrometry (MS) analysis suggested that it is a 2-tridecenone but the position of the double bond has not yet been determined.

Developmental and Biochemical Connection in MK Synthesis

One of the most surprising findings was the tight relationship between the shape of the trichomes and MK content (FIG. 2). The round and globular trichome shape of the wild species and its progeny was significantly associated with higher MK content. While this observation suggests that morphology constitutes a general barrier to accumulation of volatile compounds, analysis of other volatile compounds in the F2 population did not support this. For example, the distribution of one of the other major volatiles in the glandular trichomes of PI126449, β-caryophellene, was not correlated with trichome shape. Another possible explanation is that since cuticular waxes are complex mixtures of C20-C34 straight-chain aliphatics derived from very long-chain fatty acids, the diversion of the fatty acid pool towards MK comes at the expense of cuticle biosynthesis. This is also supported by the three-way relationship between MK content, trichome shape and the genotype of MKS1 in the segregating population (FIG. 2 and FIG. 3). However, we cannot reject the possibility that the connection between MKS1 variation and trichome shape might be due to genetic linkage with a gene(s) that modulates the development of this specialized organ. The globular shape of the wild-species trichome may be comparable to the “fused” organ morphology seen in mutants with defective cuticle. These fusion phenotypes have been associated with defects in several genes that modulate the biosynthesis and deposition of very-long-chain fatty acids, including enzymes and transporters. Together, these observations suggest a model in which enhanced activity of MK biosynthesis in the wild species may underlie the diversion of the fatty acids to MK at the expense of the synthesis of very-long-chain fatty acids, hence changing the morphology of the trichomes.

The Genetic Basis for MK Biosynthesis in S. habrochaites f glabratum

Although MKs have been found in several plant lineages, their occurrence in S. habrochaites f glabratum is unique in the Solanum genus, suggesting a monophyletic evolution of the specialized metabolic pathway in this subspecies. This study was aimed at identifying the genetic network required for the operation of this pathway within a single cell type, the glandular trichome. The quantitative mode of inheritance of MK in the F2 population (FIG. 1) indicates that several genes are involved in the biosynthesis of these compounds, and that most of the wild-species alleles are recessive, as reflected in the BC population (FIG. 1).

The multiple regression analysis showed that the S. habrochaites f glabratum alleles of genes encoding the first enzyme in the fatty acid biosynthesis pathway, ACC, as well as the enzyme MKS1, are both positively associated with MK biosynthesis. In contrast, other genes encoding enzymes that catalyze intermediate steps did not show this positive correlation. The analysis used in this study for associating candidate genes with MK variation has a few shortcomings that stem from the population structure and the lack of additional genetic markers. The availability of additional markers would have strengthened the association of the variation of MK with the candidate genes, and reduce the possibility that such associations are the result of linkage disequilibrium (LD) with other causative linked and non-linked loci. The fact that all the genes that were included in the multiple regression analysis were also differentially expressed in the two species (FIG. 4C, FIG. 6D), and the F2 population genotyped and phenotyped in this study is relatively large, support the conclusion that a major portion of the MK variation observed in this interspecific population can indeed be attributed to diversity in these genes rather than to other genes that may be in LD with these candidates. Overall, it appears that the flux in MK pathway is controlled at the gene-expression level and the alleles from both species encode almost identical proteins that are likely to be equally active.

Interestingly, the wild-type allele of the gene encoding MaCoA-ACP trans, the enzyme that acts immediately after ACC, was inversely associated with MK content. The relative contribution of this locus to the chemical variation was very low (FIG. 6), and our genetic analysis indicated that the genes encoding these two enzymes are tightly linked (8.8 cM) on chromosome 1. Since these loci act in repulsion, i.e. in the first locus the wild species-allele increases MK content and in the other the wild species-allele reduces it, the results depicted in FIG. 6 may be somewhat biased. The magnitude of the positive and negative additive effects of ACC and MaCoA-ACP trans loci on MK content are likely to be higher due to linkage drag which is not included in a single-point analysis such as that conducted in this study.

The combination of a classical genetic approach (bulked segregant analysis) and transcriptome analysis of the glandular trichomes has led to the discovery of a new participant in MK accumulation in these specialized cells: MKS2. Interestingly, the microarray transcriptome analysis did not detect differences in MKS1 expression levels between bulked high- and low-MK containing F2 plants. Genotyping the individual members of the two groups of five plants explains this unexpected results: four plants from the low-MK bulk carried the wild allele at this locus (ShMKS1) and three of them were homozygous giving a total dosage of seven ShMKS1 alleles. Similar dosages of ShMKS1 alleles were found in the high MK bulk in two heterozygous and three homozygous plants giving a total of eight alleles and leading to equivalent transcripts in both bulks. Similarly, plants that accumulate no MK showed high levels of MKS1 protein in immunoblot test of an F2 population segregating for MK content. This indicates that the regulation of MKS1 is in cis rather than in trans (expression controlled by the locus itself and not by other unlinked factors). This conclusion is also supported by the fact that the wild species' promoter can drive high levels of GFP and GUS expression in glands of the cultivated tomato. In addition, these plants provided a specific and strong demonstration of the epistatic relationship occurring between MKS1 and MKS2 (FIG. 7). Four out of five low-MK plants that carried the ShMKS1 allele were found to be homozygous for the cultivated allele at the MKS2 locus (SlMKS2), thus lacking the wild-species allele ShMKS2. The fifth plant, on the other hand, presented the opposite pattern, i.e. heterozygous at the MKS2 locus but homozygous for the cultivated allele at the MKS1 locus (SlMKS1), thus lacking the wild-species allele ShMKS1. Conversely, analysis of the two-locus haplotype in the high-MK bulk plants showed that they carried at least one wild allele at both the MKS1 and MKS2 loci (ShMKS1 and ShMKS2).

The Role of MKS1 and MKS2 in MK Biosynthesis

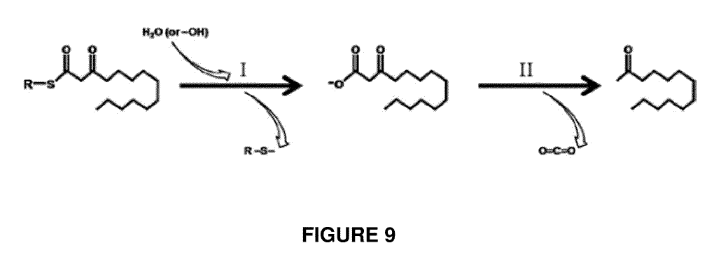

The protein with the highest sequence similarity to MKS2 that has established enzymatic activity is 4-hydroxybenzoyl-coenzyme A thioesterase from Pseudomonas sp. strain CBS3. Indeed, the crystallized 1Z54 and 4HBT crystal structures reveal similar homotetrameric assemblies, which our MKS2 model also reflects (FIG. 12). The conservation of the 4HBT catalytic Asp17 by ShMKS2 and SlMKS2 suggests that they are likely also thioesterases. Moreover, the production of MK in E. coli cells expressing either allele of MKS2 is a strong indication that the heterologous MKS2 enzyme may be capable of hydrolyzing (FIG. 9; step I) and perhaps also decarboxylating (FIG. 9; step 11) 3-ketoacyl intermediates, analogous to the reaction catalyzed by MKS1. However, the production of MK in E. coli expressing MKS2 is not informative in regard to the specific substrates (3-ketoacyl-ACPs or 3-ketoacyl-CoA) because E. coli cells produce both types of substrates.

Proteins with high levels of identity to tomato MKS2 are found throughout the plant kingdom, but interestingly all such sequences outside Solanaceae contain an N-terminal extension predicted to be a plastid or mitochondrial transit sequence (FIG. 5). The SlMKS2 and ShMKS2 (and also a petunia MKS2 homolog) lack such a transit peptide, raising the possibility that these Solanaceae proteins are not localized in the plastids and their substrates may therefore not be 3-ketoacyl-ACPs but rather a 3-ketoacyl-CoAs. The MKS2 proteins, however, do not contain any other obvious subcellular targeting sequences (e.g., no obvious PTS1 or PTS2 sequences that would target the protein to the peroxisomes).

The presence of two distinct enzymes that contribute to the production of the same compound in the same organ, and even in the same cell, is not unprecedented and such functional redundancy was recently reported for eugenol biosynthesis in Clarkia and Petunia (Koeduka et al., 2009). In the case of MKS1 and MKS2, however, genetic evidence for epistatic interactions between the two loci suggests that they do not act independently of each other. Accordingly, MKS1 and MKS2 may potentially form a complex. However, if such a complex is formed, each type of subunit may therefore carry out both reactions of thioester bond hydrolysis and decarboxylation (FIG. 9; steps I and II), or alternatively each subunit may catalyze only one of these reactions.

Alternatively, epistatic interactions may indicate not a physical interaction but that they act sequentially in the pathway from 3-ketoacyl intermediates to methylketones. A closer analysis of the genetic data reveals that although the two wild-species alleles in MKS1 and MKS2 loci are required for the accumulation of high 2TD levels in tomato, some levels are nevertheless found in plants that carry only the MKS2 wild allele (ShMKS2), but not vice versa (FIG. 7). This presents a model for MK biosynthesis in the trichomes in which MKS2 works upstream to MKS1. By this model, MKS2 hydrolyzes the 3-ketoacyl intermediates (FIG. 9; step I), and a low level of spontaneous decarboxylation (FIG. 9; step 11) can occur to produce MK, a step that can be sped-up by MKS1 when present.

These results demonstrate the complex monophyletic evolution of a specialized pathway, and highlight the power of incorporating morphological and chemical data for a detailed understanding of pathways that appear to be isolated in specialized cells. The combined data provide a framework for determining the molecular and biochemical bases for the unexpected relationships between shape and content of the glandular trichomes. Moreover, the genetic and biochemical relationship between MKS1 and newly identified MKS2 loci highlights the major role of epistasis interactions in determining phenotypic variation among populations, and emphasizes the importance of taking it into account when dissecting the genetic basis of complex phenotypes.

As noted, many plants develop glandular trichomes, or appendages, on their aerial parts that synthesize and store specialized (secondary) metabolites involved in plant defense. Plants in the Solanaceae family exhibit a particularly wide range of different types of glandular trichomes, each with its own repertoire of specialized compounds that also varies across species. This chemodiversity is particularly pronounced in the genus Solanum. For example, Type VI glands in Solanum lycopersicum (cultivated tomato) produce mostly terpenes, while the Type VI glands of S. habrochaites subspecies glabratum produce high levels of methylketones (up to 8 mg/g leaf fresh weight) consisting mostly of 2-tridecanone and 2-undecanone.

The present disclosure has elucidated several aspects of the biosynthetic pathways relating to methylketones. It is established that 3-ketoacids are somewhat unstable and can readily undergo decarboxylation when subjected to high temperature and/or non-physiological pHs; a low-level spontaneous decarboxylation occurs under milder conditions. Decarboxylation of 3-keto fatty acids could thus give rise to straight-chain methylketones such as those found in the S. habrochaites glands (FIG. 13). In plants, 3-keto fatty acids could themselves be derived from the hydrolysis of either 3-ketoacyl-ACPs, which are intermediates in the fatty acid biosynthetic pathway of chloroplasts, or could be derived from 3-ketoacyl-CoAs, which are intermediates in the degradation of fatty acids in the peroxisomes (FIG. 13).

As described, initial analysis of a Type VI-specific EST database from a methylketone-producing line of S. habrochaites glabratum (accession PI126449) for highly expressed genes, followed by comparative gene expression analysis (using S. habrochaites accessions with varying amounts of methylketones), identified the gene Methylketone Synthase 1 (ShMKS1) whose expression level positively correlated with high levels of methylketone formation. The 265-residue long protein encoded by ShMKS1 belongs to the α/β-hydrolase superfamily of proteins. Although ShMKS1 does not have a cleavable N-terminal transit peptide, chloroplast import experiments indicated that it could be transported into this organelle. Initially, using in vitro biochemical assays, recombinant ShMKS1 appeared to catalyze the conversion of 3-ketomyristoyl-ACP, an intermediate in fatty acid biosynthesis in the chloroplasts, to 2-tridecanone, suggesting that ShMKS1 possesses both thioesterase and decarboxylase activities that sequentially remove the ACP moiety and decarboxylate the 3-ketomyristic acid intermediate. However, it was noted that the in vitro rate of production of 2-tridecanone from 3-ketomyristoyl-ACP using ShMKS1 was extremely slow.

Genetic and genomic analyses have identified additional genes associated with the high-level production of methylketones in S. habrochaites. These results validated earlier genetic analysis that concluded that methylketone production had a polygenic basis, which explains why it has proven difficult to breed cultivated tomato lines that produce high levels of methylketones in their trichomes. Some of the loci identified encode fatty acid biosynthetic enzymes, a result that is consistent with the need to increase the flux in fatty acid anabolism that provides, directly or indirectly, the substrates for methylketone biosynthesis. Another locus, designated mks2, as disclosed herein, encodes a protein with homology (but <15% identity) to a 4-hydroxybenzoyl-CoA thioesterase (4HBT), a protein belonging to the “hot-dog fold” family, from a Pseudomonas bacterium. Our analysis indicates that high level expression of the S. habrochaites glabratum gene, ShMKS2, in the glandular trichomes was required for high level production of methylketones. Evolutionarily related proteins are encoded in the genomes of various plants, but no functions have yet been assigned to any such plant proteins.

Genetic analysis of an interspecific F2 population between the cultivated and wild species identified significant epistatic interaction between the mks] and mks2 loci. Plants lacking the ShMKS2 allele failed to accumulate any methylketones regardless of the allelic state of the mks1 locus, while absence of the ShMKS1 allele resulted in significantly reduced levels of methylketones. This raised the possibility that the MKS2 protein acts upstream of MKS1 in the pathway for methylketone biosynthesis. Furthermore, expression of ShMKS2 cDNA in E. coli cells resulted in the production of 2-tridecanone, 2-undecanone, and several other methylketones. These genetic and biochemical observations raised the question as to what specific catalytic role ShMKS2 plays in the biosynthesis of methylketones in wild tomato trichomes, and whether it works in parallel or in tandem with ShMKS1. Here we show that ShMKS2 catalyzes the hydrolysis of the 3-keto acyl ACP thioester bond, and ShMKS1 catalyzes the subsequent decarboxylation of the released 3-keto fatty acid, during methylketone biosynthesis.

Genes encoding MKS1 in Solanum lycopersicum and S. habrochaites glabratum.

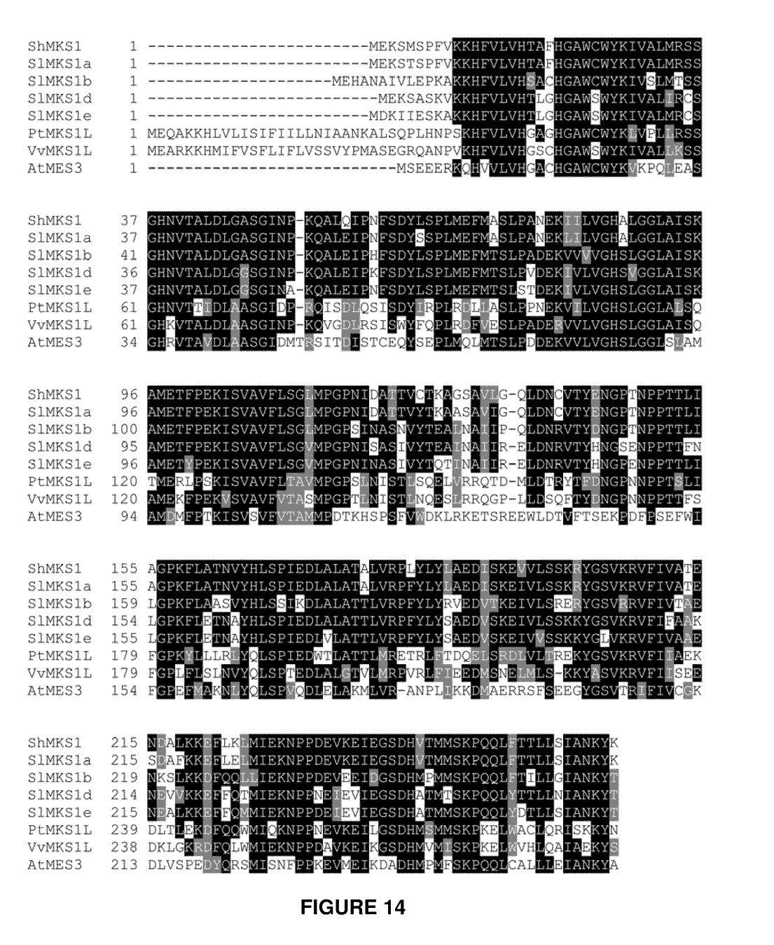

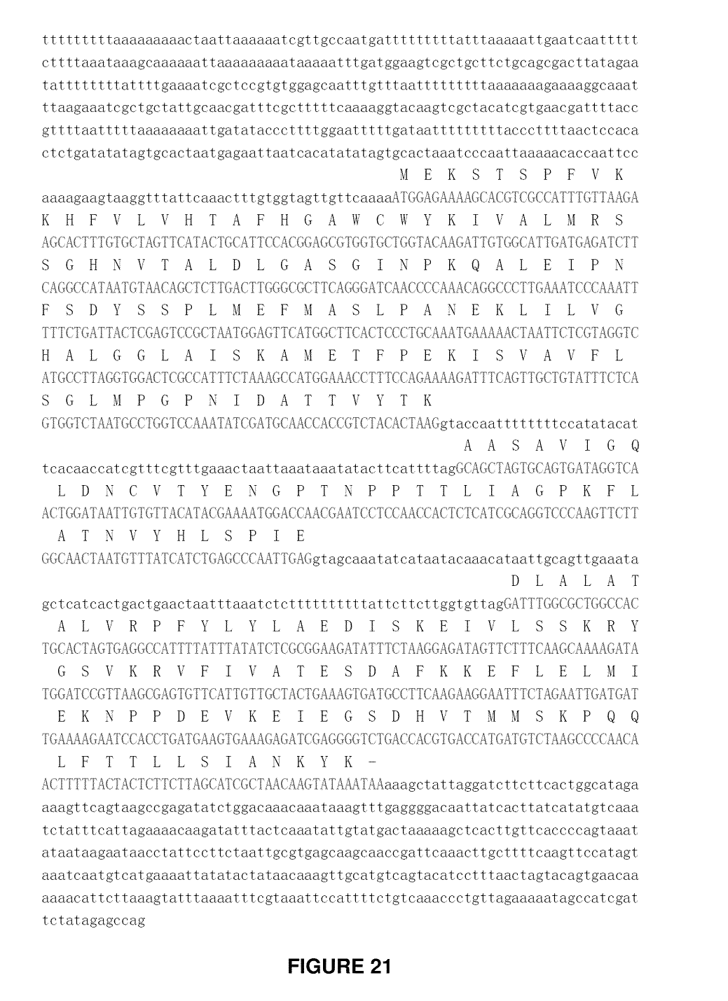

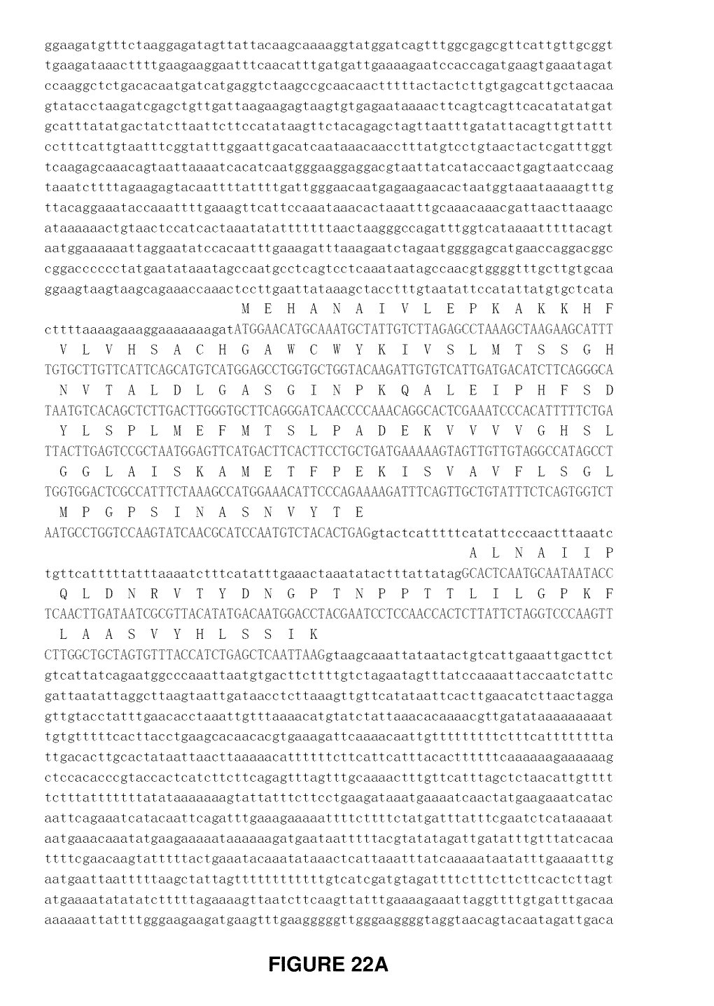

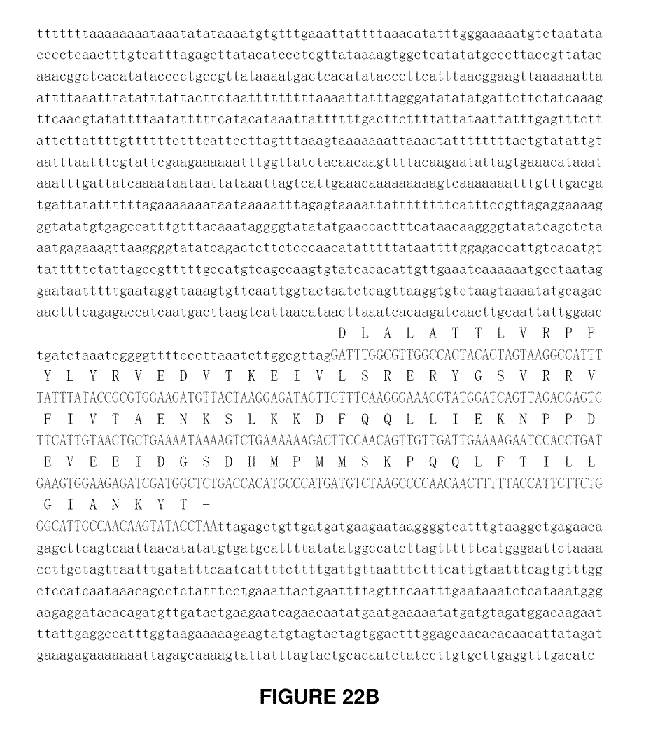

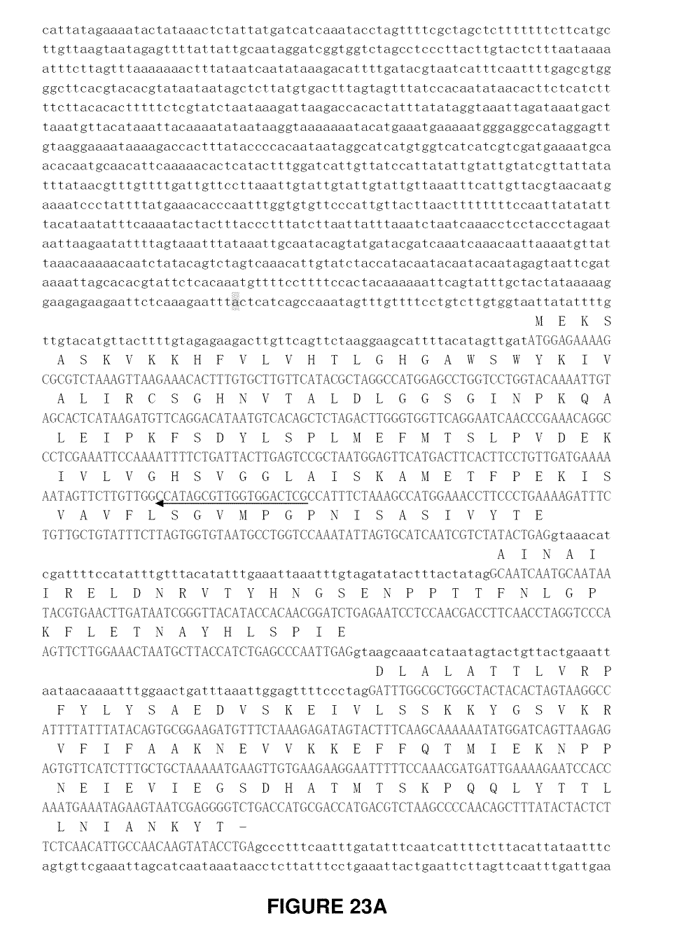

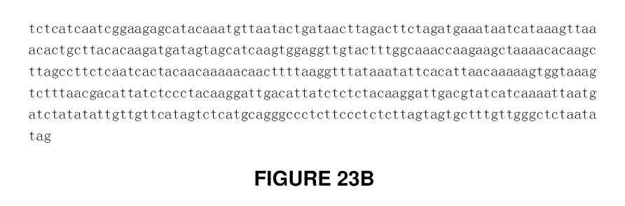

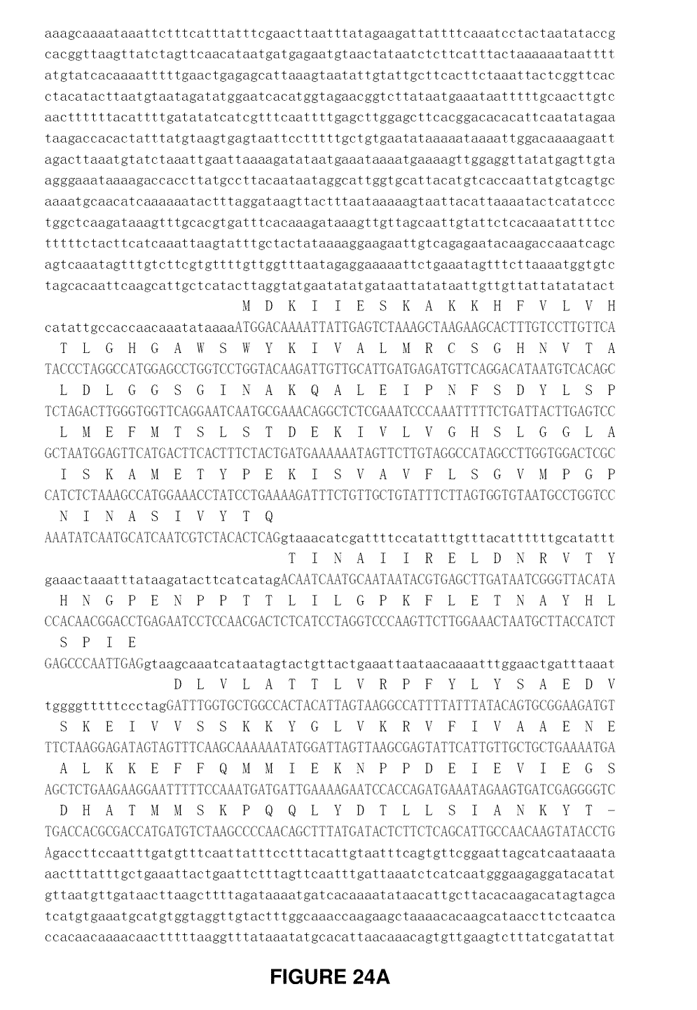

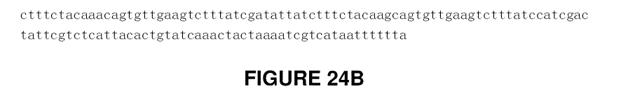

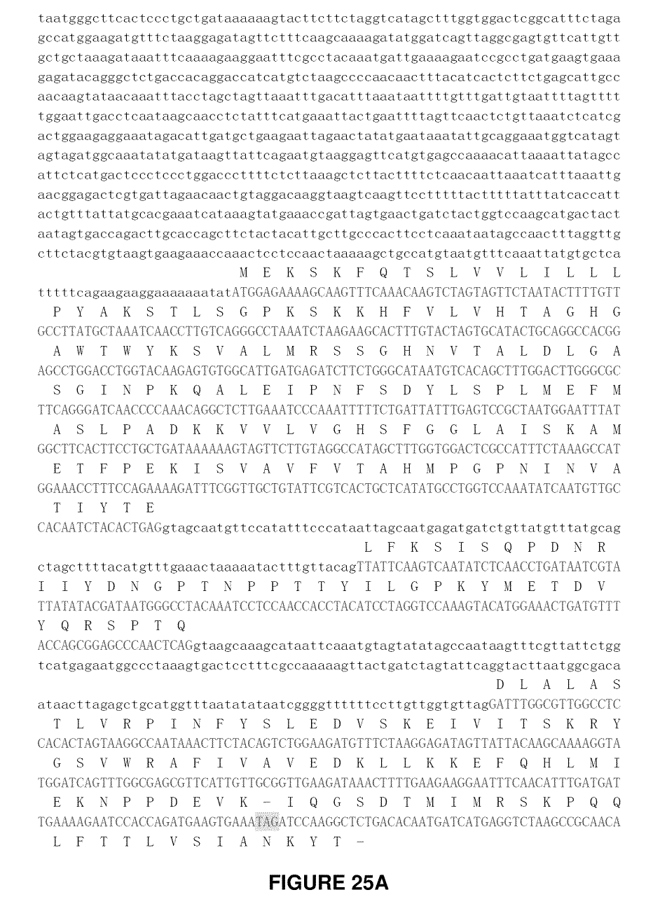

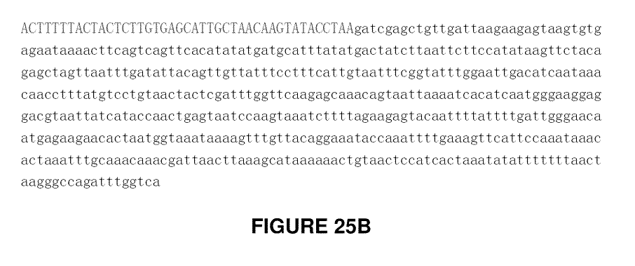

Data mining of the genomic “scaffolds” of S. lycopersicum (available online at solgenomics.net/) indicated that its genome includes at least four genes on two scaffolds, 05390 and 05477, encoding proteins of 264-283 amino acids in length that are >75% identical to ShMKS1. We designated these genes SlMKS1a, SlMKS1b, SlMKS1d and SlMKS1e (FIGS. 21-24); a gene designated as SlMKS1c on scaffold 05477 appears to be a non-functional gene because it contains a premature stop codon (FIG. 25). SlMKS1a is the most similar gene to ShMKS1, encoding a protein with 95% identity to ShMKS1 (FIG. 14). Proteins with similar size that are approximately 54% identical to ShMKS1 have recently been found in the genome of poplar and grape (FIG. 14), although their functions are presently unknown. However, the most similar protein encoded by a gene in the Arabidopsis genome, AtMES3 (a protein capable of hydrolyzing methyl IAA and methyljasmonate), is only 40% identical to ShMKS1 (FIG. 14). As reported for ShMKS1, the N-terminal region of all these newly identified S. lycopersicum MKS1 proteins, as well as the analogous region within the closely related homologs from other species, does not appear to constitute amino-terminal extensions that could function as cleavable transit peptides.

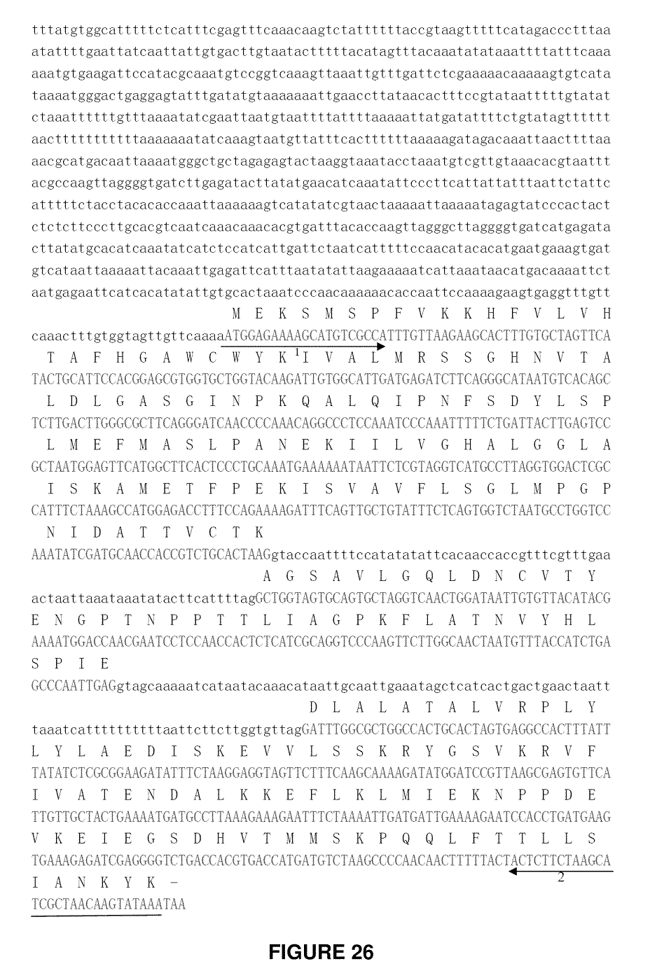

Interestingly, while SlMKS1a is the most similar gene to ShMKS1, only one cDNA for it was found in the NCBI database, consistent with its low expression level. Moreover, no cDNAs/ESTs were found for SlMKS1b while a small number of ESTs for SlMKS1d and SlMKS1e were observed, the majority of which were obtained from trichomes. We used oligonucleotide primers encoding the beginning and end of the coding region of ShMKS1 in PCR experiments with genomic DNA to isolate the DNA fragment containing all exons and introns of this gene (FIG. 26). The number and positions of introns in ShMKS1 were found to be the same as those found in the S. lycopersicum MKS1 genes.

Genes Encoding MKS2 in S. lycopersicum and S. habrochaites glabratum

We determined that the longest available ShMKS2 cDNA contained an open reading frame, starting with a Met codon (ATG), of 149 codons. In addition, we showed that the protein encoded by this cDNA was highly similar (>70% identity across the equivalent region) to putative, functionally uncharacterized proteins from numerous plant species, including four from Arabidopsis thaliana, as well as showing limited similarity (<15% identity) to 4HBT from Pseudomonas sp. Based on this ShMKS2 cDNA sequence and analysis of homologous ESTs from S. lycopersicum available at the time, the orthologous S. lycopersicum gene was deemed to encode a protein similar in size to that of ShMKS2, and consequently a cDNA was isolated by RT-PCR from S. lycopersicum and named SlMKS2. Protein sequence comparisons indicated that the homologous proteins from all other plant species, with the exception of the Solanaceae proteins, have an N-terminal extension that was predicted to function as a transit sequence to direct the protein into the plastids.

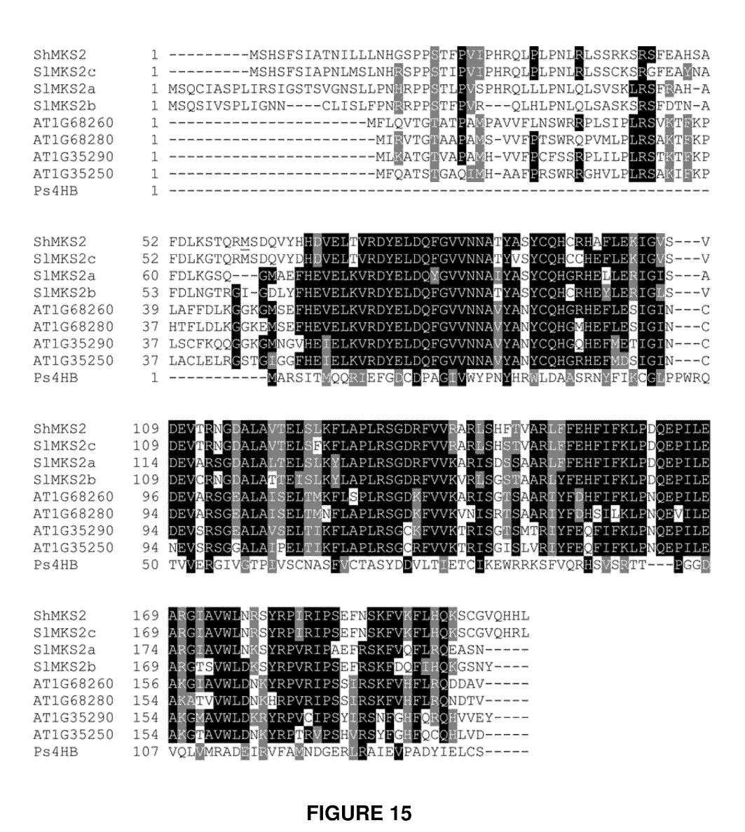

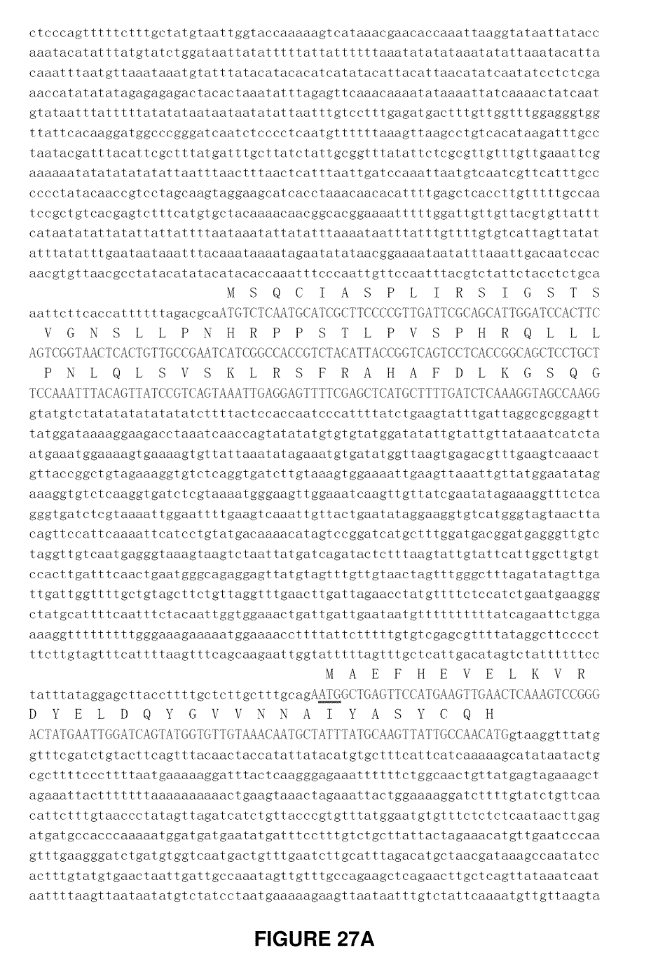

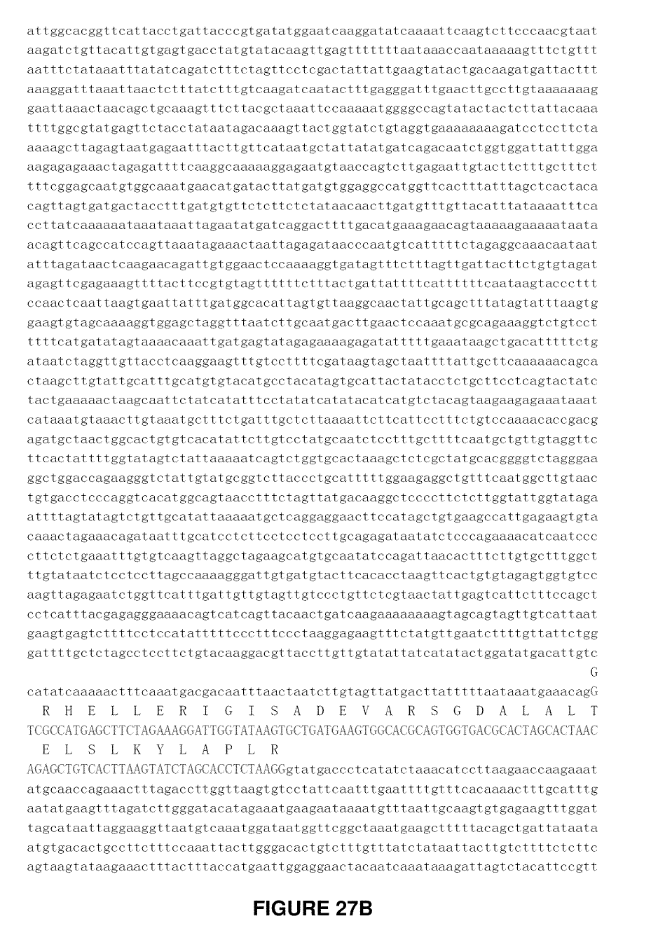

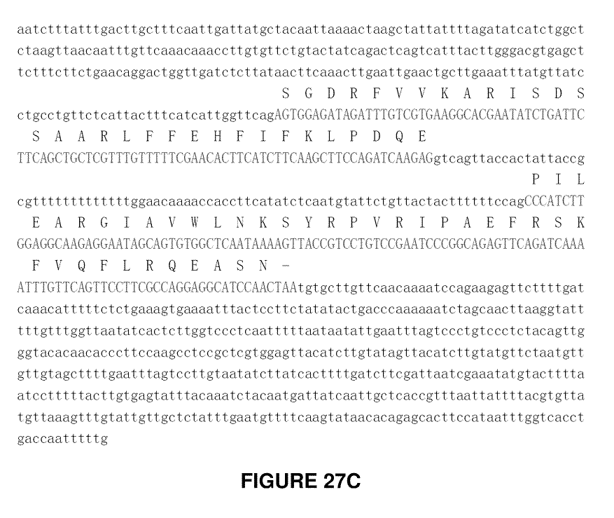

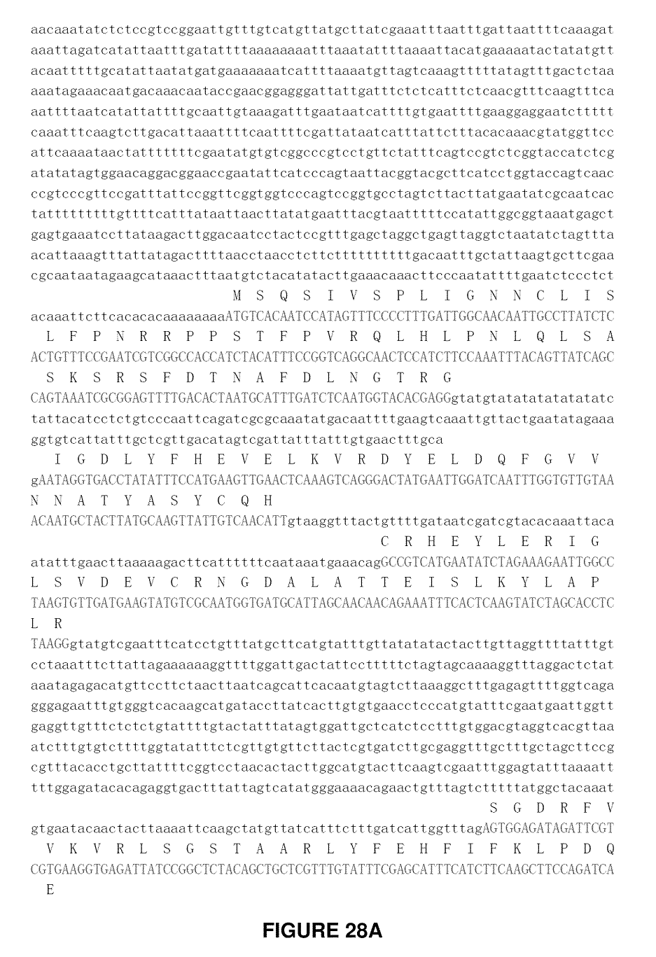

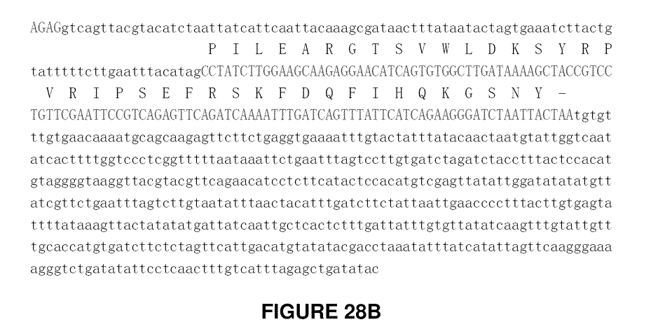

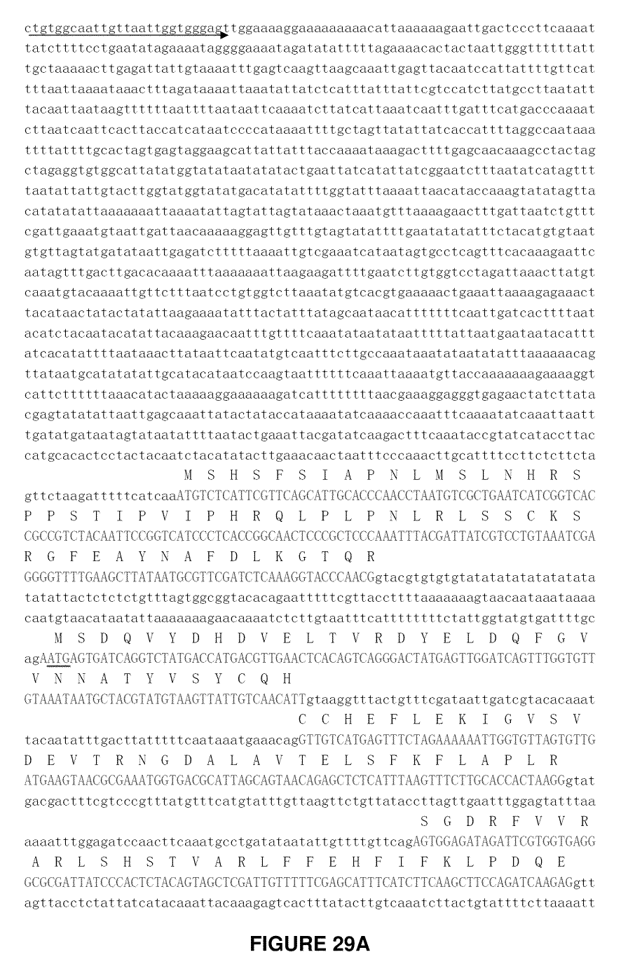

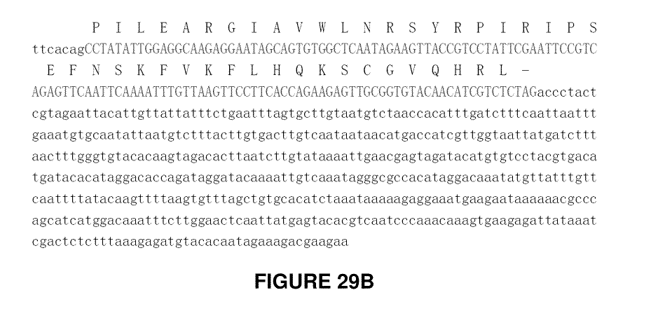

Mining the S. lycopersicum genome resulted in the identification of three genes on the same “scaffold” (Scaffold 04161) that encode proteins with >90% identity (within the equivalent region) to previously reported ShMKS2 sequences; we named these genes SlMKS2a, SlMKS2b, and SlMKS2c (FIG. 15 and FIGS. 27-29). The EST databases contain ESTs for SlMKS2a and SlMKS2b, but not for SlMKS2c. Consistent with this observation, our previously reported SlMKS2 cDNA is derived from SlMKS2a, although SlMKS2c encodes a protein with a higher identity to ShMKS2 (95%). All three of these S. lycoperiscum MKS2 genes have 5 exons and 4 introns (whose positions are conserved in comparison to the intron positions in the homologous A. thaliana genes). By comparing the sequence of the SlMKS2 cDNA with the genomic sequence of SlMKS2a, from which it is derived, and the sequence of ShMKS2 cDNA with the genomic sequence of SlMKS2c, to which it is most similar, we noted that the first ATG codon of the open reading frame in each of these cDNAs were equivalent to the ATG codon that occurs in positions 2-4 of exon 2 in the SlMKS2a and SlMKS2c genes (see underlined codon in FIGS. 27 and 29). This suggested that these MKS2 cDNAs from both species were incomplete. Indeed, although no SlMKS2a EST that contains the entire coding region of exon 1 is available, the sequence of one SlMKS2b EST that includes the entire coding region of exon 1 is now in the EST database of NCBI (accession number DB688740).

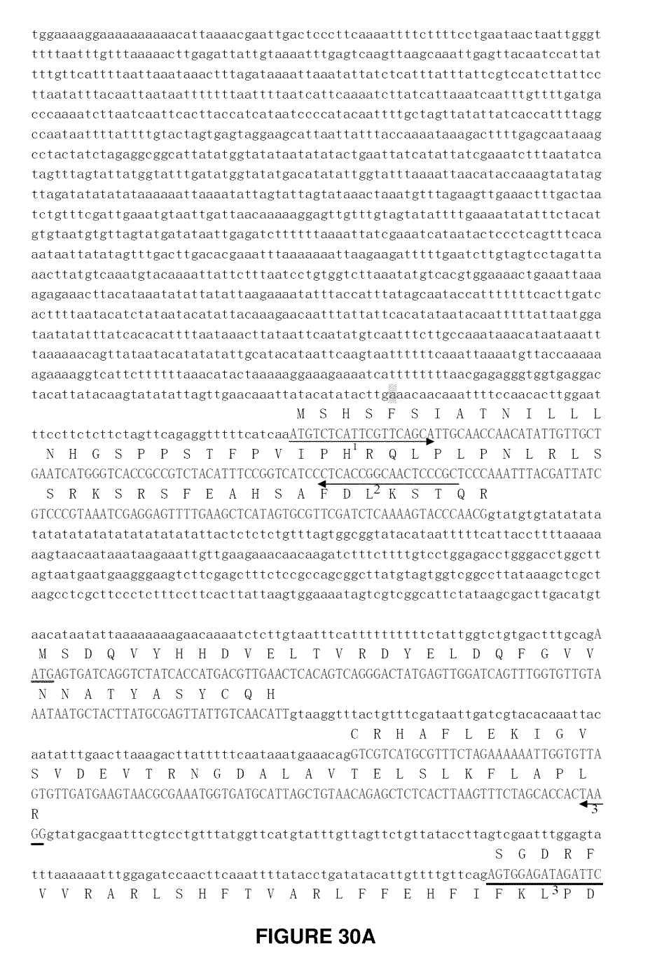

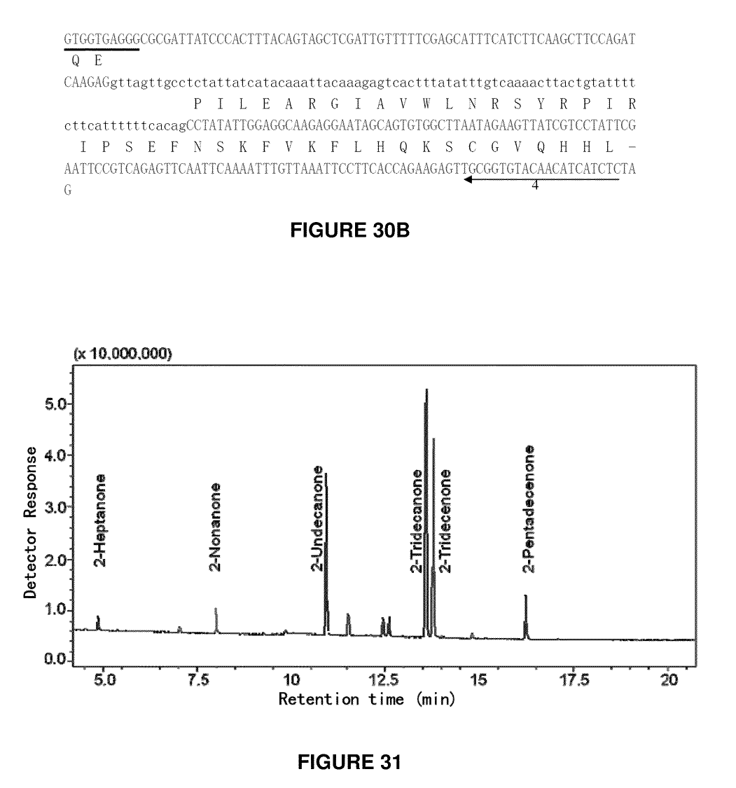

To determine the beginning of the transcript of ShMKS2, two independent 5′RACE experiments were performed using two specific primers complementary to the 3′ end and middle of the coding region, respectively (see FIG. 30). Analysis of the DNA fragments produced in these experiments by agarose gel electrophoresis gave a single sharp band in both cases. The sequence of the resulting fragments from both experiments was determined, and in both cases the sequence obtained indicated that ShMKS2 transcripts are considerably longer at their 5′ ends than was previously seen in the cDNA. This newly uncovered 5′ end sequence, identical in both 5′RACE experiments, included the region homologous to exon 1 in the SlMKS2 genes, which encodes a putative transit peptide, as well as 63 nucleotides of the 5′ UTR (see FIG. 30).

To determine the complete genomic structure of the ShMKS2 gene, we used a forward oligonucleotide primer based on the sequence at the beginning of the coding region in exon 1 of ShMKS2 (as determined by the 5′ RACE experiment) and a reverse primer based on the sequence at the end of the coding region in a PCR experiment with S. habrochaites glabratum genomic DNA, and isolated and characterized the genomic fragment containing ShMKS2 (FIG. 30). Using a homology-based PCR approach, we also isolated a 1.5-kb fragment upstream of exon 1 of ShMKS2 with a forward oligonucleotide primer whose sequence was based on the sequence of the promoter of SlMKS2c (FIG. 29) and a reverse primer derived from the beginning of the ShMKS2 coding region. Analysis of the complete sequence of the ShMKS2 gene (FIG. 30) indicates that its structure, with 5 exons and 4 introns and encoding a protein of 208 amino acid residues with a predicted plastidic transit peptide, is very similar to that of the S. lycopersicum MKS2 genes (FIG. 15).

Subcellular Localization of ShMKS2

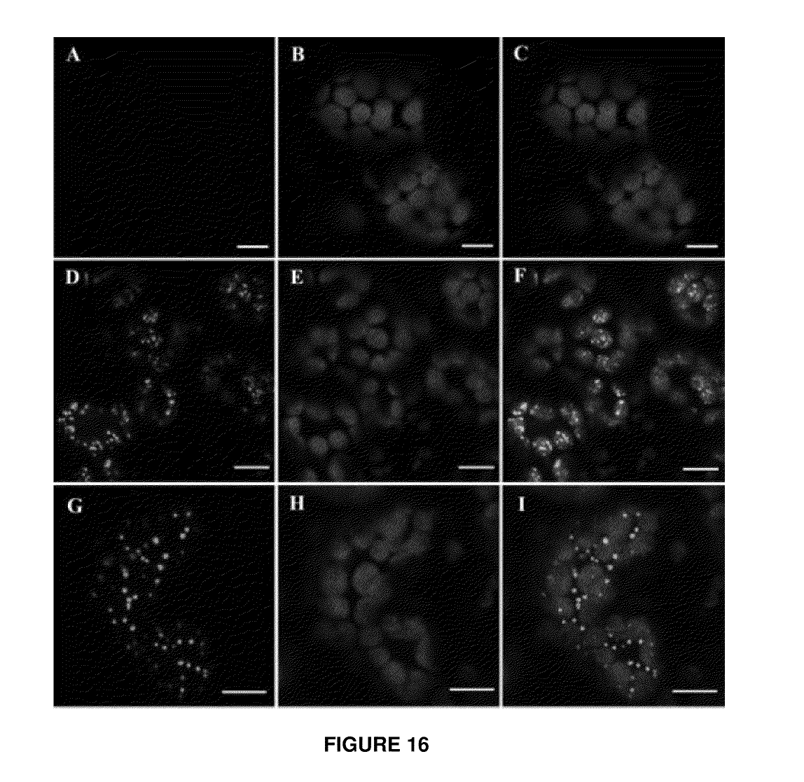

To determine the subcellular localization of ShMKS2 proteins, we injected Nicotiana benthamiana leaves with a solution of Agrobacterium tumefaciens cells carrying various constructs in which the ShMKS2 had been fused to the enhanced green fluorescent protein (eGFP) under the control of the cauliflower mosaic virus 35S promoter, and visualized targeting by confocal microscopy. No green fluorescence was detected in tobacco leaf cells transformed with an empty binary vector (FIG. 16A-C). In the tobacco leaf cells transformed with the full length ShMKS2-eGFP construct, GFP-labeled signals, seen as a punctate pattern, were observed from the same area from which red fluorescence is observed and therefore identified as the chloroplasts (FIG. 16D-F). In tobacco leaf cells transformed with a ShMKS2-eGFP construct that lacked the putative ShMKS2 transit peptide, the green fluorescence dots no longer coincided with chloroplast red fluorescence (FIG. 16G-I).

Expression of ShMKS1 and ShMKS2 in E. coli and Production of Methylketones

We described that analysis of the spent medium of E. coli cells expressing ShMKS2 demonstrated the presence of several methylketones with 2-tridecanone, 2-tridecenone, and 2-undecanone predominating (see Ben-Israel et al. (2009), Multiple biochemical and morphological factors underlie the production of methylketones in tomato trichomes. Plant Physiology 151: 1952-1964). However, in those studies, no attempt was made to measure the production of 3-ketoacids, which are the putative intermediates in the synthesis of the final methylketone products (FIG. 13). Direct measurement of 3-ketoacids is difficult since these compounds are unstable. However, a chemical approach employing sulfuric acid and heat treatment was developed leading to greatly enhanced decarboxylation and conversion of the water-soluble 3-ketoacids into easily extractable methylketones, which can then be directly measured by GC-MS.

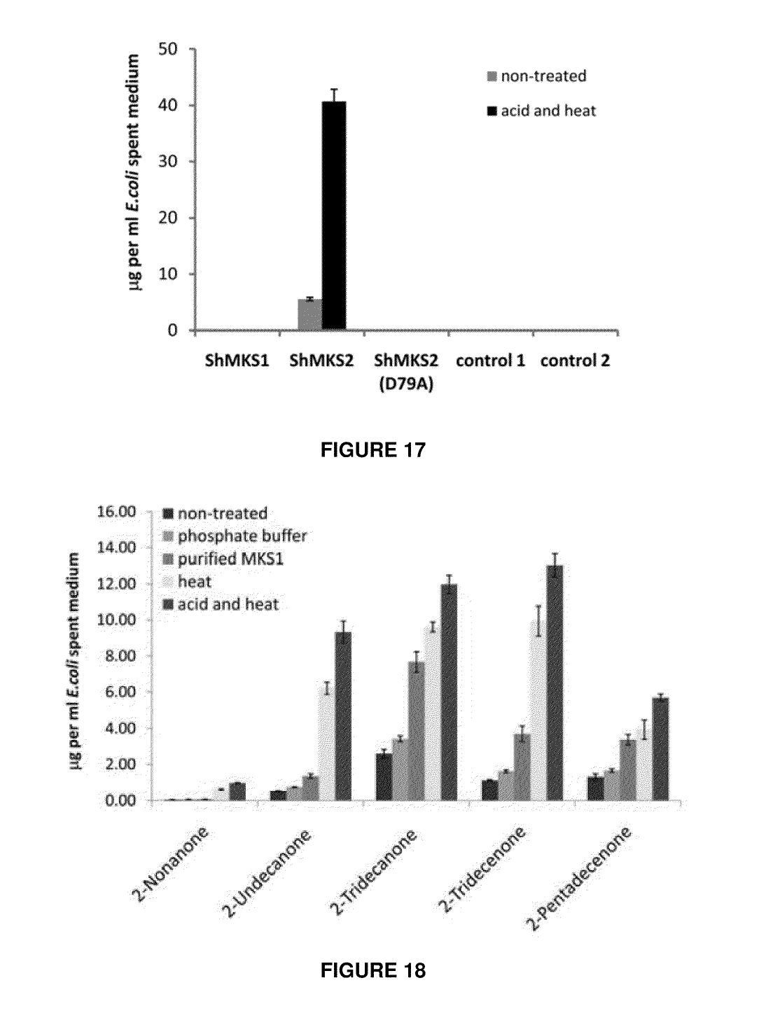

To test if expression of either ShMKS1 or ShMKS2 (without its transit peptide) in E. coli results in the formation of 3-ketoacids, we collected spent media of bacterial cells expressing each of them (by centrifuging out the cells at the end of the incubation period), heated the spent media at 75° C. for 30 min in the presence of 1 M sulfuric acid, extracted with hexane, then injected the hexane fraction in a GC-MS. Spent media of cells expressing either ShMKS1 or a plant gene unrelated to the methylketone biosynthetic pathway, as well as of cells carrying the same vector (pEXP5-CT/TOPO) without an introduced gene, contained no methylketones with or without the acid and heat treatment (FIG. 17). On the other hand, the spent medium of E. coli cells expressing ShMKS2 contained 5.6±0.32 μg/μL of total methylketones, and the amount of methylketones increased 8 fold, to 40.7±2.1 μg/μL, after the spent medium was treated with acid and heat (FIG. 17).

In the bacterial thioesterase 4HBT, the aspartate residue at position 17 was identified as the catalytic residue required for thioester bond cleavage. We mutated the equivalent Asp codon in ShMKS2 (the Asp encoded by codon 79 of the complete open reading frame) to an Ala codon and expressed the mutated gene (without the transit peptide-encoding region) in E. coli. The spent medium of cells expressing this mutant ShMKS2 protein did not contain any methylketones, with or without prior acid and heat treatment (FIG. 17).

A more detailed analysis of the spent medium of E. coli cells expressing ShMKS2 showed that the major compounds in the untreated spent medium were 2-undecanone (0.51 μg/μL), 2-tridecanone (2.6 μg/μL spent medium), 2-tridecenone (1.1 μg/μL) and 2-pentadecenone (1.3 μg/μL) (FIG. 18). Lower amounts of 2-nonanone (0.05 μg/μL) were also detected (FIG. 18). When the spent medium was heated at 75° C. for 30 min in the absence of sulfuric acid, the yield of methylketones increased up to 0.60±0.05 μg/μL 2-nonanone (11.9-fold over non-treated control), 6.22±0.34 μg/μL 2-undecanone (12.2-fold), 9.61±0.27 μg/μL 2-tridecanone (3.7-fold), 9.94±0.83 μg/μL 2-tridecenone (9-fold) and 3.94±0.54 μg/μL 2-pentadecenone (3.0-fold). The yield was increased even further in the combined heat and acid treatment, reaching maximum levels of 0.95±0.04 μg/μL 2-nonanone (19.1-fold over non-treated control), 9.33±0.61 μg/μL 2-undecanone (18.3-fold), 11.99±0.51 μg/μL 2-tridecanone (4.6-fold), 13.04±0.63 μg/μL 2-tridecenone (11.8-fold) and 5.69±0.21 μg/μL 2-pentadecenone (4.4-fold).

When purified ShMKS1 (75 μg/mL in 12.5 mM Na+-phosphate buffer, pH 6.8) was added to the spent medium (3 μg/mL final concentration) and incubated for 2 h prior to hexane extraction, levels of extractable methylketones were significantly higher than in spent medium treated with phosphate buffer alone, although not as high as the levels of methylketones observed after acid and heat treatments. Moreover, the treatment with purified ShMKS1 seemed to favor an increase in 2-tridecanone over other methylketones (FIG. 18).

In Vitro Decarboxylase Activity Assays for ShMKS1 and ShMKS2

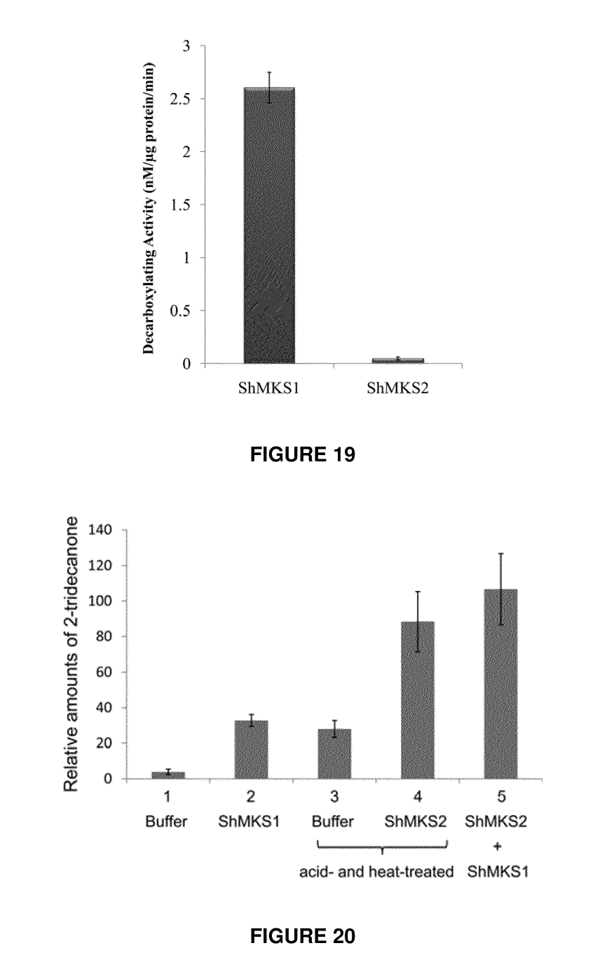

To examine the possible decarboxylase activity of ShMKS1 as well as ShMKS2 in vitro, we tested homogenous recombinant ShMKS1 and partially purified recombinant ShMKS2 proteins (without their transit peptides) for their ability to convert 3-ketomyristic acid into its 2-tridecanone decarboxylated product. Notably, ShMKS1 produced 2.6 nM of 2-tridecanone/m of protein/min, while ShMKS2 showed no decarboxylase activity (FIG. 19). In steady-state kinetic assays, ShMKS1 was determined to have a K_{M} of 18.4±5.6 μM for 3-ketomyristic acid, with an apparent k_{cat} of 227.9±24.1 min⁻¹.

In Vitro Thioesterase Activity Assays for ShMKS1 and ShMKS2

To examine the potential thioesterase activity of ShMKS1 and ShMKS2 in vitro, we added 2.5 μg of each protein to a 500 μL solution of freshly prepared 3-ketomyristoyl-ACP. This protein-linked substrate was prepared using a sequential in vitro enzymatic system involving the addition of multiple starting materials and enzymes and the reaction allowed to proceed for 5 hr (see Examples, Materials, and Methods). Due to the highly unstable nature of 3-ketomyristoyl-ACP, this compound was not further purified but instead the solution in which it was synthesized was used as the “substrate solution” for qualitative in vitro thioesterase activity assays.

Aliquots of this substrate solution were incubated with buffer, ShMKS1, ShMKS2, or both ShMKS1 and ShMKS2 for 30 min at 23° C. Extraction of buffer-incubated substrate solution with hexane, followed by GC-MS analysis, resulted in detection of almost no 2-tridecanone (FIG. 20). However, when the buffer-incubated substrate solution was treated with acid, heated at 75° C., cooled and then extracted with hexane, 2-tridecanone was detected (FIG. 20), indicating that the substrate solution contained free 3-ketomyristic acid in addition to 3-ketomyristoyl-ACP. When the substrate solution was incubated with ShMKS1, and then directly extracted with hexane (i.e., without first treating the sample with acid and heat), the amount of 2-tridecanone obtained was slightly higher, but not significantly so (t test, p=0.062, α=0.05), than the amount found in the buffer-incubated sample treated with acid and heat (FIG. 20). However, when the substrate solution was incubated with ShMKS2, then further treated with acid and heat, the amount of 2-tridecanone formed was approximately 3-fold higher than levels found in buffer-incubated substrate solution treated with acid and heat. Finally, when the substrate solution was co-incubated with both ShMKS1 and ShMKS2 for 30 min, then directly extracted with hexane, the amount of 2-tridecanone was slightly higher than that found in ShMKS2-incubated substrate solution treated with acid and heat, but again not significantly so (t test, p=0.102, α=0.05) (FIG. 20).

Enzymatic Activities of ShMKS1 and ShMKS2

Although we reported that incubation of crude preparations of 3-ketomyristoyl-ACP derived from a complex mixture of fatty acid biosynthetic components with purified ShMKS1 resulted in the appearance of 2-tridecanone, we also noted that the yield of the in vitro reaction was exceedingly low. Here we show that the expression of ShMKS1 in E. coli does not result in the production of methylketones. On the other hand, we confirm and expand on a subsequent finding that methylketones are present in the growth medium of E. coli expressing ShMKS2 (FIG. 18). Furthermore, treatments of this spent medium with acid and heat, or with purified ShMKS1 protein, greatly elevate the levels of methylketones extracted and detected by GC-MS analysis. Since it is well established that treatment with acid and heat, or even heat alone, greatly accelerates decarboxylation of 3-ketoacids to form methylketones, the increase in levels of methylketones extracted from acid- and heat-treated spent medium of E. coli cells expressing ShMKS2 indicates that substantial amounts of 3-ketoacids were present in this spent medium prior to this treatment, and further suggests that ShMKS2 acted as a thioesterase, producing 3-ketoacids from either 3-ketoacyl-CoA or 3-ketoacyl-ACP precursors.

Because treatment of the spent medium of ShMKS2-expressing E. coli cells with purified ShMKS1 also increased the extractable levels of methylketones by several fold without the use of heat or acid, it appears that ShMKS1 possesses decarboxylase activity. This latter activity was later confirmed quantitatively. However, ShMKS1 did not seem to possess a thioesterase activity when expressed in E. coli, since no methylketones could be detected in the spent medium of these cells even after acid and heat treatment, indicating a lack of 3-ketoacids in the spent medium (FIG. 17).

To directly test the enzymatic activities of ShMKS1 and ShMKS2, we carried out in vitro assays. ShMKS1 exhibited decarboxylase activity on 3-ketomyristic acid, while ShMKS2 did not (FIG. 19). When ShMKS1 was added to a solution containing enzymatically synthesized 3-ketomyristoyl-ACP but also some free 3-ketomyristic acid (due to the instability of 3-ketomyristoyl-ACP it could not be further purified), the amount of 2-tridecanone obtained was slightly higher than the amount of 2-tridecanone observed after incubation of the same volume of substrate solution with buffer, followed by acid and heat treatment. However, the difference was not significant, indicating that ShMKS1 possesses little or no in vitro thioesterase activity, a result also consistent with the lack of 3-ketoacids in the spent medium of ShMKS1-expressing E. coli. On the other hand, incubation of the substrate solution with ShMKS2 resulted in a 3-fold higher amount of 2-tridecanone than what would be expected simply from the acid- and heat-induced decarboxylation of the 3-ketomyristic acid present in the solution (FIG. 20). The additional amount of 2-tridecanone most likely resulted from the intrinsic thioesterase activity of ShMKS2 on 3-ketomyristoyl-ACP, leading to the liberation of 3-ketomyristic acid which then underwent chemically mediated decarboxylation in the acid and heat treatment. Furthermore, co-incubation of substrate solution with both ShMKS1 and ShMKS2 resulted in similar amounts to those observed during the incubation with only ShMKS2 followed by acid and heat treatment, indicating that MKS1 was able to decarboxylate the 3-ketomyristic acid that the thioesterase activity of ShMKS2 on 3-ketomyristoyl-ACP produced.

Since the in vitro assays indicate that ShMKS2 lacks decarboxylase activity, the small amount of methylketones (relative to the corresponding 3-ketoacids) found in the spent medium of ShMKS2-expressing E. coli cells (FIG. 18) was thus most likely due to slow non-enzymatic decarboxylation of the 3-ketoacids released by ShMKS2 during the overnight incubation period.

Taken together, these data suggest that in tomato trichomes, ShMKS2 and ShMKS1 work sequentially, ShMKS2 first liberating 3-ketoacids and ShMKS1 catalyzing their decarboxylation to produce the final methylketone products. This is consistent with the observations that several-fold more methylketones are found in the tomato trichomes when ShMKS2 is highly expressed and ShMKS1 is expressed at low levels than in the opposite case when ShMKS1 is expressed at high levels but ShMKS2 is expressed at low levels. This is easily rationalized since ShMKS2 activity is required for production of 3-ketoacids while ShMKS1 activity is limited to decarboxylation, and some decarboxylation may occur spontaneously both in planta and in E. coli given sufficient time (although ShMKS1 activity substantially increases methylketone production in both cases). Our earlier observation that small amounts of 2-tridecanone were produced in vitro when a solution containing enzymatically prepared 3-ketomyristoyl-ACP was incubated with ShMKS1 can now be explained to be the result of the decarboxylating activity of ShMKS1 on the free 3-ketomyristic acid present in the substrate solution, as shown in the present study (FIG. 20).

Mature ShMKS2 Localizes to the Plastid and Catalyzes 3-ketoacyl-ACP Hydrolysis

The results of transient expression of ShMKS2-GFP fusion protein in tobacco leaves indicated that the ShMKS2 protein localized to the plastids, as has been shown for ShMKS1 by in vitro chloroplast import studies. A plastidic localization is consistent with the hypothesis that ShMKS2 works by competing for 3-ketoacyl-ACP intermediates formed iteratively during fatty acid elongation in the plastids, rather than from 3-ketoacyl-CoA intermediates formed catabolically during fatty acid degradation in peroxisomes. This conclusion is consistent with the demonstrated in vitro thioesterase activity of ShMKS2 with 3-ketomyristoyl-ACP, although 3-ketoacyl-CoA could not be obtained for comparison purposes.

The range of methylketones produced by expressing ShMKS2 in E. coli was very similar to that seen in S. habrochaites glabratum trichomes, with 2-tridecanone and 2-undecanone being the most abundant, suggesting that ShMKS2 displays a preference for similar chain-length intermediates in both plants and E. coli. Intriguingly, substantial amounts of 2-tridecenone (with one double bond present between C3 and C4) and some 2-pentadecenone (also with one double bond present between C3 and C4) were also observed in ShMKS2-expressing E. coli cultures (FIG. 18), whereas these compounds were not detectable in S. habrochaites glabratum trichomes. The position of the double bond indicates that ShMKS2 is able to act on 2-oxo-4-en acyl-ACPs that are at least 14 carbons long. Such intermediates could have resulted from the elongation of un-reduced 2-en acyl-ACPs. Whether ShMKS2 acts on such intermediates in S. habrochaites glabratum trichomes or whether this activity is simply a peculiarity of its heterologous expression in E. coli is not presently known.

Evolution of ShMKS1 and ShMKS2

Low levels of methylketones have occasionally been found in plant species from diverse taxa outside the genus Solanum, but their mode of synthesis has not yet been determined. In Solanum, only S. habrochaites glabratum has been reported to synthesize and store high levels of methylketones (up to 8 mg/leaf FW) in their Type VI glandular trichomes, while the trichomes of the cultivated tomato (S. lycopersicum) contain methylketones at levels that are about 1.000-fold lower. We also showed that the expression of both SlMKS1 and SlMKS2 genes in S. lycoperiscum trichomes is considerably lower than in their related wild species. The presence of proteins in species outside Solanum with homology to MKS1 and MKS2 raises the question of whether such proteins are involved in methylketone biosynthesis, albeit at very low rates, and if not, how the Solanum MKS1 and MKS2 proteins cooperatively acquired the catalytic ability to biosynthesize considerable amounts of methylketones.

It is possible that regardless of the original function of MKS2-like genes, simply increasing the expression of such a gene possessing a low level of an alternative activity (with a concomitant increase in fatty acid biosynthetic flux) will lead to production of some methylketones (for example, high-level expression in E. coli of the ShMKS2 homologs from Arabidopsis thaliana also leads to substantial methylketone production; FIG. 31). The ability to produce methylketones, with their insecticidal properties, would then be positively selected. However, overexpression of a ShMKS2-type protein without a the presence of a dedicated decarboxylase will also lead to accumulation of the 3-ketoacids intermediates, which could interfere with fatty acid biosynthesis, and perhaps the ancestral MKS1 already possessing low level 3-ketoacid decarboxylation activity was selected because it conferred an advantage to the plant by decomposing such acids and increase the production of methylketones.

It is interesting to note that unlike MKS2-like proteins from A. thaliana, which catalyze a similar reaction to ShMKS2 in E. coli, ShMKS1 and its ortholog SlMKS1a, are fundamentally different from the other SlMKS1 and MKS1-like proteins in other species, in that the first two are missing what at first glance appears to be a catalytically essential Ser (at position 87 in ShMKS1), part of the catalytic triad necessary for the α/β-hydrolase activity of many proteins in the α/β-hydrolase superfamily. Although ShMKS1 and SlMKS1a clearly belong to this family, they have an Ala substitution at this position and are therefore unlikely to possess hydrolase activity. It thus appears that a bona fide MKS1 evolved recently in the Solanum lineage by acquiring decarboxylase activity and attenuating its more ancient hydrolase activity.

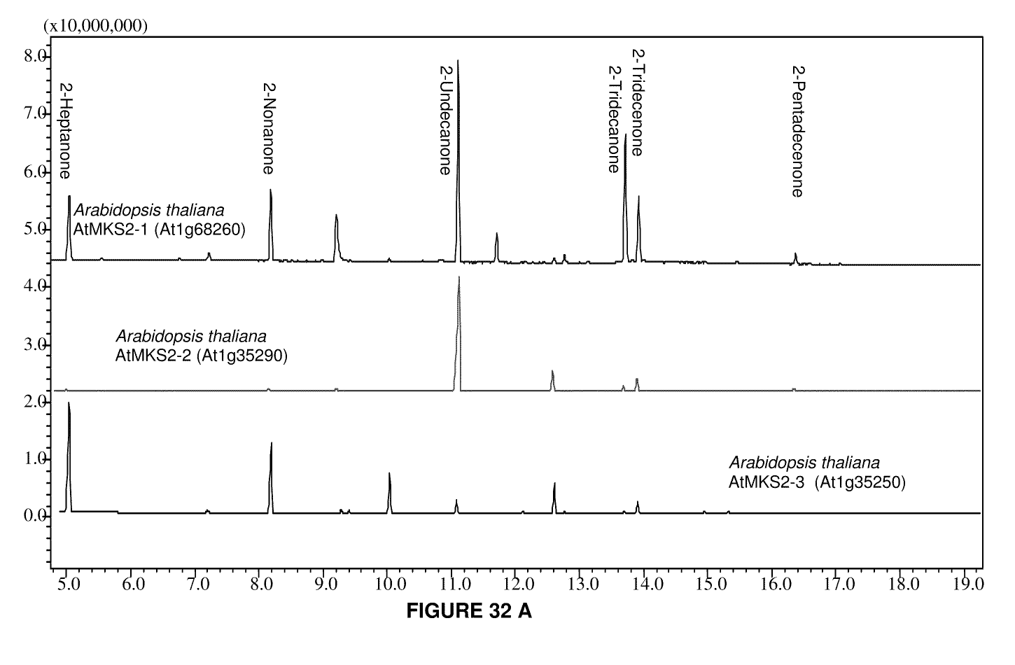

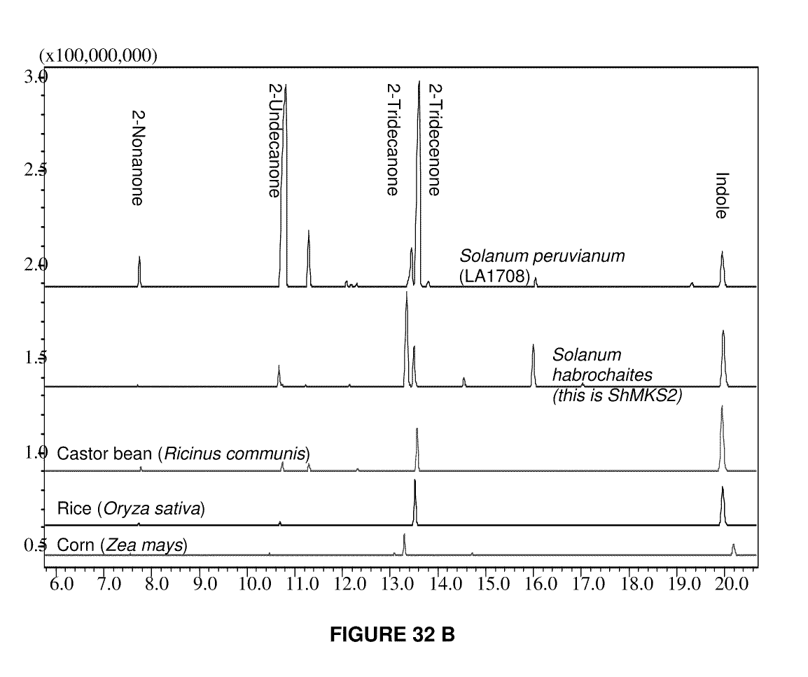

The present technology further includes additional MKS2 genes, including three genes from Arabidopsis (At) and genes from rice, corn, castor bean, and Solanum peruvianum. These MKS2 genes include: AtMKS2-1 (gene number At1g68260) (SEQ ID NO: 76); AtMKS2-2 (gene number At1g35290) (SEQ ID NO: 77); AtMKS2-3 (gene number At1g35250) (SEQ ID NO: 78); Rice (Oryza sativa) Accession Number CAE01692.2 (SEQ ID NO: 79); Corn (Zea mays) Accession Number ACR38219.1 (SEQ ID NO: 80); Castor bean (Ricinus communis) Accession Number XP_{—}002526988.1 (SEQ ID NO: 81); and LA1708 (Solanum peruvianum) no accession number (SEQ ID NO: 82). These genes have been tested for activity in E. coli and gas chromatography data with identification of the products is shown in FIGS. 32 A & B. Gas chromatography was used to separate the products produced in E. coli after expression of the MKS2 genes from the indicated species. Products were identified by mass spectrometry.

Accordingly, the present technology provides the enzyme Methylketone Synthase 2, which is involved in the fatty acid biosynthetic pathway converting β-ketoacyl intermediates to methylketones of varying lengths ranging from C₇ to C₂₀. In some embodiments, the methylketone synthase is MKS2 (ShMKS2, GenBank Accession No. ACG63705.1 GI:195979085) derived from Solanum habrochaites (also known as Solanum habrochaites and Lycopersicon hirsutum f glabratum (Deposit Voucher Accession No. PI 126449). In some embodiments, the MKS2 (SlMKS2, Genbank Accession No. ACG69783.1 GI:196122243) is derived from S. lycopersicum (cultivated var. M82, Deposit Voucher Accession No. LA1777). The present technology further includes aspects relating to the isolation and characterization of genes, proteins, enzymes involved in fatty acid biosynthesis named methylketone synthase 2 (MKS2).

In some embodiments, the present technology provides a Methylketone Synthase 2 (MKS2) protein. In some embodiments, MKS2 proteins (e.g., SlMKS2 and ShMKS2) of the present technology comprise the amino acid sequences provided in SEQ ID NOs: 3 or 4 respectively. As used herein, the terms “MKS2 polypeptide,” “MSK2 peptide,” and “MKS2 protein” are synonymous. In some embodiments, the MKS2 polypeptide amino acid sequence has about 141 amino acids. In some embodiments, the polypeptide is a MKS2 enzyme having methylketone synthase 2 activity.

In some embodiments, the present technology provides methylketone synthase 2 characterized in that it converts a Cₙ₊₁ 3-keto-acid intermediate to an alkyl methylketone varying in carbon length from C₇ to C₂₀. In some embodiments, the present technology provides a polypeptide having hydrolyzing and decarboxylating activity, the activity being characterized by converting β-ketoacyl-acyl-carrier-proteins; e.g., (3-ketoacyls of fatty acids, 3-ketoacyl-ACPs) and 3-ketoacyl CoA (collectively herein referred to as 3-ketoacyl intermediates) to methylketones of various carbon chain lengths. In some embodiments, the present technology relates to polypeptides having methylketone synthase activity, i.e., where the polypeptide converts 3-ketoacyl intermediates in the fatty acid biosynthetic pathway, including 3-β-ketoacyl-ACP and 3-β-ketoacyl-CoA to one or more alkyl methylketones, for example, 2-tridecanone, 2-undecanone and 2-pentadecanone among other alkyl methylketones.

In some embodiments, the present technology provides a polypeptide which is at least 60%, 70%, 80%, 85%, 90%, 95%, or 100% identical to the amino acid sequence of MKS2; for example such as the sequences set forth in SEQ ID NOs: 3 or 4. According to some embodiments, the MKS2 polypeptide of the present technology originates from tomato species, specifically from Solanum section Lycopersicon and the wild type Solanum habrochaites f glabratum (Deposit Voucher Accession No. PI126449). According to some embodiments, the present technology provides a polypeptide which is at least 60%, 70%, 80%, 85%, 90%, 95%, or 100% homologous (similar+identical amino acids) to MKS2. In some embodiments, the MKS peptide having varying identity or homology has methylketone synthase activity and in some embodiments the MKS peptide having varying identity or homology does not have methylketone synthase activity. For example, polypeptides that do not have methylketone synthase 2 activity are useful in various ways, including use in structural analyses, protein engineering of alternative enzymatic activities, and for generating antibodies to various epitopes, among other uses. Polypeptides having methylketone synthase 2 activity can be used to generate methylketones.

In some embodiments, the present technology provides methods for the production, isolation, and/or purification of the MKS2 polypeptide having methylketone synthase 2 activity, as well as for the products of its enzymatic activity, including methylketones having a C₇-C₂₀ backbone, including, 2-heptanone (C₇ backbone), 2-nonanone (C₉ backbone), 2-tridecanone (2-TD, C₁₃ backbone), 2-undecanone (2-UD, C₁₁ backbone) and 2-pentadecanone (2-PD, C₁₅ backbone).

In some embodiments, the MKS2 polynucleotide of the present technology is originated from Solanum habrochaites S. Knapp & D. M. Spooner and Lycopersicon hirsutum f glabratum (Deposit Voucher Accession No. PI 126449). In some embodiments, the polynucleotide encoding MKS2 (SlMKS2, Genbank Accession No. ACG69783.1 GI:196122243) is derived from S. lycopersicum (cultivated var. M82, Deposit Voucher Accession No. LA1777).

In some embodiments, a gene encoding MKS2 may be incorporated into an organism capable of synthesizing methylketones from 3-ketoacyl intermediates, for example 3-ketoacyl-ACPs and 3-ketoacyl-CoA intermediates, either intracellularly or in cell cultures derived thereof. The MKS2-encoding polynucleotide and necessary expression elements, including promoters, response elements, UTRs, and termination signals may be incorporated into the organism for a variety of purposes, including but not limited to production of MKS2 and production of methylketones, such as those having a carbon backbone ranging from C₇ to C₂₀.

The present technology also provides methods for using MKS2 polypeptides to enzymatically produce products, specifically C₇ through C₂₀ alkyl methylketones. The methylketone products can be used in various processes, including uses in the agricultural, pesticide, chemical, cosmetic, and food industries.

The present technology also provides polynucleotide sequences encoding one or more forms of MKS2 polypeptides, including recombinant DNA molecules, expression vectors, and transformed or transfected host cells or organisms. The present technology further provides nucleic acid vectors and host cells, including vectors comprising the polynucleotides of the present technology, host cells engineered to contain the polynucleotides of the present technology, and host cells engineered to express the polynucleotides of the present technology and synthesize MKS2 and functional variants, derivatives, and homologs thereof. Thus, the present technology provides in some embodiments methods for (i) expressing recombinant MKS2 nucleotides, (e.g., ShMKS2 and SlMKS2), to facilitate the production, isolation and purification of significant quantities of recombinant MKS2, or of its primary and secondary products for subsequent use; (ii) expressing or enhancing the expression of a methylketone synthase, specifically MKS2, in microorganisms, including bacteria, yeast, plants, insects, and animal cells; and (iii) regulating the expression of MKS2, in an environment where such regulation of expression is desired for the production of the enzyme and for producing the enzyme products and derivatives thereof.

The present technology further provides polynucleotide sequences encoding methylketone synthase (e.g., MKS2), for use in a variety of methods and techniques known to those skilled in the art of molecular biology, including, but not limited to the use as hybridization probes, as oligomers for PCR, for chromosome and gene mapping, and the like.

According to some embodiments, the present technology provides an isolated polynucleotide comprising a genomic, complementary, or composite polynucleotide sequence encoding a MKS2 enzyme, the MKS2 capable of converting Cₙ₊₁ 3-keto-acid intermediate to an alkyl methylketone varying in carbon length from C₇ to C₂₀.

According to some embodiments, the present technology provides an isolated polynucleotide comprising a nucleic acid sequence selected from the group consisting of:

(a) the nucleic acid sequence of SEQ ID NOs: 36, 37, 38, or 39;

(b) a nucleic acid sequence encoding the amino acid sequence of SEQ ID NOs: 3 or 4;

(c) the complement of (a) or (b);

(d) a nucleic acid sequence which is at least 60%, 70%, 80%, 85%, 90%, 95%, or 100% identical or homologous to (a), (b), or (c);

(e) a nucleic acid sequence capable of hybridizing under high stringency conditions to (a), (b), or (c); and

(f) an RNA version of (a), (b), (c), or (d).

According to some embodiments, the present technology provides a polynucleotide comprising a nucleic acid sequence encoding a MKS2 comprising the amino acid sequence of SEQ ID NOs: 3 or 4.

According to some embodiments, the present technology provides an isolated polynucleotide comprising a nucleic acid sequence encoding a MKS2 which is at least 60%, 70%, 80%, 85%, 90%, 95%, or 100% identical to the amino acid sequence set forth in SEQ ID NOs: 3 or 4. In some embodiments, the present technology provides an isolated polynucleotide comprising a nucleic acid sequence encoding a MKS2 which is at least 60%, 70%, 80%, 85%, 90%, 95%, or 100% homologous (similar+identical amino acids) to the amino acid sequence set forth in SEQ ID NOs: 3 or 4. The present technology also provides an isolated polynucleotide comprising a nucleic acid sequence which hybridizes under high stringency conditions to a polynucleotide encoding an MKS2 polypeptide, such as those comprising the amino acid sequence of SEQ ID NOs: 3 or 4, including fragments, derivatives, and analogs thereof. The present technology further provides an isolated polynucleotide comprising a nucleic acid sequence which is complementary to the polynucleotide encoding a MKS2 enzyme comprising the amino acid sequence of SEQ ID NOs: 3 or 4 and fragments, derivatives and analogs thereof.

In some embodiments, the present technology provides an isolated polynucleotide comprising a genomic, complementary, or composite polynucleotide sequence encoding MKS2 protein, the MKS2 protein being capable of converting 3-ketoacyl intermediates to methylketones varying in carbon length from C₇ to C₂₀, including for example, 2-tridecanone, 2-undecanone, and/or 2-pentadecanone, among other methylketones.

In some embodiments, the present technology provides a cDNA encoding MKS2 polypeptide. The cDNA or the other polynucleotides encoding MKS polypeptide can be used in many ways, including the development of efficient expression systems for functional enzyme(s), used for examining the developmental regulation of methylketone biosynthesis, used for investigation of the reaction mechanism(s) of the enzyme, and used in the transformation of a wide range of organisms in order to introduce methylketone biosynthesis de novo, or to modify endogenous methylketone biosynthesis.

According to some embodiments, the present technology provides an expression vector comprising a nucleic acid sequence encoding a MKS2 polypeptide.

In some embodiments, the present technology provides an expression vector comprising a nucleic acid sequence selected from the group consisting of:

(a) the nucleic acid sequence of SEQ ID NOs: 36, 37, 38, or 39;

(b) a nucleic acid sequence encoding the amino acid sequence of SEQ ID NOs: 3 or 4;

(c) the complement of (a) or (b);

(d) a nucleic acid sequence which is at least 60%, 70%, 80%, 85%, 90%, 95%, or 100% identical or homologous to (a), (b), or (c);

(e) a nucleic acid sequence capable of hybridizing under high stringency conditions to (a), (b), or (c); and

(f) an RNA version of (a), (b), (c), or (d).

According to other embodiments, the present technology provides an expression vector comprising a polynucleotide sequence encoding MKS2 having an amino acid sequence as provided in SEQ ID NOs: 3 or 4.

In some embodiments, the present technology provides an expression vector comprising a polynucleotide sequence encoding a MKS2 which is at least 60%, 70%, 80%, 85%, 90%, 95%, or 100% identical to the amino acid sequence set forth in SEQ ID NOs: 3 or 4. In some embodiments, the present technology provides an expression vector comprising a polynucleotide sequence encoding a MKS2 which is at least 60%, 70%, 80%, 85%, 90%, 95%, or 100% homologous (similar+identical amino acids) to the amino acid sequence set forth in SEQ ID NOs: 3 or 4.

Various expression systems can be used in the present technology, including prokaryotic and eukaryotic expression systems, for the production of MKS2 polypeptide and its methylketone products, including 2-heptanone (C₇ backbone), 2-nonanone (C₉ backbone), 2-tridecanone (2-TD, C₁₃ backbone), 2-undecanone (2-UD, C₁₁ backbone) and 2-pentadecanone (2-PD, C₁₅ backbone). These expression systems can comprise the necessary elements for posttranslational modification enabling the proper activity of the MKS2 enzyme, as well as the necessary substrates for the synthesis of methylketones, and/or the enzymes for the synthesis of downstream methylketone metabolites.

According to some embodiments, the present technology provides a host cell comprising an expression vector of the present technology. In some embodiments, a method is provided for producing recombinant MKS2, the method comprising: a) culturing the host cell containing the expression vector comprising at least a fragment of the polynucleotide sequence encoding MKS2 under conditions suitable for the expression of the enzyme; and b) recovering the MKS2 enzyme from the host cell culture. According to some embodiments, the present technology provides a method for producing amounts of one or more methylketones having a C₇-C₂₀ backbone, including, 3-ketoacyls of fatty acids (3-ketoacyl-ACPs and 3-ketoacyl-CoA) including, 2-heptanone (C₇ backbone), 2-nonanone (C₉ backbone), 2-tridecanone (2-TD, C₁₃ backbone), 2-undecanone (2-UD, C₁₁ backbone) and 2-pentadecanone (2-PD, C₁₅ backbone), the method comprising: a) culturing the host cell containing an expression vector comprising at least a fragment of the polynucleotide sequence encoding MKS2 under conditions suitable for the expression and activity of the enzyme; and b) recovering one or more methylketones from the host cell culture.

Methylketones produced within a host cell according to the present technology can serve as a substrate for producing additional compounds. For example, chemical and/or enzymatic procedures present in the host cell that have reaction activities downstream to MKS2 in the fatty acid biosynthetic pathway can be used. Such compounds are designated herein as “methylketone metabolites.” For example, MKS2 polypeptide can be expressed along with MKS1 polypeptide in a host cell.

In some embodiments, the present technology provides a method for producing one or more methylketones in a fatty acid biosynthesis pathway, the method comprising: a) culturing the host cell containing an expression vector comprising at least a fragment of the polynucleotide sequence encoding MKS2 under conditions suitable for the expression and activity of the MKS2; and b) recovering the one or more methylketones from the host cell culture. According to some embodiments, the methylketones can include 2-heptanone, 2-nonanone, 2-tridecanone, 2-undecanone, and 2-pentadecanone, among others.

In some embodiments, the present technology provides a prokaryotic organism or a eukaryotic organism comprising a polynucleotide sequence encoding a MKS2, for example, ShMKS2 (SEQ ID NO: 4) and/or SlMKS2 (SEQ ID NO: 3) stably integrated into its genome. In some embodiments, the host cell can comprise a plant cell, a whole plant, or a substructure of a plant. For example, the host cell may comprise a plant root cell, plant leaf cell, or plant flower or seed cell. In some embodiments, the host cell may comprise a prokaryotic cell that is associated with a plant cell. For example, the host cell may comprise a diazotroph (e.g., Rhizobia), Agrobacterium tumefaciens, or other such cell known in the art.

According to some embodiments, the present technology provides a prokaryotic organism comprising a nucleic acid sequence encoding the polypeptide of SEQ ID NOs: 3 or 4, the complement of a nucleic acid sequence encoding the polypeptide of SEQ ID NOs: 3 or 4, a nucleic acid sequence which is at least 85%, 90%, or 95% identical or homologous to a nucleic acid sequence encoding the polypeptide of SEQ ID NOs: 3 or 4, a nucleic acid sequence capable of hybridizing to a nucleic acid sequence encoding the polypeptide of SEQ ID NOs: 3 or 4, and a nucleic acid sequence capable of hybridizing to the complement of a nucleic acid sequence encoding the polypeptide of SEQ ID NOs: 3 or 4 stably integrated into its genome. According to some embodiments, the present technology provides a prokaryotic organism comprising a polynucleotide sequence encoding a MKS2 polypeptide stably integrated into its genome.

According to yet further embodiments, the present technology provides a prokaryotic organism comprising a polynucleotide encoding MKS2 polypeptide stably integrated into its genome, the prokaryotic organism producing a methylketone having a carbon C₇ to C₂₀ backbone. In some embodiments, the prokaryotic organism is E. coli.

According to yet another aspect, the present technology provides one or more methylketones having a carbon C₇ to C₂₀ backbone obtained by the methods of the present technology for industrial uses. In some embodiments, the present technology provides a method for producing methylketones, the method comprising: a) culturing the host cell containing an expression vector comprising at least a fragment of the polynucleotide sequence encoding MKS2 under conditions suitable for the expression and activity of the MKS2; and b) recovering said alkyl methylketones from the host cell culture. The methylketones can have a carbon backbone ranging from C₇ to C₂₀. According to yet another aspect, the present technology provides a prokaryotic or eukaryotic organism in which significant amounts of methylketones are synthesized. According to some embodiments, the present technology provides a prokaryotic organism comprising a polynucleotide sequence encoding a MKS2 stably integrated into its genome.

As is known to a person skilled in the art, many bacterial strains are suitable as host cells for the over-expression of MKS proteins according to the present technology, including E. coli strains and many other species and genera of prokaryotes including bacilli such as Bacillus subtilis, other enterobacteriaceae such as Salmonella typhimurium or Serratia marcesans, and various Pseudomonas species. Prokaryotic host cells or other host cells with rigid cell walls can be transformed using a calcium chloride method as described in section 1.82 of Sambrook et al., Molecular Cloning—A Laboratory Manual (3rd Ed.), Vol. 1-3, Cold Spring Harbor Laboratory, Cold Spring Harbor, N.Y., 2000. Alternatively, electroporation may be used for transformation of such cells. Various prokaryote transformation techniques are known in the art; e.g. Dower, W. J., in Genetic Engineering, Principles and Methods, 12:275-296, Plenum Publishing Corp., 1990; Hanahan et al., Meth. Enzymol., 204:63 1991.

In some embodiments, the present technology provides methylketones, including for example, 2-heptanone (C₇ backbone), 2-nonanone (C₉ backbone), 2-tridecanone (2-TD, C₁₃ backbone), 2-undecanone (2-UD, C₁₁ backbone) and 2-pentadecanone (2-PD, C₁₅ backbone). These methylketones can be obtained using the methods of the present technology and can be used in various industrial processes and for making various products, including methods and products relating to agricultural, pesticide, cosmetic, and food products.

According to yet another aspect, the present technology provides one or more alky methylketones (having a C₇ to C₂₀ carbon backbone, for example, 2-heptanone (C₇ backbone), 2-nonanone (C₉ backbone), 2-tridecanone (2-TD, C₁₃ backbone), 2-undecanone (2-UD, C₁₁ backbone) and 2-pentadecanone (2-PD, C₁₅ backbone) from a 3-ketoacyl intermediates, in the fatty acid biosynthesis (or fatty acid degradation) pathway (in the form of 3-ketoacyl-ACP or 3-ketoacyl-CoA respectively). These alkyl methylketones thus obtained by the methods of the present technology are valuable feedstocks for industrial uses, for example, for use in pesticide, cosmetic and other chemical manufacturing processes. According to one embodiment, the present technology provides 2-heptanone, 2-nonanone, 2-tridecanone, 2-undecanone and 2-pentadecanone in addition to other alky methylketones obtained for use as a feed stock chemical or used “as is” in the synthesis of a product selected from the agricultural, pesticide, chemical, cosmetic, pharmaceutical and food products.

Sequence Comparison, Identity, and Homology

The terms “identical” or “percent identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill) or by visual inspection.

The phrase “substantially identical,” in the context of two nucleic acids or polypeptides (e.g., DNAs encoding a fatty acid synthase (FAS), polyketide synthase (PKS), fusion protein, or domain thereof, or the amino acid sequence of a FAS, PKS, fusion protein, or domain thereof) refers to two or more sequences or subsequences that have at least about 60%, about 80%, about 85%, about 90-95%, about 98%, about 99% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, the “substantial identity” exists over a region of the sequences that is at least about 50 residues in length, more preferably over a region of at least about 100 residues, and most preferably, the sequences are substantially identical over at least about 150 residues, or over the full length of the two sequences to be compared.

Polypeptides and proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity (e.g., identity) over 50, 100, 150 or more residues (nucleotides or amino acids) is routinely used to establish homology (e.g., over the full length of the two sequences to be compared). Higher levels of sequence similarity (e.g., identity), e.g., 30%, 40%, 50%, 60%, 70%, 80%, 85%, 90%, 95%, or 99% or more, can also be used to establish homology. Methods for determining sequence similarity percentages (e.g., BLASTP and BLASTN using default parameters) are described herein and are generally available.

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

MKS2 Variants, Homologs and Derivatives

The terms “alteration”, “amino acid sequence alteration”, “variant” and “amino acid sequence variant” refer to MKS2 molecules with some differences in their amino acid sequences as compared to MKS2 from specific plants, including, Solanum section Lycopersicon and Arabidopsis, especially MKS2 of the wild type tomato Solanum habrochaites F glabratum (ShMKS2) as provided by the amino acid sequence set forth in SEQ ID NO: 4 and (SlMKS2) derived from the cultivated tomato plant S. lycopersicum (var. M82) having the amino acid sequence provided in SEQ ID NO: 3. Ordinarily, the variants can possess at least about 70% homology, preferably at least about 80%, most preferably at least about 85% or at least 90% homology with the above defined MKS2 polypeptides provided in SEQ ID NOs: 3 or 4. The amino acid sequence variants of MKS2 falling within this technology possess substitutions, deletions, and/or insertions at certain positions. Sequence variants of MKS2 may be used to attain desired enhanced enzymatic activity or altered substrate utilization or product distribution. Substitutional MKS2 variants are those that have at least one amino acid residue in the MKS2 sequence set forth in SEQ ID NOs: 3 or 4 removed and a different amino acid inserted in its place at the same position. The substitutions may be single, where only one amino acid in the molecule has been substituted, or they may be multiple, where two or more amino acids have been substituted in the same molecule. Substantial changes in the activity of the MKS2 molecules of the present technology may be obtained by substituting an amino acid with a side chain that is significantly different in charge and/or structure from that of the native amino acid. This type of substitution would be expected to affect the structure of the polypeptide backbone and/or the charge or hydrophobicity of the molecule in the area of the substitution.

Moderate changes in the activity of the MKS2 molecules of the present technology would be expected by substituting an amino acid with a side chain that is similar in charge and/or structure to that of the native molecule. This type of substitution, referred to as a conservative substitution, would not be expected to substantially alter either the structure of the polypeptide backbone or the charge or hydrophobicity of the molecule in the area of the substitution.

Insertional MKS2 variants are those with one or more amino acids inserted immediately adjacent to an amino acid at a particular position in the amino acid sequence of MKS2 set forth in SEQ ID NOs: 3 or 4. Immediately adjacent to an amino acid means connected to either the α-carboxy or α-amino functional group of the amino acid. The insertion may be one or more amino acids. Ordinarily, the insertion will consist of one or two conservative amino acids. Amino acids similar in charge and/or structure to the amino acids adjacent to the site of insertion are defined as conservative. Alternatively, this technology includes insertion of an amino acid with a charge and/or structure that is substantially different from the amino acids adjacent to the site of insertion.

Deletional variants are those where one or more amino acids in the amino acid sequence of MKS2 set forth in SEQ ID NOs: 3 or 4 have been removed. Ordinarily, deletional variants will have one or two amino acids deleted in a particular region of the MKS2 molecule.

The term “biological activity”, “biologically active”, “activity” and “active” refer to the ability of the MKS2 to convert 3-ketoacyl intermediates (e.g. 3-β-ketoacyl-ACP and 3-β-ketoacyl-CoA) to one or more methylketones, for example, 2-tridecanone, 2-undecanone and 2-pentadecanone. The MKS2 can be expressed in a variety of host cells, including, plants, animals, yeasts, and bacteria.

The terms “DNA sequence encoding”, “DNA encoding”, “nucleic acid encoding” or “polynucleotide sequence encoding” refer to the order or sequence of deoxyribonucleotides along a strand of deoxyribonucleic acid. The order of these deoxyribonucleotides determines the order of amino acids along the translated polypeptide chain. The DNA sequence thus codes for the amino acid sequence.

The term “hybridization”, as used herein, refers to any process by which a strand of nucleic acid binds with a complementary strand through base pairing.

The terms “stringent conditions” or “stringency”, as used herein, refer to the conditions for hybridization as defined by the nucleic acid, salt, and temperature. These conditions are well known in the art and may be altered in order to identify or detect identical or related polynucleotide sequences. Numerous equivalent conditions comprising either low or high stringency depend on factors such as the length and nature of the sequence (DNA, RNA, base composition), nature of the target (DNA, RNA, base composition), milieu (in solution or immobilized on a solid substrate), concentration of salts and other components (e.g., formamide, dextran sulfate and/or polyethylene glycol), and temperature of the reactions (within a range from about 5° C. to about 25° C. below the melting temperature of the probe). One or more factors may be varied to generate conditions of either low or high stringency.

In some embodiments, a “replicable expression vector” and “expression vector” can refer to a piece of DNA, usually double-stranded, which may have inserted into it a piece of foreign DNA. Foreign DNA is defined as heterologous DNA, which is DNA not naturally found in the host. The vector is used to transport the foreign or heterologous DNA into a suitable host cell. Once in the host cell, the vector can replicate independently of or coincidental with the host chromosomal DNA, and several copies of the vector and its inserted (foreign) DNA may be generated. In addition, the vector contains the necessary elements that permit translating the foreign DNA into a polypeptide. Many molecules of the polypeptide encoded by the foreign DNA can thus be rapidly synthesized.

The terms “transformed host cell”, “transformed” and “transformation” refer to the introduction of DNA into a cell. The cell is termed a “host cell”, and it may be a prokaryotic or a eukaryotic cell. Typical prokaryotic host cells include various strains of E. coli. and can also include other bacterial strains capable of expressing a partial or full length MKS2 polypeptide. Typical eukaryotic host cells are plant cells, yeast cells, insect cells or animal cells. The introduced DNA is usually in the form of a vector containing an inserted piece of DNA. The introduced DNA sequence may be from the same species as the host cell or from a different species from the host cell, or it may be a hybrid DNA sequence, containing some foreign DNA and some DNA derived from the host species.

Conservative Variations

Owing to the degeneracy of the genetic code, “silent substitutions” (i.e., substitutions in a nucleic acid sequence which do not result in an alteration in an encoded polypeptide) are an implied feature of every nucleic acid sequence that encodes an amino acid sequence. Similarly, “conservative amino acid substitutions,” where one or a limited number of amino acids in an amino acid sequence are substituted with different amino acids with highly similar properties, are also readily identified as being highly similar to a disclosed construct. Such conservative variations of each disclosed sequence are a feature of the present invention.

“Conservative variations” of a particular polynucleotide sequence refers to those nucleic acids which encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid, while retaining the relevant function of the polypeptide such as enzymatic activity (for example, the conservative substitution can be of a residue distal to the active site region). Thus, “conservative variations” of a listed polypeptide sequence of the present invention include substitutions of a small percentage, typically less than 5%, more typically less than 2% or 1%, of the amino acids of the polypeptide sequence, with an amino acid of the same conservative substitution group. Finally, the addition of sequences which do not alter the encoded activity of a nucleic acid molecule, such as the addition of a non-functional or tagging sequence (introns in the nucleic acid, poly His or similar sequences in the encoded polypeptide, etc.), is a conservative variation of the basic nucleic acid or polypeptide.

Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the polypeptide molecule. The following sets forth example groups that contain natural amino acids of like chemical properties, where substitution within a group is a “conservative substitution.” It will be evident that a variety of similar tables exist in the art, and that conservative vs. non-conservative substitutions can be classified, e.g., based on steric bulk and/or hydropathy (e.g., taking into account the Kyte/Doolittle hydropathy index and/or structural statistics comparing trends (solvent-exposed or buried) observed in proteins for each residue.

TABLE II
Conservative amino acid substitutions known in the art
Conservative Amino Acid Substitutions

| Nonpolar | Polar, | | Positively | Negatively |
|---|---|---|---|---|
| and/or | Uncharged | Aromatic | charged | charged |
| Aliphatic Side | side | side | side | side |
| Chains | chains | chains | chains | chains |
| Glycine | Serine | Phenylalanine | Lysine | Aspartate |
| Alanine | Threonine | Tyrosine | Arginine | Glutamate |
| Valine | Cysteine | Tryptophan | Histidine | |
| Leucine | Methionine | | | |
| Isoleucine | Asparagine | | | |
| Proline | Glutamine | | | |

Nucleic Acid Hybridization

Comparative hybridization can be used to identify nucleic acids of the present technology, including conservative variations of nucleic acids of the invention. In addition, target nucleic acids which hybridize to a nucleic acid of the present technology under high, ultra-high and ultra-ultra high stringency conditions, where the nucleic acids are other than a naturally occurring nucleic acid, are a feature of the present technology. Examples of such nucleic acids include those with one or a few silent or conservative nucleic acid substitutions as compared to a given nucleic acid sequence of the invention.

A test nucleic acid is said to specifically hybridize to a probe nucleic acid when it hybridizes at least 50% as well to the probe as to the perfectly matched complementary target, i.e., with a signal to noise ratio at least half as high as hybridization of the probe to the target under conditions in which the perfectly matched probe binds to the perfectly matched complementary target with a signal to noise ratio that is at least about 5×-10× as high as that observed for hybridization to any of the unmatched target nucleic acids.

Nucleic acids “hybridize” when they associate, typically in solution. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology—Hybridization with Nucleic Acid Probes part I chapter 2, “Overview of principles of hybridization and the strategy of nucleic acid probe assays,” (Elsevier, N.Y.), as well as in Ausubel; Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) provide details on the synthesis, labeling, detection and quantification of DNA and RNA, including oligonucleotides.

An example of stringent hybridization conditions for hybridization of complementary nucleic acids which have more than 100 complementary residues on a filter in a Southern or northern blot is 50% formalin with 1 mg of heparin at 42° C., with the hybridization being carried out overnight. An example of stringent wash conditions is a 0.2×SSC wash at 65° C. for 15 minutes (see Sambrook et al., Molecular Cloning—A Laboratory Manual (3rd Ed.), Vol. 1-3, Cold Spring Harbor Laboratory, Cold Spring Harbor, N.Y., 2000 for a description of SSC buffer). Often the high stringency wash is preceded by a low stringency wash to remove background probe signal. An example low stringency wash is 2×SSC at 40° C. for 15 minutes. In general, a signal to noise ratio of 5× (or higher) than that observed for an unrelated probe in the particular hybridization assay indicates detection of a specific hybridization.

Stringent hybridization wash conditions in the context of nucleic acid hybridization experiments such as Southern and northern hybridizations are sequence dependent, and are different under different environmental parameters. An extensive guide to the hybridization of nucleic acids is found in Tijssen (1993), supra and in Hames and Higgins (1995) Gene Probes 1 IRL Press at Oxford University Press, Oxford, England, (Hames and Higgins 1) and Hames and Higgins (1995) Gene Probes 2 IRL Press at Oxford University Press, Oxford, England (Hames and Higgins 2) wherein the subject matter relating to nucleic acid hybridization being incorporated herein in its entirety. Stringent hybridization and wash conditions can easily be determined empirically for any test nucleic acid. For example, in determining stringent hybridization and wash conditions, the hybridization and wash conditions are gradually increased (e.g., by increasing temperature, decreasing salt concentration, increasing detergent concentration and/or increasing the concentration of organic solvents such as formalin in the hybridization or wash), until a selected set of criteria are met. For example, in highly stringent hybridization and wash conditions, the hybridization and wash conditions are gradually increased until a probe binds to a perfectly matched complementary target with a signal to noise ratio that is at least 5× as high as that observed for hybridization of the probe to an unmatched target.

“Very stringent” conditions are selected to be equal to the thermal melting point (Tₘ) for a particular probe. The Tₘ is the temperature (under defined ionic strength and pH) at which 50% of the test sequence hybridizes to a perfectly matched probe. For the purposes of the present technology, generally, “highly stringent” or “high stringency” hybridization and wash conditions are selected to be about 5° C. lower than the Tₘ for the specific sequence at a defined ionic strength and pH.

“Ultra high-stringency” hybridization and wash conditions are those in which the stringency of hybridization and wash conditions are increased until the signal to noise ratio for binding of the probe to the perfectly matched complementary target nucleic acid is at least 10× as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-high stringency conditions.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10×, 20×, 50×, 100×, or 500× or more as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-ultra-high stringency conditions.

Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code.

Creation of MKS2 Sequence Mutants, Derivatives and Homologs

In addition to the native MKS2 amino acid sequence, sequence variants produced by deletions, substitutions, mutations and/or insertions are intended to be within the scope of the present technology. The MKS2 amino acid sequence variants of the present technology can be constructed by mutating the DNA sequence that encodes the wild-type MKS2 comprising the amino acid sequence of SEQ ID NOs: 3 or 4, such as by using techniques commonly referred to as site-directed mutagenesis. Nucleic acid molecules encoding the MKS2 of the present technology can be mutated by a variety of PCR techniques well known to one of ordinary skill in the art. See, e.g., ‘PCR Strategies,” M. A. Innis, D. H. Gelfand and J. J. Sninsky, eds., 1995, Academic Press, San Diego, Calif. (Chapter 14); “PCR Protocols: A Guide to Methods and Applications,” M. A. Innis, D. H. Gelfand, J. J. Sninsky and T. J. White, eds., Academic Press, NY (1990).

By way of non-limiting example, the two-primer system utilized in the Transformer Site-Directed Mutagenesis kit from Clontech, may be employed for introducing site-directed mutants into the MKS2 gene of the present technology. Following denaturation of the target plasmid in this system, two primers are simultaneously annealed to the plasmid; one of these primers contains the desired site-directed mutation, the other contains a mutation at another point in the plasmid resulting in elimination of a restriction site. Second strand synthesis is then carried out, tightly linking these two mutations, and the resulting plasmids are transformed into a mutS strain of E. coli. Plasmid DNA is isolated from the transformed bacteria, restricted with the relevant restriction enzyme (thereby linearizing the unmutated plasmids), and then retransformed into E. coli. This system allows for generation of mutations directly in an expression plasmid, without the necessity of subjoining or generation of single-stranded phagemids. The tight linkage of the two mutations and the subsequent linearization of unmutated plasmids result in high mutation efficiency and allow minimal screening. Following synthesis of the initial restriction site primer, this method requires the use of only one new primer type per mutation site. Rather than prepare each positional mutant separately, a set of “designed degenerate” oligonucleotide primers can be synthesized in order to introduce all of the desired mutations at a given site simultaneously. Transformants can be screened by sequencing the plasmid DNA through the mutagenized region to identify and sort mutant clones. Each mutant DNA can then be restricted and analyzed to confirm that no other alterations in the sequence have occurred (e.g., by band shift comparison to the unmutagenized control).

In the design of a particular site directed mutagenesis, it is generally desirable to first make a non-conservative substitution (e.g., Ala for Cys, His or Glu) and determining if activity is greatly impaired as a consequence. The properties of the mutagenized protein are then examined with particular attention to the kinetic parameters of Kₘ and k_{cat} as sensitive indicators of altered function, from which changes in binding and/or catalysis per site may be deduced by comparison to the native enzyme. If the residue is demonstrated to be important by activity impairment, or knockout, then conservative substitutions can be made, such as Asp for Glu to alter side chain length, Ser for Cys, or Arg for His. For hydrophobic segments, it is commonly size that is usefully altered, although aromatics can also be substituted for alkyl side chains. Changes in the normal product distribution can indicate which step(s) of the reaction sequence have been altered by the mutation. Modification of the hydrophobic pocket can be employed to change binding conformations for substrates.

Other site directed mutagenesis techniques might also be employed with the nucleotide sequences of the technology. For example, restriction endonuclease digestion of DNA followed by ligation may be used to generate deletion variants of MKS2, as described in section 15.3 of Sambrook et al. (Molecular Cloning: A Laboratory Manual, 2nd Ed., Cold Spring Harbor Laboratory Press, New York, N.Y. 1989). A similar strategy may be used to construct insertion variants, as described in section 15.3 of Sambrook et al., supra.

Oligonucleotide-directed mutagenesis may also be employed for preparing substitution variants of this technology. It may also be used to conveniently prepare the deletion and insertion variants of this technology. This technique is well known in the art as described, for example, by Adelman et al. (DNA 2:183 1983); Sambrook et al., supra; “Current Protocols in Molecular Biology”, 1991, Wiley (NY), F. T. Ausubel, R. Brent, R. E. Kingston, D. D. Moore, J. D. Seidman, J. A. Smith and K. Struhl, eds.

Generally, oligonucleotides of at least 25 nucleotides in length are used to insert, delete or substitute two or more nucleotides in the nucleic acid molecules encoding MKS2 of the technology. An optimal oligonucleotide will have 12 to 15 perfectly matched nucleotides on either side of the nucleotides coding for the mutation. To mutagenize nucleic acids encoding the native MKS2 of the present technology, the oligonucleotide can be annealed to the single-stranded DNA template molecule under suitable hybridization conditions. A DNA polymerizing enzyme, usually the Klenow fragment of E. coli DNA polymerase I, is then added. This enzyme uses the oligonucleotide as a primer to complete the synthesis of the mutation-bearing strand of DNA. Thus, a heteroduplex molecule is formed such that one strand of DNA encodes the native synthase inserted in the vector, and the second strand of DNA encodes the mutated form of the synthase inserted into the same vector. This heteroduplex molecule is then transformed into a suitable host cell.

Mutants substituted with more than one amino acid may be generated in one of several ways. If the amino acids are located close together in the polypeptide chain, they may be mutated simultaneously using one oligonucleotide that codes for all of the desired amino acid substitutions. If however, the amino acids are located in some distance from each other (e.g., separated by more than ten amino acids) it is more difficult to generate a single oligonucleotide that encodes all of the desired changes. Instead, one of two alternative methods may be employed. In the first method, a separate oligonucleotide is generated for each substituted amino acid. The oligonucleotides are ten annealed to the single-stranded template DNA simultaneously, and the second DNA strand synthesized from the template will encode all of the desired amino acid substitutions. An alternative method involves two or more rounds of mutagenesis to produce the desired mutant. The first round is as described for the single mutants: native MKS2 DNA is used for the template, an oligonucleotide encoding the first desired amino acid substitution is annealed to this template, and the heteroduplex DNA molecule is then generated. The second round of mutagenesis utilizes the mutated DNA produced in the first round of mutagenesis as the template. Thus, this template already contains one or more mutations. The oligonucleotide encoding the additional desired amino acid substitution(s) is then annealed to this template, and the resulting strand of DNA now encodes mutations from both the first and the second rounds of mutagenesis. The mutagenized DNA can then be used as a template in a third round of mutagenesis, and so on.

Other types of mutagenesis can be optionally employed in the present technology, e.g., to introduce convenient restriction sites or to modify specificities of various catalytic domains of MKS2, for example, the “hotdog-fold” domain shared with 4-hydroxybenzoyl-coenzyme A thioesterase (4HBT). It has been found, that specific conservation of Asp17 in MKS2 polypeptides: ShMKS2 and SlMKS2, purports to be directed to 4HBT catalytic activity. In general, any available mutagenesis procedure can be used for making such mutants. Such mutagenesis procedures optionally include selection of mutant nucleic acids and polypeptides for one or more activity of interest (e.g., altered starter or extender unit or product specificity). Procedures that can be used include, but are not limited to: site-directed point mutagenesis, random point mutagenesis, in vitro or in vivo homologous recombination (DNA shuffling), mutagenesis using uracil containing templates, oligonucleotide-directed mutagenesis, phosphorothioate-modified DNA mutagenesis, mutagenesis using gapped duplex DNA, point mismatch repair, mutagenesis using repair-deficient host strains, restriction-selection and restriction-purification, deletion mutagenesis, mutagenesis by total gene synthesis, degenerate PCR, double-strand break repair, and many others known to persons of skill.

Optionally, mutagenesis can be guided by known information from a naturally occurring fatty acid or methylketone synthase or a domain thereof, or of a known altered or mutated methylketone synthase, e.g., sequence, sequence comparisons, physical properties, crystal structure and/or the like as discussed above. However, in some embodiments, modification can be essentially random (e.g., as in classical DNA shuffling). Additional information on mutation formats can be found in Sambrook, Ausubel, and Innis as referenced herein.

The following publications and references provide still additional detail on mutation formats: Arnold, Protein engineering for unusual environments, Current Opinion in Biotechnology 4:450-455 (1993); Bass et al., Mutant Trp repressors with new DNA-binding specificities, Science 242:240-245 (1988); Botstein & Shortle, Strategies and applications of in vitro mutagenesis, Science 229:1193-1201 (1985); Carter et al., Improved oligonucleotide site-directed mutagenesis using M13 vectors, Nucl. Acids Res. 13: 4431-4443 (1985); Carter, Site-directed mutagenesis, Biochem. J. 237:1-7 (1986); Carter, Improved oligonucleotide-directed mutagenesis using M13 vectors, Methods in Enzymol. 154: 382-403 (1987); Dale et al., Oligonucleotide-directed random mutagenesis using the phosphorothioate method, Methods Mol. Biol. 57:369-374 (1996); Eghtedarzadeh & Henikoff, Use of oligonucleotides to generate large deletions, Nucl. Acids Res. 14: 5115 (1986); Fritz et al., Oligonucleotide-directed construction of mutations: a gapped duplex DNA procedure without enzymatic reactions in vitro, Nucl. Acids Res. 16: 6987-6999 (1988); Grundstrom et al., Oligonucleotide-directed mutagenesis by microscale ‘shot-gun’ gene synthesis, Nucl. Acids Res. 13: 3305-3316 (1985); Kunkel, The efficiency of oligonucleotide directed mutagenesis, in Nucleic Acids & Molecular Biology (Eckstein, F. and Lilley, D. M. J. eds., Springer Verlag, Berlin)) (1987); Kunkel, Rapid and efficient site-specific mutagenesis without phenotypic selection, Proc. Natl. Acad. Sci. USA 82:488-492 (1985); Kunkel et al., Rapid and efficient site-specific mutagenesis without phenotypic selection, Methods in Enzymol. 154, 367-382 (1987); Kramer et al., The gapped duplex DNA approach to oligonucleotide-directed mutation construction, Nucl. Acids Res. 12:9441-9456 (1984); Kramer & Fritz Oligonucleotide-directed construction of mutations via gapped duplex DNA, Methods in Enzymol. 154:350-367 (1987); Kramer et al., Point Mismatch Repair, Cell 38:879-887 (1984); Kramer et al., Improved enzymatic in vitro reactions in the gapped duplex DNA approach to oligonucleotide-directed construction of mutations, Nucl. Acids Res. 16: 7207 (1988); Ling et al., Approaches to DNA mutagenesis: an overview, Anal Biochem. 254(2): 157-178 (1997); Lorimer and Pastan Nucleic Acids Res. 23, 3067-8 (1995); Mandecki, Oligonucleotide-directed double-strand break repair in plasmids of Escherichia coli: a method for site-specific mutagenesis, Proc. Natl. Acad. Sci. USA, 83:7177-7181 (1986); Nakamaye & Eckstein, Inhibition of restriction endonuclease Nci I cleavage by phosphorothioate groups and its application to oligonucleotide-directed mutagenesis, Nucl. Acids Res. 14: 9679-9698 (1986); Nambiar et al., Total synthesis and cloning of a gene coding for the ribonuclease S protein, Science 223: 1299-1301 (1984); Sakamar and Khorana, Total synthesis and expression of a gene for the a-subunit of bovine rod outer segment guanine nucleotide-binding protein (transducin), Nucl. Acids Res. 14: 6361-6372 (1988); Sayers et al., Y-T Exonucleases in phosphorothioate-based oligonucleotide-directed mutagenesis, Nucl. Acids Res. 16:791-802 (1988); Sayers et al., Strand specific cleavage of phosphorothioate-containing DNA by reaction with restriction endonucleases in the presence of ethidium bromide, (1988) Nucl. Acids Res. 16: 803-814; Sieber, et al., Nature Biotechnology, 19:456-460 (2001); Smith, In vitro mutagenesis, Ann. Rev. Genet. 19:423-462 (1985); Methods in Enzymol. 100: 468-500 (1983); Methods in Enzymol. 154: 329-350 (1987); Stemmer, Nature 370, 389-91 (1994); Taylor et al., The use of phosphorothioate-modified DNA in restriction enzyme reactions to prepare nicked DNA, Nucl. Acids Res. 13: 8749-8764 (1985); Taylor et al., The rapid generation of oligonucleotide-directed mutations at high frequency using phosphorothioate-modified DNA, Nucl. Acids Res. 13: 8765-8787 (1985); Wells et al., Importance of hydrogen-bond formation in stabilizing the transition state of subtilisin, Phil. Trans. R. Soc. Lond. A 317: 415-423 (1986); Wells et al., Cassette mutagenesis: an efficient method for generation of multiple mutations at defined sites, Gene 34:315-323 (1985); Zoller & Smith, Oligonucleotide-directed mutagenesis using M13-derived vectors: an efficient and general procedure for the production of point mutations in any DNA fragment, Nucleic Acids Res. 10:6487-6500 (1982); Zoller & Smith, Oligonucleotide-directed mutagenesis of DNA fragments cloned into M13 vectors, Methods in Enzymol. 100:468-500 (1983); and Zoller & Smith, Oligonucleotide-directed mutagenesis: a simple method using two oligonucleotide primers and a single-stranded DNA template, Methods in Enzymol. 154:329-350 (1987). Additional details on many of the above methods can be found in Methods in Enzymology Volume 154, which also describes useful controls for trouble-shooting problems with various mutagenesis methods.

An alternative to these mutational methods involves recombining entire genomes of organisms and selecting resulting progeny for particular pathway functions (often referred to as “whole genome shuffling”). This approach can be applied to the present invention, e.g., by genomic recombination and selection of an organism (e.g., an E. coli or other cell) for an ability to produce a desired precursor or product (or intermediate thereof). For example, methods taught in the following publications can be applied to pathway design for the evolution of existing and/or new pathways in cells to produce precursors or products in vivo: Patnaik et al. (2002) “Genome shuffling of lactobacillus for improved acid tolerance” Nature Biotechnology 20(7):707-712; and Zhang et al. (2002) “Genome shuffling leads to rapid phenotypic improvement in bacteria” Nature 415:644-646.

Other techniques for organism and metabolic pathway engineering, e.g., for the production of desired compounds, are also available and can also be applied to the production of precursors or products. Examples of publications teaching useful pathway engineering approaches include: Nakamura and White (2003) “Metabolic engineering for the microbial production of 1,3 propanediol” Curr. Opin. Biotechnol. 14(5):454-9; Berry et al. (2002) “Application of Metabolic Engineering to improve both the production and use of Biotech Indigo” J. Industrial Microbiology and Biotechnology 28:127-133; Banta et al. (2002) “Optimizing an artificial metabolic pathway: Engineering the cofactor specificity of Corynebacterium 2,5-diketo-D-gluconic acid reductase for use in vitamin C biosynthesis” Biochemistry 41(20):6226-36; Selivonova et al. (2001) “Rapid Evolution of Novel Traits in Microorganisms” Applied and Environmental Microbiology 67:3645, and many others.

Regardless of the method used, typically, the precursor(s) produced with an engineered biosynthetic pathway of the invention can be produced in a concentration sufficient for efficient MKS2 substrate (3-ketoacyl-ACPs and/or 3-ketoacyl-CoA intermediates) utilization for methylketone biosynthesis and/or fatty acid degradation. The precursors thus produced in the host cell can include, a natural cellular amount, but not to such a degree as to significantly affect the concentration of other cellular compounds or to exhaust cellular resources. Once a host cell is engineered to produce one or more MKS2 enzymes desired for a specific pathway and a precursor is generated, in vivo selections are optionally used to further optimize the production of the precursor for methylketone synthesis.

A variety of kits for performing mutagenesis are commercially available (see, e.g., the QuikChange® site-directed mutagenesis kit from Stratagene and the BD Transformer™ site-directed mutagenesis kit from Clontech).

Heterologous Expression Systems

In one aspect, the present technology provides a host cell in which a recombinant protein (e.g., a recombinant MKS2 protein) of the present technology is heterologously expressed from a polynucleotide sequence capable of encoding MKS2 comprising an amino acid sequence of SEQ ID NO: 3 or 4. In some embodiments, the present technology provides a host cell containing an expression vector of the present technology. The present technology further provides a method for the production of recombinant MKS2, the method comprising a) culturing a host cell containing an expression vector comprising at least a fragment of the polynucleotide sequence encoding MKS2, (for example a polynucleotide encoding SEQ ID NO: 3 or 4) under conditions suitable for the expression of the MKS2; and b) recovering MKS2 from the host cell culture.

The host cell may be transformed with the expression vector according to the present technology by using any known method for the insertion of foreign nucleic acid sequences into a prokaryotic or eukaryotic host cell. The transformation process results in the expression of the inserted DNA such as to change the recipient cell into a transformed, genetically modified or transgenic cell.

In some embodiments, the present technology provides a host cell comprising an expression vector that includes a functional promoter operably linked to a polynucleotide encoding a MKS2 protein, which MKS2 protein comprises at least one MKS2 polypeptide or hydrolyzing and/or decarboxylation catalytic domain thereof. In addition, a plethora of kits are commercially available for the purification of plasmids or other relevant nucleic acids from cells, (see e.g., EasyPrep™, FlexiPrep™, both from Pharmacia Biotech; StrataClean™, from Stratagene; and, QIAprep™ from Qiagen). Any isolated and/or purified nucleic acid can be further manipulated to produce other nucleic acids, used to transfect cells, incorporated into related vectors to infect organisms for expression, and/or the like.

Vectors of various types may be used in the practice of the present technology. A specific vector type is used according to the host cell in which expression is desired, as is known to a person with ordinary skill in the art, and as described herein below. The vector usually has a replication site, marker genes that provide phenotypic selection in transformed cells, one or more functional promoters, and a polylinker region containing several restriction sites for insertion of foreign DNA. For example, plasmids typically used for transformation of E. coli include pBR322, pUC 18, pUC 19, pUCI18, pUC1 19, and Bluescript M13, all of which are described in sections 1.12-1.20 of Sambrook et al., supra. These vectors contain genes coding for ampicillin and/or tetracycline resistance, which enables cells transformed with these vectors to grow in the presence of these antibiotics. However, many other suitable vectors, harboring different genes encoding for selection markers are available as well. The construction of suitable vectors containing DNA encoding replication sequences, regulatory sequences, phenotypic selection genes and the MKS2 DNA of interest are prepared using standard recombinant DNA procedures. Isolated plasmids and DNA fragments are cleaved, tailored, and ligated together in a specific order to generate the desired vectors, as is well known in the art (see, for example, Sambrook et al., supra).

Suitable promoters are those promoters known to induce transcription of a gene in the host cell. The promoters may be inducible promoters and/or those that are constitutively expressed in the host cell of interest.

In some embodiments, the cloning vectors useful in the present technology can contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for either or both prokaryotic and eukaryotic systems. Vectors for use in the present technology can be suitable for replication and integration in prokaryotes, eukaryotes, or both. See, Giliman & Smith, Gene 8:81 (1979); Roberts, et al., Nature, 328:731 (1987); Schneider, B., et al., Protein Expr. Purif. 6435:10 (1995); Ausubel; Sambrook; and Berger and Kimmel, Guide to Molecular Cloning Techniques, Methods in Enzymology volume 152 Academic Press, Inc., San Diego, Calif. A large number of suitable vectors are known in the art and/or commercially available. A catalogue of bacteria and bacteriophages useful for cloning is provided, e.g., by the American Type Culture Collection (ATCC), e.g., The ATCC Catalogue of Bacteria and Bacteriophage published yearly by the ATCC. Additional basic procedures for sequencing, cloning and other aspects of molecular biology and underlying theoretical considerations are also found in Watson et al. (1992) Recombinant DNA Second Edition, Scientific American Books, NY.

In some embodiments, an expression vector can be introduced into the host cell by any of the variety of techniques well known in the art, including, e.g., electroporation, calcium phosphate precipitation, lipid mediated transfection (lipofection), biolistic delivery, or the like. Expression is optionally constitutive or inducible, as desired. The cell is optionally used for in vivo synthesis of a methylketone produced by action of the expressed MKS2 protein alone or in coordination with the host fatty acid biosynthetic enzymes downstream. In some embodiments, an extract or lysate from the host cell can be used for in vitro production of the methylketone (or a methylketone metabolite). In still other embodiments, a MKS2 polypeptide can be purified from the host cell.

The host cell, can optionally, include a cell that does not naturally produce ShMKS2 or SlMKS2, such as E. coli. One or more additional fatty acid biosynthetic enzymes or intermediate proteins required for activity of the MKS2 can be optionally expressed in the host cell, endogenously or heterologously. Exemplary host cells can also include MKS2 gene modified (or knockout) versions of natural hosts such as Solanum lycopersicum, Solanum habrochaites or Arabidopsis sp. Exemplary host cells can include, but are not limited to, prokaryotic cells such as E. coli, Pseuomonas sp. and other bacteria and eukaryotic cells such as yeast, plant, insect, amphibian, avian, and mammalian cells, including human cells. Bacteria with a higher or lower AT vs. GC content in their genomes relative to E. coli are optionally used as host cells, to optimize expression of similarly-biased genes; for example, S. coelicolor or S. lividans is optionally used for expression of GC-rich constructs (Anne and Van Mellaert (1993) “Streptomyces lividans as host for heterologous protein production” FEMS Microbiol Lett. 114(2):121-8),

Where in vivo production of methylketones (or methylketone metabolites) by the MKS2 polypeptide is desired, the precursors required for methylketone or fatty acid (or other) biosynthesis can be endogenous to the cell, such precursors can be provided exogenously and taken up by the cell, and/or biosynthetic pathway(s) to create the precursors in vivo can be generated in the host cell.

A host cell expressing a methylketone synthase polypeptide for production of alkyl methylketones having a carbon backbone ranging from C₇ to C₂₀ can also optionally expresses one or more additional enzymes, for example, methylketone synthase 1 enzyme (MKS1) whose collective action assists in the decarboxylation of a 3-ketoacyl intermediate product into a final product with the activity provided by MKS2. Any such downstream enzymes can be expressed endogenously and/or heterologously.

Additional new enzymes expressed in the host cell (e.g., for MKS2 activity, precursor synthesis, and/or downstream tailoring enzymes) are optionally naturally occurring enzymes, e.g., from other species, or artificially evolved enzymes. The genes for these enzymes can be introduced into a cell by transforming the cell with a plasmid comprising the genes and/or integrating the genes into the host's genome. The genes, when expressed in the cell, provide an enzymatic pathway to synthesize the methylketone compound. Examples of the types of enzymes that are optionally added are provided herein, and additional enzyme sequences can be found, e.g., in Genbank and in the literature.

Any of a variety of methods can be used for producing novel enzymes, e.g., for use in biosynthetic pathways or for evolution of existing pathways, in vitro or in vivo. Many available methods of evolving enzymes and other biosynthetic pathway components can be applied to the present invention to produce precursors or products (or, indeed, to evolve synthases or domains thereof to have new substrate specificities or other activities of interest). For example, DNA shuffling is optionally used to develop novel enzymes and/or pathways of such enzymes for the production of precursors or products (or production of new synthases), in vitro or in vivo. See, e.g., Stemmer (1994) “Rapid evolution of a protein in vitro by DNA shuffling” Nature 370(4):389-391; and, Stemmer, (1994) “DNA shuffling by random fragmentation and reassembly: In vitro recombination for molecular evolution” Proc. Natl. Acad. Sci. USA., 91:10747-10751. A related approach shuffles families of related (e.g., homologous) genes to quickly evolve enzymes with desired characteristics. An example of such “family gene shuffling” methods is found in Crameri et al. (1998) “DNA shuffling of a family of genes from diverse species accelerates directed evolution” Nature, 391(6664):288-291. In yet another approach, random or semi-random mutagenesis using doped or degenerate oligonucleotides for enzyme and/or pathway component engineering can be used, e.g., by using the general mutagenesis methods of e.g., Arkin and Youvan (1992) “Optimizing nucleotide mixtures to encode specific subsets of amino acids for semi-random mutagenesis” Biotechnology 10:297-300; or Reidhaar-Olson'et al. (1991) “Random mutagenesis of protein sequences using oligonucleotide cassettes” Methods Enzymol. 208:564-86. Yet another approach, often termed a “non-stochastic” mutagenesis, which uses polynucleotide reassembly and site-saturation mutagenesis can be used to produce enzymes and/or pathway components, which can then be screened for an ability to perform one or more methylketone synthase or fatty acid biosynthetic pathway function (e.g., for the production of precursors or products in vivo). See, e.g., Short “Non-Stochastic Generation of Genetic Vaccines and Enzymes” WO 00/46344.

Other useful references, e.g. for cell isolation and culture (e.g., for subsequent nucleic acid or polypeptide isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell Tissue and Organ Culture; Fundamental Methods Springer Lab Manual, Springer-Verlag (Berlin Heidelberg New York) and Atlas and Parks (eds) “The Handbook of Microbiological Media” (1993) CRC Press, Boca Raton, Fla.

A variety of protein isolation and detection methods are known and can be used to isolate polypeptides of the present technology, e.g., from recombinant cultures of cells expressing the recombinant MKS2 proteins of the present technology where such purification is desired. A variety of protein isolation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2.sup.nd Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3.sup.rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000). The fusion protein optionally includes a tag to facilitate purification, e.g., a GST, polyhistidine, and/or S tag. The tag(s) are optionally removed by digestion with an appropriate protease (e.g., thrombin or enterokinase).

The example embodiments, including the materials and methods, described herein are exemplary and not intended to be limiting in describing the full scope of compositions and methods of the present technology. Equivalent changes, modifications and variations of some embodiments, materials, compositions and methods can be made within the scope of the present technology, with substantially similar results.

### EXAMPLES, MATERIALS, AND METHODS

### Plant Material, Interspecific F2 and Backcross Populations

Solanum lycopersicum (var. M82 indeterminate) and Solanum habrochaites f glabratum (PI126449) were obtained from the Tomato Seed Stock Center at the University of California (Davis, Calif.) and from the USDA Agricultural Research Service (Ithaca, N.Y.). A single PI126449 plant served as the male to fertilize S. lycopersicum. The hybrids (i) were selfed to obtain the F2 population, (ii) served as the male for fertilizing M82 to obtain BC1M82, or (iii) served as the female for a PI126449 male to obtain BC1PI. Seeds were sprouted in trays for 2 days in a closed room at 25° C. and 95% humidity and were then grown in an open greenhouse for 3 weeks. Seedlings were transplanted to the greenhouse, trellised with ropes, and grown in red loam soil with 1 m³ water and 50 mL fertilizer (Shefer, ICL Fertilizer, ISRAEL) per day. For bulk analysis, F2 plants were propagated by cuttings using rooting powder with 0.3% indole-3-butyric acid. Cuttings were rooted in germination trays held under spraying water for half an hour twice a day.

Volatile Analysis

Six young leaflets (the first, second and third from the first or second leaves) were sampled into scintillation vials on ice and volatiles were extracted and analyzed as described in Fridman et al., (2005) Metabolic, genomic, and biochemical analyses of glandular trichomes from the wild tomato species Lycopersicon hirsutum identify a key enzyme in the biosynthesis of methylketones, Plant Cell 17: 1252-1267.

Morphology Indexes

Six young leaflets (opposite those taken for volatile analysis) were sampled into scintillation vials and a digital photo of the central upper surface was taken. Mean trichome number per square millimeter was calculated and trichome shape was classified as follows: wild shape (PI shape), intermediate shape (intermediate) and cultivated-like shape (M82-like shape).

Genotyping

DNA samples were extracted from approx. 100 mg of fresh young tomato leaves and buds following the protocol described by Murray and Thompson (1980). See PCR conditions and primers in the corresponding sections herein. KAS I PCR products (15 μL) were digested with 1 μL TaqI restriction enzyme (New England Biolabs, Ipswich, Mass.) for 1 h at 65° C. in a reaction that included 2 μL 10× buffer and 10 μg bovine serum albumin (BSA; 20 μL total).

High Resolution Melt (HRM) Genotyping

Sequences were aligned using the Align function in the Vector NTI software package (Invitrogen Corporation, Carlsbad, Calif.) to identify single-nucleotide polymorphisms (SNPs) between the sequences of the S. lycopersicum and S. habrochaites alleles. The S. habrochaites sequences were taken from an EST library produced from the glandular trichomes of accession PI126449 and the S. lycopersicum alleles were retrieved from the total tomato EST repository (SOL database, available online at www.sgn.cornell.edu/index.pl). Three different pairs of primers flanking the identified SNPs (amplicon size varied from 60 to 100 by per SNP) were selected for each gene using the primer3 software (available online at primer3.sourceforge.net). First, PCR was conducted with a test panel that included the parental lines M82 and PI, and their hybrid (F1). PCR products were analyzed on an agarose gel (3%) and reactions that produced a single product with no primer dimers were selected for HRM analysis on a Rotor-Gene 6000 (Corbett Research, Sydney, Australia). Primers that showed the best allelic discrimination by HRM examination were selected to score the genotype of the F2 population. HRM was performed immediately after the PCR cycles as a single run following the manufacturer's default parameters.

Transcriptome Analysis

Trichome isolation was performed as described in Fridman et al. (2005) Metabolic, genomic, and biochemical analyses of glandular trichomes from the wild tomato species Lycopersicon hirsutum identify a key enzyme in the biosynthesis of methylketones, Plant Cell 17:1252-1267. The tomato microarray design, cDNA synthesis, hybridization and analysis were performed by Genotypic Technology (Bangalore, India). The microarray was a complex of 44,000 probes of 25 by each, representing all the tomato ESTs (Tomato Gene Index, available online at compbio.dfci.harvard.edu/tgi/cgi-bin/tgi/gimain.pl?gudb=tomato). Total trichome RNA (5 μg) was labeled with Cy3 and Cy5 and hybridization was repeated four times (two repeats for each dye swaps between RNA samples) in a 4×44 format following the v5.5.× protocol for a two-colors array. Results were analyzed following the GEv5_{—}95_Feb07 protocol (Agilent, Santa Clara, Calif.). More details can be found at NCBI GEO under GSE16431.

Quantitative RT-PCR

Total RNA was isolated from isolated glandular trichomes as previously described (Fridman et al., (2005) Metabolic, genomic, and biochemical analyses of glandular trichomes from the wild tomato species Lycopersicon hirsutum identify a key enzyme in the biosynthesis of methylketones. Plant Cell 17: 1252-1267). The RNA was subjected to DNase treatment using a DNA-free kit (Ambion, Austin, Tex.) and first-strand cDNA was synthesized by Superscript II reverse transcriptase (Invitrogen) with poly-T primers in parallel with a negative control reaction in which no Superscript II reverse transcriptase was added. The qPCRs utilizing power SYBR-Green PCR master mix (Applied Biosystems, Foster City, Calif.), gene-specific primers, and a dilution series of each cDNA, were performed as previously described (Varbanova et al., 2007). qPCR was performed using the StepOnePlus Real Time PCR System (Applied Biosystems) and the conditions were as follows: 95° C. for 3 min, 50 cycles of 95° C. for 15 s, 60° C. for 30 s, and 72° C. for 30 s, followed by a melting cycle of 55 to 95° C. with an increasing gradient of 0.5° C., and a 10 s pause at each temperature. All reactions were performed in triplicate, and each experiment was repeated twice. ShMKS2 and SlMKS2 allele-specific primers were designed as follows:

ShMKS2 forward,
(SEQ ID NO: 40)
5′-GCCTATATTGGAGGCAAGAGGA-3′;
ShMKS2 reverse,
(SEQ ID NO: 41)
5′-TGTACACCGCAACTCTTCTGGT-3′;
SlMKS2 forward,
(SEQ ID NO: 42)
5′-ATGCAAGTTATTGCCAACATGG-3′;
SlMKS2 reverse,
(SEQ ID NO: 43)
5′-GAAAAACAAACGAGCAGCTGAA-3′;
ACC forward,
(SEQ ID NO: 44)
5′-CTGCTAGGAAAGCTCATCGTATGG-3′;
ACC reverse,
(SEQ ID NO: 45)
5′-GTGGTAGGAACTCCAGTGATAACG-3′;
MaCoA-ACP trans forward,
(SEQ ID NO: 46)
5′-GAATGACGGTACGTCTAGCTGTTG-3′;
MaCoA-ACP trans reverse,
(SEQ ID NO: 47)
5′-GGTGAAGTCACCTGGCTAGCTAAT-3′;

Actin transcript amplification was used as an internal control, with the forward primer 5′-AACACCCTGTTCTCCTGACTGA-3′ (SEQ ID NO: 48) and reverse primer 5′-AACACCATCACCAGAGTCCAAC-3′ (SEQ ID NO: 49).

Sequence Analysis

Alignment of multiple protein sequences was performed using the ClustalW program (Thompson et al., (1997) The CLUSTAL_X windows interface: flexible strategies for multiple sequence alignment aided by quality analysis tools, Nucl Acids Res 25: 4876-4882).

Statistical Analysis

Statistical analyses were conducted with JMP software (SAS Institute, Cary, N.C.). Since phenotypic data of the segregating F2 and BC did not fit the normal distribution, they were log-transformed. A non-parametric test (Wilcoxon) was used to test the MKS1 genotype effect on 2TD levels of 221 plants under the “Fit Y by X” function (because of unequal variances). The association between trichome shape and the 2TD levels was tested for 164 plants by ANOVA under the “Fit Y by X” function. For these two tests, the tested factor was set as a character and the 2TD levels were continuous. Association between the MKS1 locus genotype and trichome shape was tested in 134 plants by Pearson test for category parameters under the “Fit Y by X” function. Data of 122 individuals were used for multiple regression analysis that performed by choosing the “stepwise” option in the “Fit Model” function and the “Forward” direction was used for building the final regression model. All factors included in the analysis were set as continuous. Power was calculated with “G*Power 3.1.0” software (Faul et al. 2007). Regression analysis, including the MKS1 and MKS2 interaction, was performed by replacing these two singular factors with a new factor representing the haplotype at those loci. Interactions between genes were tested by twoway ANOVA under the “Fit Model” function.

Isolation of Full-Length ShMKS2 and SlMKS2 cDNAs and Expression in E. coli

The following primers were used to amplify the full ORF of MKS2 from PI126449 (ShMKS2) or M82 (SlMKS2) leaf cDNA into the TA cloning vector (pCRT7/CT TOPOTA; Invitrogen).

forward,
(SEQ ID NO: 50)
5′-ATGAGTGATCAGGTCTATCACC-3′;
and
reverse,
(SEQ ID NO: 51)
5′-CTCTTGATCTGGAAGCTTGA-3′.

The sequence of these cDNAs was verified and transferred into the E. coli expression vector pHis9GW following Auldridge et al. (submitted). The pHis9GW vectors carrying ShMKS2 or SlMKS2 were mobilized into E. coli BL21(DES) cells, and gene expression was induced by the addition of 2 mM IPTG after the culture OD595 value had reached 0.6-0.7. After IPTG addition, ShMKS2- and SlMKS2-expressing bacterial cells were grown at 30° C. or 18° C. overnight.

Headspace Analysis of Spent Media of E. coli Cultures Expressing ShMKS2 and SlMKS2

After induction with IPTG and growth overnight, 1 mL of culture was placed in a glass vial at 42° C. The vial was capped with a screw cap in which a small hole had been bored. The needle of a SPME device was inserted into the vial through the hole in the cap and the fiber extended for 30 min. for volatile collection, after which the fiber was withdrawn and then injected into the GC. GC-MS analysis was performed as described previously (Fridman et al., (2005) Metabolic, genomic, and biochemical analyses of glandular trichomes from the wild tomato species Lycopersicon hirsutum identify a key enzyme in the biosynthesis of methylketones. Plant Cell 17: 1252-1267). Labeled peaks in FIG. 8 were identified by comparison of retention time and MS of authentic standards (methylketones) or MS and Kovac indices (alcohols).

Homology Modeling

The MKS2 homology model was constructed using MODELLER (SalI and Blundell, 1993), and the illustration was prepared using MOLSCRIPT (Kraulis, 1991) with final rendering by POV-Ray (Persistence of Vision Ray tracer; available online at www.povray.org).

Primers and PCR Conditions.

Approximately 50-100 ng DNA was used as a template for a 25-1 μL reaction containing 0.4 μM forward and reverse primers, 0.625 units of Taq DNA polymerase (Peqlab Sawady, Erlangen, Germany), 2.5 μL of 10×PCR buffer S, and 17 μL DDW. The following reaction profile was used: 60 s at 94° C., 35 cycles of 20 s at 94° C., 20 s at Tm° C., 30 s at 68° C., and a final extension for 10 min at 68° C.

TABLE III
Primers and PCR conditions.

| Gene | Forward Primer | Reverse Primer | Tm ° C. |
|---|---|---|---|
| ACP1 | TCGCCATTTGTTAAGAAGCACTTTG | TCAGACCCCTCGATCTCTTTCAC | 58 |
| | (SEQ ID NO: 52) | (SEQ ID NO: 53) | |
| KAS I | TCGCCATTTGTTAAGAAGCACTTTG | TCAGACCCCTCGATCTCTTTCAC | 55 |
| | (SEQ ID NO: 54) | (SEQ ID NO: 55) | |

Primers and HRM Conditions

Approximately 250-500 ng DNA were used as template for a 25-1 μL reaction containing 0.4 μM forward and reverse primers, 1 unit of Taq DNA polymerase, 2.5 μL of 10×PCR buffer S, 1.5 μM syto9 (Invitrogen) and 14 μL DDW. The reaction profile was: 60 s at 94° C., 35 cycles of 20 s at 94° C., 20 s at Tm, 30 s at 68° C. Temperature was raised by increments of 0.1° C.

TABLE IV
Primers and HRM conditions.

| Gene | Forward Primer | Reverse Primer | Tm ° C. | HRM ° C. |
|---|---|---|---|---|
| Acetyl-CoA | CAATGCCAATGCTTAATTATTCTTC | TCAAGTTCCAATGAGAGTAATGTTC | 55 | 65-85 |
| carboxylase | (SEQ ID NO: 56) | (SEQ ID NO: 57) | | |
| Malonyl-CoA:ACP | ATCCGCGCTCATTATGCTAC | TGAAAGCTGGGCAGAGAAAT | 60 | 72-85 |
| transacylase | (SEQ ID NO: 58) | (SEQ ID NO: 59) | | |
| 3-Ketoacyl-ACP | TGCTGTGAAGTTTGGGTCTG | TGAGGCTTTGAGAGGTTTCTTC | 60 | 68-84 |
| synthase III | (SEQ ID NO: 60) | (SEQ ID NO: 61) | | |
| Enoyl-ACP | GAGCACTATGAGTTTCAATTTTGG | GAAGCTATGGATTGGCTTCG | 60 | 73-80 |
| reductase | (SEQ ID NO: 62) | (SEQ ID NO: 63) | | |
| ACP2 | AGGCACCTAACCGTGTATCG | TGGCTGGATTCACTCTGATG | 60 | 65-85 |
| | (SEQ ID NO: 64) | (SEQ ID NO: 65) | | |
| MKS1 | TAAGCGAGTGTTCATTGTTG | CGATCTCTTTCACTTCATCA | 56 | 65-85 |
| | (SEQ ID NO: 66) | (SEQ ID NO: 67) | | |
| MKS2 | TGGAGGCAAGAGGAATAGCA | CAAATGTGGTTAGACATTACAAGCA | 60 | 70-84 |
| | (SEQ ID NO: 68) | (SEQ ID NO: 69) | | |

Accession Numbers

Sequence data have been deposited with the GenBank data library under the following accession numbers: ShMKS2 from S. habrochaites f glabratum (accession PI126449), EU883793, and from S. lycopersicum (var. M82), EU908050. GEO accession number for raw microarray data and platform description: GSE16431.

Bioinformatics

Homologs of ShMKS1 and ShMKS2 were identified by BLAST search of the “Tomato WGS Scaffolds Prelease (previous)” data set (available online at solgenomics.net/). The genomic sequences identified in this search were checked (by BLAST) with the EST database (available online at bioinfo.bch.msu.edu/trichome_est) from the trichomes of S. lycopersicum. The positions of exons were determined by comparisons with ESTs directly derived from these genes or, in the absence of ESTs, from a comparison with ShMKS1 and ShMKS2 cDNAs, respectively. Protein sequence comparisons were performed with the CLUSTAL_X protocol (Thompson et al., (1997) The CLUSTAL_X windows interface: flexible strategies for multiple sequence alignment aided by quality analysis tools, Nucl Acids Res 25: 4876-4882).

Gene Isolation

A full-length cDNA of ShMKS2 was isolated by RT-PCR using the oligonucleotides 5′-ATGTCTCATTCGTTCAGCA-3′ (SEQ ID NO: 70) and 5′-GAGATGATGTTGTACACCGCAACT-3′ (SEQ ID NO: 71) (oligonucleotides 1 and 4, FIG. 30) with total RNA from S. habrochaites. The genomic sequence of ShMKS2 was obtained using total DNA as the templates. The promoter sequence of ShMKS2 was isolated by PCR using the oligonucleotides 5′-CTGTGGCAATTGTTAATTGGTGGGAGT-3′ (SEQ ID NO: 72) (oligonucleotide 1, FIGS. 29) and 5′-GAGCGGGAGTTGCCGGTGAG-3′ (SEQ ID NO: 73) (oligonucleotide 2, FIG. 30). The genomic sequence of ShMKS1 was obtained by PCR with nucleotides 5′-ATGGAGAAAAGCATGTCGCCA-3′ (SEQ ID NO: 74) and 5′-TTTATACTTGTTAGCGATGCTTAGAAGAGT-3′ (SEQ ID NO: 75) (oligonucleotides 1 and 2, respectively, FIG. 26). All PCR reactions employed KOD hot start polymerase (Novagen). Products were spliced into the pGEM-T easy vector (Promega) and sequenced.

5′ RACE

5′ RACE procedure used the SMART RACE cDNA amplification kit (Clontech laboratories) with SuperScript II Reverse Transcriptase and anchored Oligo(dT)20 (Invitrogen). Two independent experiments with different primers were performed for each gene with RACE-ready cDNA synthesized from total RNA from the leaves. Products were spliced into the pGEM-T easy vector (Promega) and sequenced.

Genome Walking

Isolation of the promoter region of ShMKS1 was done with the GenomeWalker Universal Kit (Clontech, Inc.) according to the manufacturer's instructions.

Constructs for subcellular localization Full-length ShMKS2 cDNA and ShMKS2 without the coding region of exon 1 (starting with the first ATG codon in exon 2) were amplified by KOD polymerase to add Bgl II and Sal I restriction sites and spliced into pSAT6A-EGFP—N1 (Tzfira et al., 2005). The expression cassettes were digested by PspI and ligated to pPZP-RCS2 binary vector and transferred into Agrobacterium tumefaciens strain EHA105 (Tzfira et al., (2005) pSAT vectors: a modular series of plasmids for autofluorescent protein tagging and expression of multiple genes in plants, Plant Mol Biol 57: 503-516).

Transient Expression in Nicotiana benthamiana and Confocal Microscopy

Agrobacterium tumefaciens cells were grown in a shaker-incubator at 30° C. at 200 rpm in LB broth supplemented with 200 μg per mL spectinomycin and 200 μg per mL streptomycin until the optical density of the culture at 600 nm reached 0.7-0.9. Bacteria were pelleted by centrifugation at 5000 rpm for 10 min at room temperature, and resuspended to OD 0.4 in fresh infiltration buffer containing 10 mM MgCl₂ and 0.1 μM acetosyringone. The resulting mix was diluted with infiltration buffer to OD of 0.1 and infiltrated into the abaxial air spaces of 4-6 week-old N. benthamiana plants by a syringe, as previously described (Yang et al, (2000) In vivo analysis of plant promoters and transcription factors by agroinfiltration of tobacco leaves, Plant Journal 22: 543-551). The plants were then returned to the growth chamber for 48-72 hours for an optimal expression of the gene.

To test for the localization of the ShMKS2 protein, the infiltrated tobacco leaves were dissected and mounted on a microscope slide with distilled water and examined using a Leica SP5 confocal system and a 63× (1.3NA) glycerin immersion lens. eGFP was visualized using an argon gas 488 nm laser, a RP500 dichroic minor, and PMT detection from 500-530 nm. Chloroplast fluorescence was visualized using the same argon gas 488 nm laser, a RP500 dichroic minor, and PMT detection from 650 nm longpass.

Expression of ShMKS1 and ShMKS2 in E. coli

The coding regions of ShMKS1 and ShMKS2 (minus the transit peptide-encoding region) were each amplified by PCR and inserted into the E. coli expression vector pEXP-TOPO-CT (Invitrogen). The expression vectors were introduced into E. coli BL21 Star (DE3) cells, and gene expression was induced by the addition of 0.5 mM IPTG after the culture optical density at 600 nm had reached 0.65. After induction with IPTG and growth at 18° C. overnight, the cells expressing ShMKS1 or ShMKS2 were centrifuged at 5000 rpm for 15 minutes, and 1-mL aliquots of the spent medium were placed in individual vials for further analysis.

GC-MS Analysis of Spent Medium of E. coli Cells Expressing ShMKS2

Aliquots (1 mL) of the spent medium of E. coli expressing ShMKS2, obtained by centrifuging the culture solution after the incubation time at 5,000 rpm for 15 min and collecting the solution without the cells, were treated in the following ways: 1. Incubated with 40 μl (3 μg) of purified MKS1 in phosphate buffer (12.5 mM NaH₂PO₄, 125 mM NaCl, 2 mM DTT, pH 6.8) for 2 hrs at 30° C. 3. Incubated at 75° C. for 30 min followed by 30 min at 30° C. 4. Incubated with 1 mL of 2M H₂SO₄ at 75° C. for 30 min followed by 30 min at 30° C. After the various treatments, 1 mL of hexane containing 5 ng/μL linalool as an internal standard was added and the resulting mixture was vortexed and centrifuged at 5000 rpm for 10 min. Two μL of the resulting extract were injected into the GC-MS for determination of methylketones. GC-MS and product analysis were performed as described by Ben-Israel et al, (2009) Multiple biochemical and morphological factors underlie the production of methylketones in tomato trichomes, Plant Physiology 151: 1952-1964.

Affinity Purification of ShMKS1 and ShMKS2

His-tagged ShMKS1 and ShMKS2 were affinity-purified on Nickel-agarose chromatography using the protocol described in Fridman et al., (2005) Metabolic, genomic, and biochemical analyses of glandular trichomes from the wild tomato species Lycopersicon hirsutum identify a key enzyme in the biosynthesis of methylketones, Plant Cell 17: 1252-1267. After elution from the Nickel-agarose column, the proteins were analyzed by SDS-PAGE and ShMKS2 dialyzed against 50 mM phosphate buffer pH 6.8, 500 mM NaCl, and 1M (NH₄)₂SO₄, and 2 mM DTT and ShMKS1 dialyzed against 12.5 mM phosphate buffer pH 6.8, 50 mM NaCl, and 2 mM DTT. ShMKS1 purity was estimated at 99%, and ShMKS2 purity was estimated at 6%.

Decarboxylase Activity Assays

A typical decarboxylase assay consisted of a 500 μL reaction solution containing ShMKS1 or ShMKS2 (2.5 μg), 3-ketomyristic acid (0.1 mM), and 1,3-bis(tris(hydroxymethyl)methylamino) propane—Na⁺ (20 mM, pH 7.0). For measuring kinetic parameters, substrate concentrations ranged from 5 μM to 75 μM. Assays were performed at 23° C. for 10 min after addition of protein. Reactions were quenched by addition of 25 μL, 3 M NaOH to ensure any remaining 3-ketoacid was anionic and unlikely to be extracted by hexane. Omission of the base neutralization step resulted in the extraction of free 3-ketoacids and spontaneous decarboxylation upon heating in the GC-MS's inlet. Methylketone products were extracted with 500 μL hexane. For reaction normalization, a standard concentration of 2-undecanone was added prior to extraction to a final concentration of 4 μM. Reaction products (5 μL) were analyzed by a modified procedure, as described in O'Maille et al (2004) A single-vial analytical and quantitative gas chromatography-mass spectrometry assay for terpene synthases. Anal Biochem 335: 210-217, using a Hewlett-Packard 6890 gas chromatograph (GC) coupled to a 5973 mass selective detector (MSD) equipped with an HP-5MS capillary column (0.25 mm i.d. 30 m length with 0.25 μm film thickness) (Agilent Technologies). Product quantification was performed using total ion monitoring (TIM) mode where all ions in the mass spectrum contribute to the measured response. The GC was operated at a He flow rate of 1.5 mL/min, and the MSD was operated at 70 eV. Splitless injections (5 μL) were performed with an inlet temp of 280° C. The GC was programmed with an initial oven temp of 60° C. (2-min hold), which was then increased 5° C./min up to 200° C., followed by a 50° C./min ramp until 280° C. (5-min hold). A solvent delay of 8.5 min was included prior to the acquisition of the MS data. 2-Tridecanone was quantified by integration of peak areas using Enhanced Chemstation (version B.01.00, Agilent Technologies). The GC-MS instrument was calibrated with an authentic 2-undecanone standard included in the quenched reactions prior to hexane extraction.

3-Ketomyristic acid was prepared from methyl 3-oxotetradecanoate (1 mmol) by addition of 6 mL 3.0 M aqueous NaOH in addition to several drops of THF to aid in dissolution of the esterified starting material. The mixture was stirred at 23° C. for 12 hr. The mixture was then diluted with 10 mL water and acidified to pH 2-3 by adding 3 M HCl dropwise while monitoring pH. The acidified mixture was next extracted 5× with 30 mL methylene chloride. The organic phases were pooled, washed with saturated NaCl and then dried using anhydrous sodium sulfate. The methylene chloride solvent was removed under reduced pressure yielding an opaque yellowish powder. This powder was purified using a normal phase silica gel column after dissolution in a minimal amount of column solvent [methylene chloride-methanol (3:1)] to afford 3-ketomyristic acid.

Thioesterase Activity Assays

3-Ketomyristoyl-ACP was synthesized in a 500 μL reaction volume containing 1,3-bis(tris(hydroxymethyl)methylamino) propane (20 mM, pH 7.0), malonyl-CoA (0.2 mM), lauroyl-CoA (0.2 mM), ShACP (0.1 mM), EcFabD (10 μg) and MtFabH (10 μg). After 5 hr at 37° C., the in vitro reaction was used as the substrate solution for subsequent treatments. A 20 μL solution containing 2.5 μg of MKS1 or MKS2 (or buffer only) was added, and the reaction incubated for an additional 30 min at 23° C. Hexane was used for extraction either directly or after being treated with acid and heat. Hexane extracts were analyzed by GC-MS as described above. The values for heat-treated samples shown in FIG. 20 were corrected for loss of methylketones during the heating step, as determined by comparisons with standards.

Non-Limiting Discussion of Terminology

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

As used herein, the words “desire” or “desirable” refer to embodiments of the technology that afford certain benefits, under certain circumstances. However, other embodiments may also be desirable, under the same or other circumstances. Furthermore, the recitation of one or more desired embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

“A” and “an” as used herein indicate “at least one” of the item is present; a plurality of such items may be present, when possible. “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters.

When an element or layer is referred to as being “on,” “engaged to,” “connected to” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

What is claimed is:

1. An isolated polypeptide comprising an amino acid sequence that is at least 80% identical to SlMKS2 (SEQ ID NO: 3) or ShMKS2 (SEQ ID NO: 4).

2. The isolated polypeptide of claim 1, wherein the polypeptide has thioesterase activity.

3. The isolated polypeptide of claim 1, wherein the amino acid sequence is at least 95% identical to SlMKS2 (SEQ ID NO: 3) or ShMKS2 (SEQ ID NO: 4).

4. The isolated polypeptide of claim 1, wherein the amino acid sequence comprises SlMKS2 (SEQ ID NO: 3) or ShMKS2 (SEQ ID NO: 4).

5. The isolated polypeptide of claim 4, wherein the polypeptide has thioesterase activity.

6. The isolated polypeptide of claim 1, wherein the amino acid sequence is at least 95% identical to ShMKS2 (SEQ ID NO: 25), SlMKS2a (SEQ ID NO: 27), SlMKS2b (SEQ ID NO: 28), or SlMKS2c (SEQ ID NO: 26).

7. The isolated polypeptide of claim 1, wherein the amino acid sequence comprises ShMKS2 (SEQ ID NO: 25), SlMKS2a (SEQ ID NO: 27), SlMKS2b (SEQ ID NO: 28), or SlMKS2c (SEQ ID NO: 26).

8. The isolated polypeptide of claim 7, wherein the polypeptide has thioesterase activity.

9. The isolated polypeptide of claim 1, wherein the amino acid sequence is at least 95% identical to AtMKS2-1 (SEQ ID NO: 76), AtMKS2-2 (SEQ ID NO: 77), AtMKS2-3 (SEQ ID NO: 78), Rice MKS2 (SEQ ID NO: 79); Corn MKS2 (SEQ ID NO: 80); Castor bean MKS2 (SEQ ID NO: 81); or Solanum peruvianum MKS2 (SEQ ID NO: 82).

10. The isolated polypeptide of claim 1, wherein the amino acid sequence comprises AtMKS2-1 (SEQ ID NO: 76), AtMKS2-2 (SEQ ID NO: 77), AtMKS2-3 (SEQ ID NO: 78), Rice MKS2 (SEQ ID NO: 79); Corn MKS2 (SEQ ID NO: 80); Castor bean MKS2 (SEQ ID NO: 81); or Solanum peruvianum MKS2 (SEQ ID NO: 82).

11. The isolated polypeptide of claim 10, wherein the polypeptide has thioesterase activity.

12. An isolated nucleic acid that encodes the polypeptide of claim 1.

13. The isolated nucleic acid of claim 12, wherein the nucleic acid comprises a nucleotide sequence encoding a polypeptide at least 95% identical to SlMKS2 (SEQ ID NO: 3) or ShMKS2 (SEQ ID NO: 4).

14. The isolated nucleic acid of claim 12, wherein the nucleic acid comprises a nucleotide sequence encoding SlMKS2 (SEQ ID NO: 3) or ShMKS2 (SEQ ID NO: 4).

15. The isolated nucleic acid of claim 12, wherein the nucleic acid comprises a nucleotide sequence encoding a polypeptide at least 95% identical to ShMKS2 (SEQ ID NO: 25), SlMKS2a (SEQ ID NO: 27), SlMKS2b (SEQ ID NO: 28), or SlMKS2c (SEQ ID NO: 26).

16. The isolated nucleic acid of claim 12, wherein the nucleic acid comprises a nucleotide sequence encoding ShMKS2 (SEQ ID NO: 25), SlMKS2a (SEQ ID NO: 27), SlMKS2b (SEQ ID NO: 28), or SlMKS2c (SEQ ID NO: 26).

17. The isolated nucleic acid of claim 12, wherein the nucleic acid comprises a nucleotide sequence at least 95% identical to SlMKS2a (SEQ ID NO: 36), SLMKS2b (SEQ ID NO: 37), SLMKS2c (SEQ ID NO: 38), or ShMKS2 (SEQ ID NO: 39).

18. The isolated nucleic acid of claim 12, wherein the nucleic acid comprises SLMKS2a (SEQ ID NO: 36), SLMKS2b (SEQ ID NO: 37), SLMKS2c (SEQ ID NO: 38), or ShMKS2 (SEQ ID NO: 39).

19. The isolated nucleic acid of claim 12, wherein the nucleic acid comprises a nucleotide sequence encoding a polypeptide at least 95% identical to AtMKS2-1 (SEQ ID NO: 76), AtMKS2-2 (SEQ ID NO: 77), AtMKS2-3 (SEQ ID NO: 78), Rice MKS2 (SEQ ID NO: 79); Corn MKS2 (SEQ ID NO: 80); Castor bean MKS2 (SEQ ID NO: 81); or Solanum peruvianum MKS2 (SEQ ID NO: 82).

20. The isolated nucleic acid of claim 12, wherein the nucleic acid comprises a nucleotide sequence encoding AtMKS2-1 (SEQ ID NO: 76), AtMKS2-2 (SEQ ID NO: 77), AtMKS2-3 (SEQ ID NO: 78), Rice MKS2 (SEQ ID NO: 79); Corn MKS2 (SEQ ID NO: 80); Castor bean MKS2 (SEQ ID NO: 81); or Solanum peruvianum MKS2 (SEQ ID NO: 82).

21. A recombinant expression vector comprising the nucleic acid of claim 12.

22. A cell comprising the recombinant expression vector of claim 21.

23. The cell of claim 22, further comprising a Methylketone Synthase 1 (MKS1).

24. The cell of claim 23, wherein the Methylketone Synthase 1 (MKS1) comprises ShMKS1 (SEQ ID NO: 17), SlMKS1a (SEQ ID NO: 18), SlMKS1b (SEQ ID NO: 19), SlMKS1d (SEQ ID NO: 20), or SlMKS1e (SEQ ID NO: 21).

25. The cell of claim 22, wherein the recombinant expression vector is integrated into the genomic DNA of the cell.

26. The cell of claim 22, wherein the cell is a prokaryote.

27. The cell of claim 22, wherein the cell is a plant cell.

28. A multicellular organism comprising the recombinant expression vector of claim 21, wherein the recombinant expression vector is integrated into the genomic DNA of the organism.

29. The multicellular organism of claim 28, wherein the organism is a plant.

30. A method of making a methylketone or methylketone intermediate comprising:
hydrolyzing a 3-ketoacyl intermediate with a recombinant Methylketone Synthase 2 (MKS2) to form a 3-ketoacid.

31. The method of claim 30, further comprising collecting the 3-ketoacid.

32. The method of claim 30, wherein the 3-ketoacyl intermediate comprises 3-ketoacyl-ACP or 3-ketoacyl-CoA.

33. The method of claim 30, wherein the recombinant MKS2 comprises SlMKS2 (SEQ ID NO: 3) or ShMKS2 (SEQ ID NO: 4).

34. The method of claim 30, wherein the MKS2 comprises ShMKS2 (SEQ ID NO: 25), SlMKS2a (SEQ ID NO: 27), SlMKS2b (SEQ ID NO: 28), or SlMKS2c (SEQ ID NO: 26).

35. The method of claim 30, wherein the MKS2 comprises AtMKS2-1 (SEQ ID NO: 76), AtMKS2-2 (SEQ ID NO: 77), AtMKS2-3 (SEQ ID NO: 78), Rice MKS2 (SEQ ID NO: 79); Corn MKS2 (SEQ ID NO: 80); Castor bean MKS2 (SEQ ID NO: 81); or Solanum peruvianum MKS2 (SEQ ID NO: 82).

36. The method of claim 30, further comprising:
decarboxylating the 3-ketoacid to form a 2-methylketone.

37. The method of claim 36, further comprising collecting the 2-methylketone.

38. The method of claim 36, wherein the decarboxylating comprises heating the 3-ketoacid or treating the 3-ketoacid with acid and heat.

39. The method of claim 36, wherein the decarboxylating comprises decarboxylating the 3-ketoacid with a Methylketone Synthase 1 (MKS1) to form a 2-methylketone.

40. The method of claim 39, wherein the MKS1 comprises ShMKS1 (SEQ ID NO: 17), SlMKS1a (SEQ ID NO: 18), SlMKS1b (SEQ ID NO: 19), SlMKS1d (SEQ ID NO: 20), or SlMKS1e (SEQ ID NO: 21).

41. The method of claim 30, wherein the hydrolyzing occurs within or proximate to a cell expressing the recombinant MKS2.

42. The method of claim 41, further comprising isolating the 3-ketoacid from the cell.

43. The method of claim 41, further comprising
decarboxylating the 3-ketoacid with a Methylketone Synthase 1 (MKS1) to form a 2-methylketone within or proximate to the cell and the cell expresses the MKS1.

44. The method of claim 43, further comprising isolating the 2-methylketone from the cell.

45. The method of claim 30, wherein the hydrolyzing occurs within a plant and the plant expresses the recombinant MKS2.

46. The method of claim 45, further comprising
decarboxylating the 3-ketoacid with a Methylketone Synthase 1 (MKS1) to form a 2-methylketone within the plant and the plant expresses the MKS1.


SEQUENCE LISTING (82 sequences)

SEQ ID NO: 1 - protein, 207 aa, Humulus lupulus (L. cultivar Phoenix)
  MISC_FEATURE: HlGD249868
MLQTFSPSYKPLHLPISSLSLSSFSSSSASSVAFPVTRLLIPPRLRVLPNPRRRCSALPFDIRGGKGMSEFYEVELKVRDYELDQYGVVNNAVYASYCQHGRHELLESFGLSCDAVARNGDALALSELSLKFLAPLRSGDKFVVKVRISGSSAARLYFDHLIFKLPNQEPILDAKGTAVWLDKNYRPVRIPPEVRSKLVQFLRHEES

SEQ ID NO: 2 - protein, 196 aa, Glycine max
  MISC_FEATURE: GmAW394535
MSLPSPLYLNTTSFRLTRQSPFPFPRRRFNPPAFRSVSPLSSSPSASLFDLRGGKGMSGFHDVELKVRDYELDQYGVVNNAVYASYCQHGRHELLQNIGINCDAVARSGDALALSELSLKFLAPLRSGDKFVVRVRISGSSAARLYFDHFIYKLPNQEPILEAKAIAVWLDKNYRPIRIPAEMKSKFVKFIRIEDS

SEQ ID NO: 3 - protein, 141 aa, Solanum lycopersicum
  MISC_FEATURE: SlMKS2
MAEFHEVELKVRDYELDQYGVVNNAIYASYCQHGRHELLERIGISADEVARSGDALALTELSLKYLAPLRSGDRFVVKARISDSSAARLFFEHFIFKLPDQEPILEARGIAVWLNKSYRPVRIPAEFRSKFVQFLRQEASN

SEQ ID NO: 4 - protein, 149 aa, Solanum habrochaites
  MISC_FEATURE: ShMKS2
MSDQVYHHDVELTVRDYELDQFGVVNNATYASYCQHCRHAFLEKIGVSVDEVTRNGDALAVTELSLKFLAPLRSGDRFVVRARLSHFTVARLFFEHFIFKLPDQEPILEARGIAVWLNRSYRPIRIPSEFNSKFVKFLHQKSCGVQHHL

SEQ ID NO: 5 - protein, 139 aa, Petunia integrifolia subsp. Inflate
  MISC_FEATURE: PiAAS90598
MNEFYEVELKVRDYELDQYGVVNNAIYASYCQHCRHELLEKIGVNADAVARNGEALALTEMTLKYLAPLRSGDRFIVKVRISDSSAARLFFEHFIFKLPDQEPILEARGTAVWLNKSYRPVRIPSEFRSKFVQFLRQEA

SEQ ID NO: 6 - protein, 215 aa, Picea glauca
  MISC_FEATURE: PgEX412733
MATAMGAISGGISVGVNARYPHVQCSSFIQNPTKKLSRALAFPSLRTASCNPVFRRALPPIADMYNMELFGAKGMARPFELELKVRDYELDQYGVVNNATYASYCQHCRHELCEAIGFSPDAIARTGNALALSELSLKYLAPLRSGDSFVVTARISGSSAVRLFFEHFIYKLPNREPVLEAKATAVYLDKIYRPVRLPADFKSKITLFLRNEELN

SEQ ID NO: 7 - protein, 208 aa, Vitis vinifera
  MISC_FEATURE: VvCAO42155
MLQALLSPTHMAVPASRAHTRGLRLYRPPLLLPAPQPPSNCRSPRLRSVPAVRSASGLAFDFKGGKGMSGFLDVELKVRDYELDQYGVVNNAVYASYCQHGRHELLEKIGVNADAVARTGDALALSELTLKFLAPLRSGDRFVVKVRVSDSSAARLYFEHFIFKLPNEEPILEARATAVCLDKNYRPVRIPTEIRSKLVQFLRHEESH

SEQ ID NO: 8 - protein, 206 aa, Gossypium hirsutum
  MISC_FEATURE: GhDT554179
MLQASVFPAHAALPSPRPNATFLNLHRPSSSFPISPLLMPLRVPTLSTSRSFTVGALFDLKGGQGMTSFHEVELKVRDYELDQYGVVNNAVYASYCQHGRHELLESIGISCDEVARTGDSLALSELSLKFLGPLRSGDNFVVKVRVSNSSGARLYFEHFIFKMPNEVPILEAKATAVWLDKNYRPARIPPEFRSKFVQFLRCEEPS

SEQ ID NO: 9 - protein, 203 aa, Amborella trichopoda
  MISC_FEATURE: AtrFD440753
MQATWSQSVQCLAFPGRAPMAHVANNKPPHLRFSLFNPNRSPSSPPRLRLSSPISALASLDIPAGKGMTGFHEVELKVRDYELDQYGVVNNAVYASYCQHGRHELFEKMGMRADAVARTGEALALSELSLKFLGPLRSGDKFIMKVRISGFSAARFFFEHHIYKLPNHEPILEAKATGAWLDKSYRPIRIPSSFRSKFVQFVR

SEQ ID NO: 10 - protein, 190 aa, Arabidopsis thaliana
  MISC_FEATURE: AT1G68260.1
MFLQVTGTATPAMPAVVFLNSWRRPLSIPLRSVKTFKPLAFFDLKGGKGMSEFHEVELKVRDYELDQFGVVNNAVYANYCQHGRHEFLESIGINCDEVARSGEALAISELTMKFLSPLRSGDKFVVKARISGTSAARIYFDHFIFKLPNQEPILEAKGIAVWLDNKYRPVRIPSSIRSKFVHFLRQDDAV

SEQ ID NO: 11 - protein, 188 aa, Arabidopsis thaliana
  MISC_FEATURE: AT1G68280.1
MIRVTGTAAPAMSVVFPTSWRQPVMLPLRSAKTFKPHTFLDLKGGKEMSEFHEVELKVRDYELDQFGVVNNAVYANYCQHGMHEFLESIGINCDEVARSGEALAISELTMNFLAPLRSGDKFVVKVNISRTSAARIYFDHSILKLPNQEVILEAKATVVWLDNKHRPVRIPSSIRSKFVHFLRQNDTV

SEQ ID NO: 12 - protein, 189 aa, Arabidopsis thaliana
  MISC_FEATURE: AT1G35290.1
MLKATGTVAPAMHVVFPCFSSRPLILPLRSTKTFKPLSCFKQQGGKGMNGVHEIELKVRDYELDQFGVVNNAVYANYCQHGQHEFMETIGINCDEVSRSGEALAVSELTIKFLAPLRSGCKFVVKTRISGTSMTRIYFEQFIFKLPNQEPILEAKGMAVWLDKRYRPVCIPSYIRSNFGHFQRQHVVEY

SEQ ID NO: 13 - protein, 188 aa, Arabidopsis thaliana
  MISC_FEATURE: AT1G35250.1
MFQATSTGAQIMHAAFPRSWRRGHVLPLRSAKIFKPLACLELRGSTGIGGFHEIELKVRDYELDQFGVVNNAVYANYCQHGRHEFMDSIGINCNEVSRSGGALAIPELTIKFLAPLRSGCRFVVKTRISGISLVRIYFEQFIFKLPNQEPILEAKGTAVWLDNKYRPTRVPSHVRSYFGHFQCQHLVD

SEQ ID NO: 14 - protein, 210 aa, Oryza sativa
  MISC_FEATURE: OsCAE01692
MHHQIWRLLPSALSPIHAGAPRPSRPPARLGRPSPQRRRALALTHLATRRTCRLLAVSAQSASPHAGLRLDQFFEVEMKVRDYELDQYGVVNNAIYASYCQHGRHELLESVGISADAVARSGESLALSELHLKYYAPLRSGDKFVVKVRLASTKGIRMIFEHFIEKLPNRELILEAKATAVCLNKDYRPTRISPEFLSKLQFFTSEGSSS

SEQ ID NO: 15 - protein, 138 aa, Synechosystis sp. PCC6803 1,4-dihydroxy-2-naphthoyl-CoA thioesterase
  MISC_FEATURE: SsDHNACT
MGTFTYERQVYLADTDGAGVVYFNQFLQMCHEAYESWLSSEHLSLQNIISVGDFALPLVHASIDFFAPAHCGDRLLVNLTITQASAHRFCCDYEISQAESAQLLARAQTHHVCIALPERKKAPLPQPWQTAICDLDHP

SEQ ID NO: 16 - protein, 141 aa, Pseudomonas sp. (strain CBS-3)
  MISC_FEATURE: Ps4HBT
MARSITMQQRIEFGDCDPAGIVWFPNYHRWLDAASRNYFIKCGLPPWRQTVVERGIVGTPIVSCNASFVCTASYDDVLTIETCIKEWRRKSFVQRHSVSRTTPGGDVQLVMRADEIRVFAMNDGERLRAIEVPADYIELCS

SEQ ID NO: 17 - protein, 265 aa, Solanum habrochaites glabratum
  MISC_FEATURE: ShMKS1
MEKSMSPFVKKHFVLVHTAFHGAWCWYKIVALMRSSGHNVTALDLGASGINPKQALQIPNFSDYLSPLMEFMASLPANEKIILVGHALGGLAISKAMETFPEKISVAVFLSGLMPGPNIDATTVCTKAGSAVLGQLDNCVTYENGPTNPPTTLIAGPKFLATNVYHLSPIEDLALATALVRPLYLYLAEDISKEVVLSSKRYGSVKRVFIVATENDALKKEFLKLMIEKNPPDEVKEIEGSDHVTMMSKPQQLFTTLLSIANKYK

SEQ ID NO: 18 - protein, 265 aa, Solanum lycopersicum
  MISC_FEATURE: SlMKS1a
MEKSTSPFVKKHFVLVHTAFHGAWCWYKIVALMRSSGHNVTALDLGASGINPKQALEIPNFSDYSSPLMEFMASLPANEKLILVGHALGGLAISKAMETFPEKISVAVFLSGLMPGPNIDATTVYTKAASAVIGQLDNCVTYENGPTNPPTTLIAGPKFLATNVYHLSPIEDLALATALVRPFYLYLAEDISKEIVLSSKRYGSVKRVFIVATESDAFKKEFLELMIEKNPPDEVKEIEGSDHVTMMSKPQQLFTTLLSIANKYK

SEQ ID NO: 19 - protein, 269 aa, Solanum lycopersicum
  MISC_FEATURE: SlMKS1b
MEHANAIVLEPKAKKHFVLVHSACHGAWCWYKIVSLMTSSGHNVTALDLGASGINPKQALEIPHFSDYLSPLMEFMTSLPADEKVVVVGHSLGGLAISKAMETFPEKISVAVFLSGLMPGPSINASNVYTEALNAIIPQLDNRVTYDNGPTNPPTTLILGPKFLAASVYHLSSIKDLALATTLVRPFYLYRVEDVTKEIVLSRERYGSVRRVFIVTAENKSLKKDFQQLLIEKNPPDEVEEIDGSDHMPMMSKPQQLFTILLGIANKYT

SEQ ID NO: 20 - protein, 264 aa, Solanum lycopersicum
  MISC_FEATURE: SlMKS1d
MEKSASKVKKHFVLVHTLGHGAWSWYKIVALIRCSGHNVTALDLGGSGINPKQALEIPKFSDYLSPLMEFMTSLPVDEKIVLVGHSVGGLAISKAMETFPEKISVAVFLSGVMPGPNISASIVYTEAINAIIRELDNRVTYHNGSENPPTTFNLGPKFLETNAYHLSPIEDLALATTLVRPFYLYSAEDVSKEIVLSSKKYGSVKRVFIFAAKNEVVKKEFFQTMIEKNPPNEIEVIEGSDHATMTSKPQQLYTTLLNIANKYT

SEQ ID NO: 21 - protein, 265 aa, Solanum lycopersicum
  MISC_FEATURE: SlMKS1e
MDKIIESKAKKHFVLVHTLGHGAWSWYKIVALMRCSGHNVTALDLGGSGINAKQALEIPNFSDYLSPLMEFMTSLSTDEKIVLVGHSLGGLAISKAMETYPEKISVAVFLSGVMPGPNINASIVYTQTINAIIRELDNRVTYHNGPENPPTTLILGPKFLETNAYHLSPIEDLVLATTLVRPFYLYSAEDVSKEIVVSSKKYGLVKRVFIVAAENEALKKEFFQMMIEKNPPDEIEVIEGSDHATMMSKPQQLYDTLLSIANKYT

SEQ ID NO: 22 - protein, 289 aa, Populus trichocarpa
  MISC_FEATURE: PtMKS1L
MEQAKKHLVLISIFIILLNIAANKALSQPLHNPSKHFVLVHGAGHGAWCWYKLVPLLRSSGHNVTTIDLAASGIDPRQISDLQSISDYIRPLRDLLASLPPNEKVILVGHSLGGLALSQTMERLPSKISVAVFLTAVMPGPSLNISTLSQELVRRQTDMLDTRYTFDNGPNNPPTSLIFGPKYLLLRLYQLSPIEDWTLATTLMRETRLFTDQELSRDLVLTREKYGSVKRVFIIAEKDLTLEKDFQQWMIQKNPPNEVKEILGSDHMSMMSKPKELWACLQRISKKYN

SEQ ID NO: 23 - protein, 288 aa, Vitis vinifera
  MISC_FEATURE: VvMKS1L
MEARKKHMIFVSFLIFLVSSVYPMASEGRQANPVKHFVLVHGSCHGAWSWYKIVALLKSSGHKVTALDLAASGINPKQVGDLRSISWYFQPLRDFVESLPADERVVLVGHSLGGLAISQAMEKFPEKVSVAVFVTASMPGPTLNISTLNQESLRRQGPLLDSQFTYDNGPNNPPTTFSFGPLFLSLNVYQLSPTEDLALGTVLMRPVRLFIEEDMSNELMLSKKYASVKRVFIISEEDKLGKRDFQLWMIEKNPPDAVKEIKGSDHMVMISKPKELWVHLQAIAEKYS

SEQ ID NO: 24 - protein, 263 aa, Arabidopsis thaliana
  MISC_FEATURE: AtMES3
MSEEERKQHVVLVHGACHGAWCWYKVKPQLEASGHRVTAVDLAASGIDMTRSITDISTCEQYSEPLMQLMTSLPDDEKVVLVGHSLGGLSLAMAMDMFPTKISVSVFVTAMMPDTKHSPSFVWDKLRKETSREEWLDTVFTSEKPDFPSEFWIFGPEFMAKNLYQLSPVQDLELAKMLVRANPLIKKDMAERRSFSEEGYGSVTRIFIVCGKDLVSPEDYQRSMISNFPPKEVMEIKDADHMPMFSKPQQLCALLLEIANKYA

SEQ ID NO: 25 - protein, 208 aa, Solanum habrochaites glabratum
  MISC_FEATURE: ShMKS2 with transit peptide
MSHSFSIATNILLLNHGSPPSTFPVIPHRQLPLPNLRLSSRKSRSFEAHSAFDLKSTQRMSDQVYHHDVELTVRDYELDQFGVVNNATYASYCQHCRHAFLEKIGVSVDEVTRNGDALAVTELSLKFLAPLRSGDRFVVRARLSHFTVARLFFEHFIFKLPDQEPILEARGIAVWLNRSYRPIRIPSEFNSKFVKFLHQKSCGVQHHL

SEQ ID NO: 26 - protein, 208 aa, Solanum lycopersicum
  MISC_FEATURE: SlMKS2c with transit peptide
MSHSFSIAPNLMSLNHRSPPSTIPVIPHRQLPLPNLRLSSCKSRGFEAYNAFDLKGTQRMSDQVYDHDVELTVRDYELDQFGVVNNATYVSYCQHCCHEFLEKIGVSVDEVTRNGDALAVTELSFKFLAPLRSGDRFVVRARLSHSTVARLFFEHFIFKLPDQEPILEARGIAVWLNRSYRPIRIPSEFNSKFVKFLHQKSCGVQHRL

SEQ ID NO: 27 - protein, 208 aa, Solanum lycopersicum
  MISC_FEATURE: SlMKS2a with transit peptide
MSQCIASPLIRSIGSTSVGNSLLPNHRPPSTLPVSPHRQLLLPNLQLSVSKLRSFRAHAFDLKGSQGMAEFHEVELKVRDYELDQYGVVNNAIYASYCQHGRHELLERIGISADEVARSGDALALTELSLKYLAPLRSGDRFVVKARISDSSAARLFFEHFIFKLPDQEPILEARGIAVWLNKSYRPVRIPAEFRSKFVQFLRQEASN

SEQ ID NO: 28 - protein, 204 aa, Solanum lycopersicum
  MISC_FEATURE: SlMKS2b with transit peptide
MSQSIVSPLIGNNCLISLFPNRRPPSTFPVRQLHLPNLQLSASKSRSFDTNAFDLNGTRGIGDLYFHEVELKVRDYELDQFGVVNNATYASYCQHCRHEYLERIGLSVDEVCRNGDALATTEISLKYLAPLRSGDRFVVKVRLSGSTAARLYFEHFIFKLPDQEPILEARGTSVWLDKSYRPVRIPSEFRSKFDQFIHQKGSNY

SEQ ID NO: 29 - DNA, 1888 bp, Solanum lycopersicum
  misc_feature: SlMKS1a
tttttttttaaaaaaaaactaattaaaaaatcgttgccaatgatttttttttatttaaaaattgaatcaatttttcttttaaataaagcaaaaaattaaaaaaaaataaaaatttgatggaagtcgctgcttctgcagcgacttatagaatattttttttattttgaaaatcgctccgtgtggagcaatttgtttaatttttttttaaaaaaagaaaaggcaaatttaagaaatcgctgctattgcaacgatttcgctttttcaaaaggtacaagtcgctacatcgtgaacgattttaccgttttaatttttaaaaaaaattgatatacccttttggaatttttgataatttttttttacccttttaactccacactctgatatatagtgcactaatgagaattaatcacatatatagtgcactaaatcccaattaaaaacaccaattccaaaagaagtaaggtttattcaaactttgtggtagttgttcaaaaatggagaaaagcacgtcgccatttgttaagaagcactttgtgctagttcatactgcattccacggagcgtggtgctggtacaagattgtggcattgatgagatcttcaggccataatgtaacagctcttgacttgggcgcttcagggatcaaccccaaacaggcccttgaaatcccaaatttttctgattactcgagtccgctaatggagttcatggcttcactccctgcaaatgaaaaactaattctcgtaggtcatgccttaggtggactcgccatttctaaagccatggaaacctttccagaaaagatttcagttgctgtatttctcagtggtctaatgcctggtccaaatatcgatgcaaccaccgtctacactaaggtaccaattttttttccatatacattcacaaccatcgtttcgtttgaaactaattaaataaatatacttcattttaggcagctagtgcagtgataggtcaactggataattgtgttacatacgaaaatggaccaacgaatcctccaaccactctcatcgcaggtcccaagttcttggcaactaatgtttatcatctgagcccaattgaggtagcaaatatcataatacaaacataattgcagttgaaatagctcatcactgactgaactaatttaaatctcttttttttttattcttcttggtgttaggatttggcgctggccactgcactagtgaggccattttatttatatctcgcggaagatatttctaaggagatagttctttcaagcaaaagatatggatccgttaagcgagtgttcattgttgctactgaaagtgatgccttcaagaaggaatttctagaattgatgattgaaaagaatccacctgatgaagtgaaagagatcgaggggtctgaccacgtgaccatgatgtctaagccccaacaactttttactactcttcttagcatcgctaacaagtataaataaaaagctattaggatcttcttcactggcatagaaaagttcagtaagccgagatatctggacaaacaaataaagtttgaggggacaattatcacttatcatatgtcaaatctatttcattagaaaacaagatatttactcaaatattgtatgactaaaaagctcacttgttcaccccagtaaatataataagaataacctattccttctaattgcgtgagcaagcaaccgattcaaacttgcttttcaagttccatagtaaatcaatgtcatgaaaattatatactataacaaagttgcatgtcagtacatcctttaactagtacagtgaacaaaaaacattcttaaagtatttaaaatttcgtaaattccattttctgtcaaaccctgttagaaaaatagccatcgattctatagagccag

SEQ ID NO: 30 - DNA, 4499 bp, Solanum lycopersicum
  misc_feature: SlMKS1b
ggaagatgtttctaaggagatagttattacaagcaaaaggtatggatcagtttggcgagcgttcattgttgcggttgaagataaacttttgaagaaggaatttcaacatttgatgattgaaaagaatccaccagatgaagtgaaatagatccaaggctctgacacaatgatcatgaggtctaagccgcaacaactttttactactcttgtgagcattgctaacaagtatacctaagatcgagctgttgattaagaagagtaagtgtgagaataaaacttcagtcagttcacatatatgatgcatttatatgactatcttaattcttccatataagttctacagagctagttaatttgatattacagttgttatttcctttcattgtaatttcggtatttggaattgacatcaataaacaacctttatgtcctgtaactactcgatttggttcaagagcaaacagtaattaaaatcacatcaatgggaaggaggacgtaattatcataccaactgagtaatccaagtaaatcttttagaagagtacaattttattttgattgggaacaatgagaagaacactaatggtaaataaaagtttgttacaggaaataccaaattttgaaagttcattccaaataaacactaaatttgcaaacaaacgattaacttaaagcataaaaaactgtaactccatcactaaatatatttttttaactaagggccagatttggtcataaaatttttacagtaatggaaaaaattaggaatatccacaatttgaaagatttaaagaatctagaatggggagcatgaaccaggacggccggacccccctatgaatataaatagccaatgcctcagtcctcaaataatagccaacgtggggtttgcttgtgcaaggaagtaagtaagcagaaaccaaactccttgaattataaagctacctttgtaatattccatattatgtgctcatacttttaaaagaaaggaaaaaaagatatggaacatgcaaatgctattgtcttagagcctaaagctaagaagcattttgtgcttgttcattcagcatgtcatggagcctggtgctggtacaagattgtgtcattgatgacatcttcagggcataatgtcacagctcttgacttgggtgcttcagggatcaaccccaaacaggcactcgaaatcccacatttttctgattacttgagtccgctaatggagttcatgacttcacttcctgctgatgaaaaagtagttgttgtaggccatagccttggtggactcgccatttctaaagccatggaaacattcccagaaaagatttcagttgctgtatttctcagtggtctaatgcctggtccaagtatcaacgcatccaatgtctacactgaggtactcatttttcatattcccaactttaaatctgttcatttttatttaaaatctttcatatttgaaactaaatatactttattataggcactcaatgcaataatacctcaacttgataatcgcgttacatatgacaatggacctacgaatcctccaaccactcttattctaggtcccaagttcttggctgctagtgtttaccatctgagctcaattaaggtaagcaaattataatactgtcattgaaattgacttctgtcattatcagaatggcccaaattaatgtgacttcttttgtctagaatagtttatccaaaattaccaatctattcgattaatattaggcttaagtaattgataacctcttaaagttgttcatataattcacttgaacatcttaactaggagttgtacctatttgaacacctaaattgtttaaaacatgtatctattaaacacaaaacgttgatataaaaaaaaattgtgtttttcacttacctgaagcacaacacgtgaaagattcaaaacaattgtttttttttctttcattttttttattgacacttgcactataattaacttaaaaacattttttcttcattcatttacacttttttcaaaaaagaaaaaagctccacacccgtaccactcatcttcttcagagtttagtttgcaaaactttgttcatttagctctaacattgtttttctttatttttttatataaaaaaagtattatttcttcctgaagataaatgaaaatcaactatgaagaaatcatacaattcagaaatcatacaattcagatttgaaagaaaaattttcttttctatgatttatttcgaatctcataaaaataatgaaacaaatatgaagaaaaataaaaaagatgaataatttttacgtatatagattgatatttgtttatcacaattttcgaacaagtatttttactgaaatacaaatataaactcattaaatttatcaaaaataatatttgaaaatttgaatgaattaatttttaagctattagttttttttttttgtcatcgatgtagattttctttcttcttcactcttagtatgaaaatatatatctttttagaaaagttaatcttcaagttatttgaaaagaaattaggttttgtgatttgacaaaaaaaattattttgggaagaagatgaagtttgaagggggttgggaaggggtaggtaacagtacaatagattgacatttttttaaaaaaaataaatatataaaatgtgtttgaaattattttaaacatatttgggaaaaatgtctaatatacccctcaactttgtcatttagagcttatacatccctcgttataaaagtggctcatatatgcccttaccgttatacaaacggctcacatatacccctgccgttataaaatgactcacatatacccttcatttaacggaagttaaaaaattaattttaaatttatatttattacttctaatttttttttaaaattatttagggatatatatgattcttctatcaaagttcaacgtatattttaatatttttcatacataaattattttttgacttcttttattataattatttgagtttcttattcttattttgttttttctttcattccttagtttaaagtaaaaaaattaaactattttttttactgtatattgtaatttaatttcgtattcgaagaaaaaatttggttatctacaacaagttttacaagaatattagtgaaacataaataaatttgattatcaaaataataattataaattagtcattgaaacaaaaaaaaagtcaaaaaaatttgtttgacgatgattatattttttagaaaaaaataataaaaatttagagtaaaattattttttttcatttccgttagaggaaaagggtatatgtgagccatttgtttacaaataggggtatatatgaaccactttcataacaaggggtatatcagctctaaatgagaaagttaaggggtatatcagactcttctcccaacatatttttataattttggagaccattgtcacatgttatttttctattagccgtttttgccatgtcagccaagtgtatcacacattgttgaaatcaaaaaatgcctaataggaataatttttgaataggttaaagtgttcaattggtactaatctcagttaaggtgtctaagtaaaatatgcagacaactttcagagaccatcaatgacttaagtcattaacataacttaaatcacaagatcaacttgcaattattggaactgatctaaatcggggttttcccttaaatcttggcgttaggatttggcgttggccactacactagtaaggccattttatttataccgcgtggaagatgttactaaggagatagttctttcaagggaaaggtatggatcagttagacgagtgttcattgtaactgctgaaaataaaagtctgaaaaaagacttccaacagttgttgattgaaaagaatccacctgatgaagtggaagagatcgatggctctgaccacatgcccatgatgtctaagccccaacaactttttaccattcttctgggcattgccaacaagtatacctaattagagctgttgatgatgaagaataaggggtcatttgtaaggctgagaacagagcttcagtcaattaacatatatgtgatgcattttatatatggccatcttagttttttcatgggaattctaaaaccttgctagttaatttgatatttcaatcattttcttttgattgttaatttctttcattgtaatttcagtgtttggctccatcaataaacagcctctatttcctgaaattactgaattttagtttcaatttgaataaatctcataaatgggaagaggatacacagatgttgatactgaagaatcagaacaatatgaatgaaaaatatgatgtagatggacaagaatttattgaggccatttggtaagaaaaagaagtatgtagtactagtggactttggagcaacacacaacattatagatgaaagagaaaaaaattagagcaaaagtattatttagtactgcacaatctatccttgtgcttgaggtttgacatc

SEQ ID NO: 31 - DNA, 2628 bp, Solanum lycopersicum
  misc_feature: SlMKS1d
cattatagaaaatactataaactctattatgatcatcaaatacctagttttcgctagctctttttttcttcatgcttgttaagtaatagagttttattattgcaataggatcggtggtctagcctcccttacttgtactctttaataaaaatttcttagtttaaaaaaactttataatcaatataaagacattttgatacgtaatcatttcaattttgagcgtggggcttcacgtacacgtataataatagctcttatgtgactttagtagtttatccacaatataacacttctcatcttttcttacacactttttctcgtatctaataaagattaagaccacactatttatataggtaaattagataaatgacttaaatgttacataaattacaaaatataataaggtaaaaaaatacatgaaatgaaaaatgggaggccataggagttgtaaggaaaataaaagaccactttataccccacaataataggcatcatgtggtcatcatcgtcgatgaaaatgcaacacaatgcaacattcaaaaacactcatactttggatcattgttatccattatattgtattgtatcgttattatatttataacgtttgttttgattgttccttaaattgtattgtattgtattgttaaatttcattgttacgtaacaatgaaaatccctattttatgaaacacccaatttggtgtgttcccattgttacttaacttttttttccaattatatatttacataatatttcaaaatactactttaccctttatcttaattatttaaatctaatcaaacctcctaccctagaataattaagaatattttagtaaatttataaattgcaatacagtatgatacgatcaaatcaaacaattaaaatgttattaaacaaaaacaatctatacagtctagtcaaacattgtatctaccatacaatacaatacaatagagtaattcgataaaattagcacacgtattctcacaaatgttttccttttccactacaaaaaattcagtatttgctactataaaaaggaagagaagaattctcaaagaatttactcatcagccaaatagtttgttttcctgtcttgtggtaattatattttgttgtacatgttacttttgtagagaagacttgttcagttctaaggaagcattttacatagttgatatggagaaaagcgcgtctaaagttaagaaacactttgtgcttgttcatacgctaggccatggagcctggtcctggtacaaaattgtagcactcataagatgttcaggacataatgtcacagctctagacttgggtggttcaggaatcaacccgaaacaggccctcgaaattccaaaattttctgattacttgagtccgctaatggagttcatgacttcacttcctgttgatgaaaaaatagttcttgttggccatagcgttggtggactcgccatttctaaagccatggaaaccttccctgaaaagatttctgttgctgtatttcttagtggtgtaatgcctggtccaaatattagtgcatcaatcgtctatactgaggtaaacatcgattttccatatttgtttacatatttgaaattaaatttgtagatatactttactataggcaatcaatgcaataatacgtgaacttgataatcgggttacataccacaacggatctgagaatcctccaacgaccttcaacctaggtcccaagttcttggaaactaatgcttaccatctgagcccaattgaggtaagcaaatcataatagtactgttactgaaattaataacaaaatttggaactgatttaaattggagttttccctaggatttggcgctggctactacactagtaaggccattttatttatacagtgcggaagatgtttctaaagagatagtactttcaagcaaaaaatatggatcagttaagagagtgttcatctttgctgctaaaaatgaagttgtgaagaaggaatttttccaaacgatgattgaaaagaatccaccaaatgaaatagaagtaatcgaggggtctgaccatgcgaccatgacgtctaagccccaacagctttatactactcttctcaacattgccaacaagtatacctgagccctttcaatttgatatttcaatcattttctttacattataatttcagtgttcgaaattagcatcaataaataacctcttatttcctgaaattactgaattcttagttcaatttgattgaatctcatcaatcggaagagcatacaaatgttaatactgataacttagacttctagatgaaataatcataaagttaaacactgcttacacaagatgatagtagcatcaagtggaggttgtactttggcaaaccaagaagctaaaacacaagcttagccttctcaatcactacaacaaaaacaacttttaaggtttataaatattcacattaacaaaaagtggtaaagtctttaacgacattatctccctacaaggattgacattatctctctacaaggattgacgtatcatcaaaattaatgatctatatattgttgttcatagtctcatgcagggccctcttccctctcttagtagtgctttgttgggctctaatatag

SEQ ID NO: 32 - DNA, 2451 bp, Solanum lycopersicum
  misc_feature: SlMKS1e
aaagcaaaataaattctttcatttatttcgaacttaatttatagaagattattttcaaatcctactaatataccgcacggttaagttatctagttcaacataatgatgagaatgtaactataatctcttcatttactaaaaaataattttatgtatcacaaaatttttgaactgagagcattaaagtaatattgtattgcttcacttctaaattactcggttcacctacatacttaatgtaatagatatggaatcacatggtagaacggtcttataatgaaataatttttgcaacttgtcaacttttttacattttgatatatcatcgtttcaattttgagcttggagcttcacggacacacattcaatatagaataagaccacactatttatgtaagtgagtaattcctttttgctgtgaatataaaaataaaattggacaaaagaattagacttaaatgtatctaaattgaattaaaagatataatgaaataaaatgaaaagttggaggttatatgagttgtaagggaaataaaagaccaccttatgccttacaataataggcattggtgcattacatgtcaccaattatgtcagtgcaaaatgcaacatcaaaaaatactttaggataagttactttaataaaaagtaattacattaaaatactcatatccctggctcaagataaagtttgcacgtgatttcacaaagataaagttgttagcaattgtattctcacaaatattttcctttttctacttcatcaaattaagtatttgctactataaaaggaagaattgtcagagaatacaagaccaaatcagcagtcaaatagtttgtcttcgtgttttgttggtttaatagaggaaaaattctgaaatagtttcttaaaatggtgtctagcacaattcaagcattgctcatacttaggtatgaatatatgataattatataattgttgttattatatatactcatattgccaccaacaaatataaaaatggacaaaattattgagtctaaagctaagaagcactttgtccttgttcataccctaggccatggagcctggtcctggtacaagattgttgcattgatgagatgttcaggacataatgtcacagctctagacttgggtggttcaggaatcaatgcgaaacaggctctcgaaatcccaaatttttctgattacttgagtccgctaatggagttcatgacttcactttctactgatgaaaaaatagttcttgtaggccatagccttggtggactcgccatctctaaagccatggaaacctatcctgaaaagatttctgttgctgtatttcttagtggtgtaatgcctggtccaaatatcaatgcatcaatcgtctacactcaggtaaacatcgattttccatatttgtttacattttttgcatatttgaaactaaatttataagatacttcatcatagacaatcaatgcaataatacgtgagcttgataatcgggttacataccacaacggacctgagaatcctccaacgactctcatcctaggtcccaagttcttggaaactaatgcttaccatctgagcccaattgaggtaagcaaatcataatagtactgttactgaaattaataacaaaatttggaactgatttaaattggggtttttccctaggatttggtgctggccactacattagtaaggccattttatttatacagtgcggaagatgtttctaaggagatagtagtttcaagcaaaaaatatggattagttaagcgagtattcattgttgctgctgaaaatgaagctctgaagaaggaatttttccaaatgatgattgaaaagaatccaccagatgaaatagaagtgatcgaggggtctgaccacgcgaccatgatgtctaagccccaacagctttatgatactcttctcagcattgccaacaagtatacctgagaccttccaatttgatgtttcaattatttcctttacattgtaatttcagtgttcggaattagcatcaataaataaactttatttgctgaaattactgaattctttagttcaatttgattaaatctcatcaatgggaagaggatacatatgttaatgttgataacttaagcttttagataaaatgatcacaaaatataacattgcttacacaagacatagtagcatcatgtgaaatgcatgtggtaggttgtactttggcaaaccaagaagctaaaacacaagcataaccttctcaatcaccacaacaaaacaactttttaaggtttataaatatgcacattaacaaacagtgttgaagtctttatcgatattatctttctacaaacagtgttgaagtctttatcgatattatctttctacaagcagtgttgaagtctttatccatcgactattcgtctcattacactgtatcaaactactaaaatcgtcataatttttta

SEQ ID NO: 33 - DNA, 2643 bp, Solanum lycopersicum
  misc_feature: SlMKS1c
taatgggcttcactccctgctgataaaaaagtacttcttctaggtcatagctttggtggactcggcatttctagagccatggaagatgtttctaaggagatagttctttcaagcaaaagatatggatcagttaggcgagtgttcattgttgctgctaaagataaatttcaaaagaaggaatttcgcctacaaatgattgaaaagaatccgcctgatgaagtgaaagagatacagggctctgaccacaggaccatcatgtctaagccccaacaactttacatcactcttctgagcattgccaacaagtataacaaatttacctagctagttaaatttgacatttaaataattttgtttgattgtaattttagtttttggaattgacctcaataagcaacctctatttcatgaaattactgaattttagttcaactctgttaaatctcatcgactggaagaggaaatagacattgatgctgaagaattagaactatatgaataaatattgcaggaaatggtcatagtagtagatggcaaatatatgataagttattcagaatgtaaggagttcatgtgagccaaaacattaaaattatagccattctcatgactccctccctggacccttttctcttaaagctcttacttttctcaacaattaaatcatttaaattgaacggagactcgtgattagaacaactgtaggacaaggtaagtcaagttcctttttactttttatttatcaccattactgtttattatgcacgaaatcataaagtatgaaaccgattagtgaactgatctactggtccaagcatgactactaatagtgaccagacttgcaccagcttctactacattgcttgcccacttcctcaaataatagccaactttaggttgcttctacgtgtaagtgaagaaaccaaactcctccaactaaaaagctgccatgtaatgtttcaaattatgtgctcatttttcagaagaaggaaaaaaatatatggagaaaagcaagtttcaaacaagtctagtagttctaatacttttgttgccttatgctaaatcaaccttgtcagggcctaaatctaagaagcactttgtactagtgcatactgcaggccacggagcctggacctggtacaagagtgtggcattgatgagatcttctgggcataatgtcacagctttggacttgggcgcttcagggatcaaccccaaacaggctcttgaaatcccaaatttttctgattatttgagtccgctaatggaatttatggcttcacttcctgctgataaaaaagtagttcttgtaggccatagctttggtggactcgccatttctaaagccatggaaacctttccagaaaagatttcggttgctgtattcgtcactgctcatatgcctggtccaaatatcaatgttgccacaatctacactgaggtagcaatgttccatatttcccataattagcaatgagatgatctgttatgtttatgcagctagcttttacatgtttgaaactaaaaatactttgttacagttattcaagtcaatatctcaacctgataatcgtattatatacgataatgggcctacaaatcctccaaccacctacatcctaggtccaaagtacatggaaactgatgtttaccagcggagcccaactcaggtaagcaaagcataattcaaatgtagtatatagccaataagtttcgttattctggtcatgagaatggccctaaagtgactcctttcgccaaaaagttactgatctagtattcaggtacttaatggcgacaataacttagagctgcatggtttaatatataatcggggttttttccttgttggtgttaggatttggcgttggcctccacactagtaaggccaataaacttctacagtctggaagatgtttctaaggagatagttattacaagcaaaaggtatggatcagtttggcgagcgttcattgttgcggttgaagataaacttttgaagaaggaatttcaacatttgatgattgaaaagaatccaccagatgaagtgaaatagatccaaggctctgacacaatgatcatgaggtctaagccgcaacaactttttactactcttgtgagcattgctaacaagtatacctaagatcgagctgttgattaagaagagtaagtgtgagaataaaacttcagtcagttcacatatatgatgcatttatatgactatcttaattcttccatataagttctacagagctagttaatttgatattacagttgttatttcctttcattgtaatttcggtatttggaattgacatcaataaacaacctttatgtcctgtaactactcgatttggttcaagagcaaacagtaattaaaatcacatcaatgggaaggaggacgtaattatcataccaactgagtaatccaagtaaatcttttagaagagtacaattttattttgattgggaacaatgagaagaacactaatggtaaataaaagtttgttacaggaaataccaaattttgaaagttcattccaaataaacactaaatttgcaaacaaacgattaacttaaagcataaaaaactgtaactccatcactaaatatatttttttaactaagggccagatttggtca

SEQ ID NO: 34 - protein, 283 aa, Solanum lycopersicum
  MISC_FEATURE: SlMKS1c
MEKSKFQTSLVVLILLLPYAKSTLSGPKSKKHFVLVHTAGHGAWTWYKSVALMRSSGHNVTALDLGASGINPKQALEIPNFSDYLSPLMEFMASLPADKKVVLVGHSFGGLAISKAMETFPEKISVAVFVTAHMPGPNINVATIYTELFKSISQPDNRIIYDNGPTNPPTTYILGPKYMETDVYQRSPTQDLALASTLVRPINFYSLEDVSKEIVITSKRYGSVWRAFIVAVEDKLLKKEFQHLMIEKNPPDEVKIQGSDTMIMRSKPQQLFTTLVSIANKYT

SEQ ID NO: 35 - DNA, 1970 bp, Solanum habrochaites
  misc_feature: ShMKS1
tttatgtggcatttttctcatttcgagtttcaaacaagtctattttttaccgtaagtttttcatagaccctttaaatattttgaattatcaattattgtgacttgtaatactttttacatagtttacaaatatataaattttatttcaaaaaatgtgaagattccatacgcaaatgtccggtcaaagttaaattgtttgattctcgaaaaacaaaaagtgtcatataaaatgggactgaggagtatttgatatgtaaaaaaattgaaccttataacactttccgtataatttttgtatatctaaattttttgtttaaaatatcgaattaatgtaattttattttaaaaattatgatattttctgtatagttttttaactttttttttttaaaaaaatatcaaagtaatgttatttcacttttttaaaaagatagacaaattaacttttaaaacgcatgacaattaaaatgggctgctagagagtactaaggtaaatacctaaatgtcgttgtaaacacgtaatttacgccaagttaggggtgatcttgagatacttatatgaacatcaaatattcccttcattattatttaattctattcatttttctacctacacaccaaattaaaaaagtcatatatcgtaactaaaaattaaaaatagagtatcccactactctctcttcccttgcacgtcaatcaaacaaacacgtgatttacaccaagttagggcttaggggtgatcatgagatacttatatgcacatcaaatatcatctccatcattgattctaatcatttttccaacatacacatgaatgaaagtgatgtcataattaaaaattacaaattgagattcatttaatatattaagaaaaatcattaaataacatgacaaaattctaatgagaattcatcacatatattgtgcactaaatcccaacaaaaaacaccaattccaaaagaagtgaggtttgttcaaactttgtggtagttgttcaaaaatggagaaaagcatgtcgccatttgttaagaagcactttgtgctagttcatactgcattccacggagcgtggtgctggtacaagattgtggcattgatgagatcttcagggcataatgtcacagctcttgacttgggcgcttcagggatcaaccccaaacaggccctccaaatcccaaatttttctgattacttgagtccgctaatggagttcatggcttcactccctgcaaatgaaaaaataattctcgtaggtcatgccttaggtggactcgccatttctaaagccatggagacctttccagaaaagatttcagttgctgtatttctcagtggtctaatgcctggtccaaatatcgatgcaaccaccgtctgcactaaggtaccaattttccatatatattcacaaccaccgtttcgtttgaaactaattaaataaatatacttcattttaggctggtagtgcagtgctaggtcaactggataattgtgttacatacgaaaatggaccaacgaatcctccaaccactctcatcgcaggtcccaagttcttggcaactaatgtttaccatctgagcccaattgaggtagcaaaaatcataatacaaacataattgcaattgaaatagctcatcactgactgaactaatttaaatcattttttttttaattcttcttggtgttaggatttggcgctggccactgcactagtgaggccactttatttatatctcgcggaagatatttctaaggaggtagttctttcaagcaaaagatatggatccgttaagcgagtgttcattgttgctactgaaaatgatgccttaaagaaagaatttctaaaattgatgattgaaaagaatccacctgatgaagtgaaagagatcgaggggtctgaccacgtgaccatgatgtctaagccccaacaactttttactactcttctaagcatcgctaacaagtataaataa

SEQ ID NO: 36 - DNA, 7062 bp, Solanum lycopersicum
  misc_feature: SlMKS2a
ctcccagtttttctttgctatgtaattggtaccaaaaagtcataaacgaacaccaaattaaggtataattataccaaatacatatttatgtatctggataattatatttttattattttttaaatatatataaatatattaaatacattacaaatttaatgttaaataaatgtatttatacatacacatcatatacattacattaacatatcaatatcctctcgaaaccatatatatagagagagactacactaaatatttagagttcaaacaaaatataaaattatcaaaactatcaatgtataatttatttttatatataataataatatattaatttgtcctttgagatgactttgttggtttggagggtggttattcacaaggatggcccgggatcaatctcccctcaatgttttttaaagttaagcctgtcacataagatttgcctaatacgatttacattcgctttatgatttgcttatctattgcggtttatattctcgcgttgtttgttgaaattcgaaaaaatatatatatatattaatttaactttaactcatttaattgatccaaattaatgtcaatcgttcatttgcccccctatacaaccgtcctagcaagtaggaagcatcacctaaacaacacattttgagctcaccttgtttttgccaatccgctgtcacgagtctttcatgtgctacaaaacaacggcacggaaaatttttggattgttgttacgtgttatttcataatatattatattattattttaataaatattatatttaaaataatttatttgttttgtgtcattagttatatatttatatttgaataataaatttacaaataaaatagaatatataacggaaaataatatttaaattgacaatccacaacgtgttaacgcctatacatatacatacaccaaatttcccaattgttccaatttacgtctattctacctctgcaaattcttcaccattttttagacgcaatgtctcaatgcatcgcttccccgttgattcgcagcattggatccacttcagtcggtaactcactgttgccgaatcatcggccaccgtctacattaccggtcagtcctcaccggcagctcctgcttccaaatttacagttatccgtcagtaaattgaggagttttcgagctcatgcttttgatctcaaaggtagccaagggtatgtctatatatatatatatcttttactccaccaatcccattttatctgaagtatttgattaggcgcggagtttatggataaaaggaagacctaaatcaaccagtatatatgtgtgtatggatatattgtattgttataaatcatctaatgaaatggaaaagtgaaaagtgttattaaatatagaaatgtgatatggttaagtgagacgtttgaagtcaaactgttaccggctgtagaaaggtgtctcaggtgatcttgtaaagtggaaaattgaagttaaattgttatggaatatagaaaggtgtctcaaggtgatctcgtaaaatgggaagttggaaatcaagttgttatcgaatatagaaaggtttctcagggtgatctcgtaaaattggaattttgaagtcaaattgttactgaatataggaaggtgtcatgggtagtaacttacagttccattcaaaattcatcctgtatgacaaaacatagtccggatcatgctttggatgacggatgagggttgtctaggttgtcaatgagggtaaagtaagtctaattatgatcagatactctttaagtattgtattcattggcttgtgtccacttgatttcaactgaatgggcagaggagttatgtagtttgttgtaactagtttgggctttagatatagttgattgattggttttgctgtagcttctgttaggtttgaacttgattagaacctatgttttctccatctgaatgaagggctatgcattttcaatttctacaattggtggaaactgattgattgaataatgttttttttttatcagaattctggaaaaggtttttttttgggaaagaaaaatggaaaaccttttattctttttgtgtcgagcgttttataggcttcccctttcttgtagtttcattttaagtttcagcaagaattggtatttttagtttgctcattgacatagtctattttttcctatttataggagcttaccttttgctcttgctttgcagaatggctgagttccatgaagttgaactcaaagtccgggactatgaattggatcagtatggtgttgtaaacaatgctatttatgcaagttattgccaacatggtaaggtttatggtttcgatctgtacttcagtttacaactaccatattatacatgtgctttcattcatcaaaaagcatataatactgcgcttttcccttttaatgaaaaaggatttactcaagggagaaattttttctggcaactgttatgagtagaaagctagaaattactttttttaaaaaaaaaactgaagtaaactagaaattactggaaaaggatcttttgtatctgttcaacattctttgtaaccctatagttagatcatctgttacccgtgtttatggaatgtgtttctctctcaataacttgagatgatgccacccaaaaatggatgatgaatatgatttcctttgtctgcttattactagaaacatgttgaatcccaagtttgaagggatctgatgtggtcaatgactgtttgaatcttgcatttagacatgctaacgataaagccaatatccactttgtatgtgaactaattgattgccaaatagttgtttgccagaagctcagaacttgctcagttataaatcaataattttaagttaataatatgtctatcctaatgaaaaagaagttaataatttgtctattcaaaatgttgttaagtaattggcacggttcattacctgattacccgtgatatggaatcaaggatatcaaaattcaagtcttcccaacgtaataagatctgttacattgtgagtgacctatgtatacaagttgagtttttttaataaaccaataaaaagtttctgtttaatttctataaatttatatcagatctttctagttcctcgactattattgaagtatactgacaagatgattactttaaaggatttaaattaactctttatctttgtcaagatcaatactttgagggatttgaacttgccttgtaaaaaaaggaattaaactaacagctgcaaagtttcttacgctaaattccaaaaatggggccagtatactactcttattacaaattttggcgtatgagttctacctataatagacaaagttactggtatctgtaggtgaaaaaaaagatcctccttctaaaaagcttagagtaatgagaatttacttgttcataatgctattatatgatcagacaatctggtggattatttggaaagagagaaactagagattttcaaggcaaaaaggagaatgtaaccagtcttgagaattgtacttctttgctttcttttcggagcaatgtggcaaatgaacatgatacttatgatgtggaggccatggttcactttatttagctcactacacagttagtgatgactacctttgatgtgttctcttctctataacaacttgatgtttgttacatttataaaatttcaccttatcaaaaaataaataaattagaatatgatcaggacttttgacatgaaagaacagtaaaaagaaaaataataacagttcagccatccagttaaatagaaactaattagagataacccaatgtcatttttctagaggcaaacaataatatttagataactcaagaacagattgtggaactccaaaaggtgatagtttctttagttgattacttctgtgtagatagagttcgagaaagttttacttccgtgtagttttttctttactgattattttcattttttcaataagtaccctttccaactcaattaagtgaattatttgatggcacattagtgttaaggcaactattgcagctttatagtatttaagtggaagtgtagcaaaaggtggagctaggtttaatcttgcaatgacttgaactccaaatgcgcagaaaggtctgtcctttttcatgatatagtaaaacaaattgatgagtatagagaaaagagatatttttgaaataagctgacatttttctgataatctaggttgttacctcaaggaagtttgtccttttcgataagtagctaattttattgcttcaaaaaacagcactaagcttgtattgcatttgcatgtgtacatgcctacatagtgcattactatacctctgcttcctcagtactatctactgaaaaactaagcaattctatcatatttcctatatcatatacatcatgtctacagtaagaagagaaataaatcataaatgtaaacttgtaaatgctttctgatttgctcttaaaattcttcattcctttctgtccaaaacaccgacgagatgctaactggcactgtgtcacatattcttgtcctatgcaatctcctttgcttttcaatgctgttgtaggttcttcactattttggtatagtctattaaaaatcagtctggtgcactaaagctctcgctatgcacggggtctagggaaggctggaccagaagggtctattgtatgcggtcttaccctgcatttttggaagaggctgtttcaatggcttgtaactgtgacctcccaggtcacatggcagtaacctttctagttatgacaaggctccccttctcttggtattggtatagaattttagtatagtctgttgcatattaaaaatgctcaggaggaacttccatagctgtgaagccattgagaagtgtacaaactagaaacagataatttgcatcctcttcctcctccttgcagagataatatctcccagaaaacatcaatccccttctctgaaatttgtgtcaagttaggctagaagcatgtgcaatatccagattaacactttcttgtgctttggctttgtataatctcctccttagccaaaagggattgtgatgtacttcacacctaagttcactgtgtagagtggtgtccaagttagagaatctggttcatttgattgttgtagttgtccctgttctcgtaactattgagtcattctttccagctcctcatttacgagagggaaaacagtcatcagttacaactgatcaagaaaaaaaagtagcagtagttgtcattaatgaagtgagtcttttcctccatatttttccctttccctaaggagaagtttctatgttgaatcttttgttattctgggattttgctctagcctccttctgtacaaggacgttaccttgttgtatattatcatatactggatatgacattgtccatatcaaaaactttcaaatgacgacaatttaactaatcttgtagttatgacttatttttaataaatgaaacaggtcgccatgagcttctagaaaggattggtataagtgctgatgaagtggcacgcagtggtgacgcactagcactaacagagctgtcacttaagtatctagcacctctaagggtatgaccctcatatctaaacatccttaagaaccaagaaatatgcaaccagaaactttagaccttggttaagtgtcctattcaatttgaattttgtttcacaaaactttgcatttgaatatgaagtttagatcttgggatacatagaaatgaagaataaaatgtttaattgcaagtgtgagaagtttggattagcataattaggaaggttaatgtcaaatggataatggttcggctaaatgaagctttttacagctgattataataatgtgacactgccttctttccaaattacttgggacactgtctttgtttatctataattacttgtcttttctcttcagtaagtataagaaactttactttaccatgaattggaggaactacaatcaaataaagattagtctacattccgttaatctttatttgacttgctttcaattgattatgctacaattaaaactaagctattattttagatatcatctggctctaagttaacaatttgttcaaacaaaccttgtgttctgtactatcagactcagtcatttacttgggacgtgagcttctttcttctgaacaggactggttgatctcttataacttcaaacttgaattgaactgcttgaaatttatgttatcctgcctgttctcattactttcatcattggttcagagtggagatagatttgtcgtgaaggcacgaatatctgattcttcagctgctcgtttgtttttcgaacacttcatcttcaagcttccagatcaagaggtcagttaccactattaccgcgtttttttttttttggaacaaaaccaccttcatatctcaatgtattctgttactacttttttccagcccatcttggaggcaagaggaatagcagtgtggctcaataaaagttaccgtcctgtccgaatcccggcagagttcagatcaaaatttgttcagttccttcgccaggaggcatccaactaatgtgcttgttcaacaaaatccagaagagttcttttgatcaaacatttttctctgaaagtgaaaatttactccttctatatactgacccaaaaaatctagcaacttaaggtatttttgtttggttaatatcactcttggtccctcaatttttaataatattgaatttagtccctgtccctctacagttgggtacacaacacccttccaagcctccgctcgtggagttacatcttgtatagttacatcttgtatgttctaatgttgttgtagcttttgaatttagtccttgtaatatcttatcacttttgatcttcgattaatcgaaatatgtacttttaatcctttttacttgtgagtatttacaaatctacaatgattatcaattgctcaccgtttaattattttacgtgttatgttaaagtttgtattgttgctctatttgaatgttttcaagtataacacagagcacttccataatttggtcacctgaccaatttttg

SEQ ID NO: 37 - DNA, 3098 bp, Solanum lycopersicum
  misc_feature: SlMKS2b
aacaaatatctctccgtccggaattgtttgtcatgttatgcttatcgaaatttaatttgattaattttcaaagataaattagatcatattaatttgatattttaaaaaaaatttaaatattttaaaattacatgaaaaatactatatgttacaatttttgcatattaatatgatgaaaaaaatcattttaaaatgttagtcaaagtttttatagtttgactctaaaaatagaaacaatgacaaacaataccgaacggagggattattgatttctctcatttctcaacgtttcaagtttcaaattttaatcatattattttgcaattgtaaagatttgaataatcattttgtgaattttgaaggaggaatctttttcaaatttcaagtcttgacattaaattttcaattttcgattataatcatttattctttacacaaacgtatggttccattcaaaataactatttttttcgaatatgtgtcggcccgtcctgttctatttcagtccgtctcggtaccatctcgatatatagtggaacaggacggaaccgaatattcatcccagtaattacggtacgcttcatcctggtaccagtcaacccgtcccgttccgatttattccggttcggtggtcccagtccggtgcctagtcttacttatgaatatcgcaatcactatttttttttgttttcatttataattaacttatatgaatttacgtaatttttccatattggcggtaaatgagctgagtgaaatccttataagacttggacaatcctactccgtttgagctaggctgagttaggtctaatatctagtttaacattaaagtttattatagacttttaacctaacctcttcttttttttttgacaatttgctattaagtgcttcgaacgcaataatagaagcataaactttaatgtctacatatacttgaaacaaacttcccaatattttgaatctccctctacaaattcttcacacacaaaaaaaaatgtcacaatccatagtttcccctttgattggcaacaattgccttatctcactgtttccgaatcgtcggccaccatctacatttccggtcaggcaactccatcttccaaatttacagttatcagccagtaaatcgcggagttttgacactaatgcatttgatctcaatggtacacgagggtatgtatatatatatatatctattacatcctctgtcccaattcagatcgcgcaaatatgacaattttgaagtcaaattgttactgaatatagaaaggtgtcattatttgctcgttgacatagtcgattatttatttgtgaactttgcagaataggtgacctatatttccatgaagttgaactcaaagtcagggactatgaattggatcaatttggtgttgtaaacaatgctacttatgcaagttattgtcaacattgtaaggtttactgttttgataatcgatcgtacacaaattacaatatttgaacttaaaaagacttcattttttcaataaatgaaacaggccgtcatgaatatctagaaagaattggcctaagtgttgatgaagtatgtcgcaatggtgatgcattagcaacaacagaaatttcactcaagtatctagcacctctaagggtatgtcgaatttcatcctgtttatgcttcatgtatttgttatatatactacttgttaggttttatttgtcctaaatttcttattagaaaaaaggttttggattgactattcctttttctagtagcaaaaggtttaggactctataaatagagacatgttccttctaacttaatcagcattcacaatgtagtcttaaaggctttgagagttttggtcagagggagaatttgtgggtcacaagcatgataccttatcacttgtgtgaacctcccatgtatttcgaatgaattggttgaggttgtttctctctgtattttgtactatttatagtggattgctcatctcctttgtggacgtaggtcacgttaaatctttgtgtcttttggtatatttctcgttgtgttcttactcgtgatcttgcgaggtttgctttgctagcttccgcgtttacacctgcttattttcggtcctaacactacttggcatgtacttcaagtcgaatttggagtatttaaaatttttggagatacacagaggtgactttattagtcatatgggaaaacagaactgtttagtctttttatggctacaaatgtgaatacaactacttaaaattcaagctatgttatcatttctttgatcattggtttagagtggagatagattcgtcgtgaaggtgagattatccggctctacagctgctcgtttgtatttcgagcatttcatcttcaagcttccagatcaagaggtcagttacgtacatctaattatcattcaattacaaagcgataactttataatactagtgaaatcttactgtatttttcttgaatttacatagcctatcttggaagcaagaggaacatcagtgtggcttgataaaagctaccgtcctgttcgaattccgtcagagttcagatcaaaatttgatcagtttattcatcagaagggatctaattactaatgtgtttgtgaacaaaatgcagcaagagttcttctgaggtgaaaatttgtactatttatacaactaatgtattggtcaatatcacttttggtccctcggtttttaataaattctgaatttagtccttgtgatctagatctacctttactccacatgtaggggtaaggttacgtacgttcagaacatcctcttcatactccacatgtcgagttatattggatatatatgttatcgttctgaatttagtcttgtaatatttaactacatttgatcttctattaattgaacccctttacttgtgagtattttataaagttactatatatgattatcaattgctcactctttgattatttgtgttatatcaagtttgtattgtttgcaccatgtgatcttctctagttcattgacatgtatatacgacctaaatatttatcatattagttcaagggaaaagggtctgatatattcctcaactttgtcatttagagctgatatac

SEQ ID NO: 38 - DNA, 3267 bp, Solanum lycopersicum
  misc_feature: SlMKS2c
ctgtggcaattgttaattggtgggagttggaaaaggaaaaaaaaacattaaaaaagaattgactcccttcaaaattatcttttcctgaatatagaaaataggggaaaatagatatatttttagaaaacactactaattgggttttttatttgctaaaaacttgagattattgtaaaatttgagtcaagttaagcaaattgagttacaatccattattttgttcattttaattaaaataaactttagataaaattaaatattatctcatttatttattcgtccatcttatgccttaatatttacaattaataagttttttaattttaataattcaaaatcttatcattaaatcaatttgatttcatgacccaaaatcttaatcaattcacttaccatcataatccccataaaattttgctagttatattatcaccattttaggccaataaattttattttgcactagtgagtaggaagcattattatttaccaaaataaagacttttgagcaacaaagcctactagctagaggtgtggcattatatggtatataatatatactgaattatcatattatcggaatctttaatatcatagttttaatattattgtacttggtatggtatatgacatatattttggtatttaaaattaacataccaaagtatatagttacatatatattaaaaaaattaaaatattagtattagtataaactaaatgtttaaaagaactttgattaatctgtttcgattgaaatgtaattgattaacaaaaaggagttgtttgtagtatattttgaatatatatttctacatgtgtaatgtgttagtatgatataattgagatctttttaaaattgtcgaaatcataatagtgcctcagtttcacaaagaattcaatagtttgacttgacacaaaatttaaaaaaattaagaagattttgaatcttgtggtcctagattaaacttatgtcaaatgtacaaaattgttctttaatcctgtggtcttaaatatgtcacgtgaaaaactgaaattaaaagagaaacttacataactatactatattaagaaaatatttactatttatagcaataacatttttttcaattgatcacttttaatacatctacaatacatattacaaagaacaatttgttttcaaatataatataatttttattaatgaataatacatttatcacatattttaataaacttataattcaatatgtcaatttcttgccaaataaatataatatatttaaaaaacagttataatgcatatatattgcatacataatccaagtaattttttcaaattaaaatgttaccaaaaaaagaaaaggtcattcttttttaaacatactaaaaaggaaaaaagatcattttttttaacgaaaggagggtgagaactatcttatacgagtatatattaattgagcaaattatactataccataaaatatcaaaaccaaatttcaaaatatcaaattaatttgatatgataatagtataatattttaatactgaaattacgatatcaagactttcaaataccgtatcataccttaccatgcacactcctactacaatctacatatacttgaaacaactaatttcccaaacttgcattttccttctcttctagttctaagatttttcatcaaatgtctcattcgttcagcattgcacccaacctaatgtcgctgaatcatcggtcaccgccgtctacaattccggtcatccctcaccggcaactcccgctcccaaatttacgattatcgtcctgtaaatcgaggggttttgaagcttataatgcgttcgatctcaaaggtacccaacggtacgtgtgtgtatatatatatatatatatatattactctctctgtttagtggcggtacacagaatttttcgttaccttttaaaaaaagtaacaataaataaaacaatgtaacataatattaaaaaaagaacaaaatctcttgtaatttcattttttttctattggtatgtgattttgcagaatgagtgatcaggtctatgaccatgacgttgaactcacagtcagggactatgagttggatcagtttggtgttgtaaataatgctacgtatgtaagttattgtcaacattgtaaggtttactgtttcgataattgatcgtacacaaattacaatatttgacttatttttcaataaatgaaacaggttgtcatgagtttctagaaaaaattggtgttagtgttgatgaagtaacgcgaaatggtgacgcattagcagtaacagagctctcatttaagtttcttgcaccactaagggtatgacgactttcgtcccgtttatgtttcatgtatttgttaagttctgttataccttagttgaatttggagtatttaaaaaatttggagatccaacttcaaatgcctgatataatattgttttgttcagagtggagatagattcgtggtgagggcgcgattatcccactctacagtagctcgattgtttttcgagcatttcatcttcaagcttccagatcaagaggttagttacctctattatcatacaaattacaaagagtcactttatacttgtcaaatcttactgtattttcttaaaattttcacagcctatattggaggcaagaggaatagcagtgtggctcaatagaagttaccgtcctattcgaattccgtcagagttcaattcaaaatttgttaagttccttcaccagaagagttgcggtgtacaacatcgtctctagaccctactcgtagaattacattgttattatttctgaatttagtgcttgtaatgtctaaccacatttgatctttcaattaatttgaaatgtgcaatattaatgtctttacttgtgacttgtcaataataacatgaccatcgttggtaattatgatctttaactttgggtgtacacaagtagacacttaatcttgtataaaattgaacgagtagatacatgtgtcctacgtgacatgatacacataggacaccagataggatacaaaattgtcaaatagggcgccacataggacaaatatgttatttgttcaattttatacaagttttaagtgtttagctgtgcacatctaaataaaaagaggaaatgaagaataaaaaacgcccagcatcatggacaaatttcttggaactcaattatgagtacacgtcaatcccaaacaaagtgaagagattataaatcgactctctttaaagagatgtacacaatagaaagacgaagaa

SEQ ID NO: 39 - DNA, 2775 bp, Solanum habrochaites
  misc_feature: ShMKS2
tggaaaaggaaaaaaaaaacattaaaacgaattgactcccttcaaaattttcttttcctgaataactaattgggtttttaatttgtttaaaaacttgagattattgtaaaatttgagtcaagttaagcaaattgagttacaatccattattttgttcattttaattaaataaactttagataaaattaaatattatctcatttatttattcgtccatcttattccttaatatttacaattaataatttttttaattttaatcattcaaaatcttatcattaaatcaatttgttttgatgacccaaaatcttaatcaattcacttaccatcataatccccatacaattttgctagttatattatcaccattttaggccaataattttattttgtactagtgagtaggaagcattaattatttaccaaaataaagacttttgagcaataaagcctactatctagaggcggcattatatggtatataatatatactgaattatcatattatcgaaatctttaatatcatagtttagtattatggtatttgatatggtatatgacatatattggtatttaaaattaacataccaaagtatatagttagatatatatataaaaaattaaaatattagtattagtataaactaaatgtttagaagttgaaactttgactaatctgtttcgattgaaatgtaattgattaacaaaaaggagttgtttgtagtatattttgaaaatatatttctacatgtgtaatgtgttagtatgatataattgagatcttttttaaaattatcgaaatcataatactccctcagtttcacaaataattatatagtttgacttgacacgaaatttaaaaaaattaagaagatttttgaatcttgtagtcctagattaaacttatgtcaaatgtacaaaattattctttaatcctgtggtcttaaatatgtcacgtggaaaactgaaattaaaagagaaacttacataaatatattatattaagaaaatatttaccatttatagcaataccatttttttcacttgatcacttttaatacatctataatacatattacaaagaacaatttattattcacatataatacaatttttattaatggataatatatttatcacacattttaataaacttataattcaatatgtcaatttcttgccaaataaacataataaatttaaaaaacagttataatacatatatattgcatacataattcaagtaattttttcaaattaaaatgttaccaaaaaagaaaaggtcattcttttttaaacatactaaaaaggaaagaaaatcattttttttaacgagagggtggtgaggactacattatacaagtatatattagttgaacaaattatacatatacttgaaacaacaaattttccaacacttggaatttccttctcttctagttcagaggtttttcatcaaatgtctcattcgttcagcattgcaaccaacatattgttgctgaatcatgggtcaccgccgtctacatttccggtcatccctcaccggcaactcccgctcccaaatttacgattatcgtcccgtaaatcgaggagttttgaagctcatagtgcgttcgatctcaaaagtacccaacggtatgtgtatatatatatatatatatatatatatatattactctctctgtttagtggcggtatacataatttttcattaccttttaaaaaaagtaacaataaataagaaattgttgaagaaacaacaagatctttcttttgtcctggagacctgggacctggcttagtaatgaatgaagggaagtcttcgagctttctccgccagcggcttatgtagtggtcggccttataaagctcgctaagcctcgcttccctctttccttcacttattaagtggaaaatagtcgtcggcattctataagcgacttgacatgtaacataatattaaaaaaaagaacaaaatctcttgtaatttcattttttttttctattggtctgtgactttgcagaatgagtgatcaggtctatcaccatgacgttgaactcacagtcagggactatgagttggatcagtttggtgttgtaaataatgctacttatgcgagttattgtcaacattgtaaggtttactgtttcgataattgatcgtacacaaattacaatatttgaacttaaagacttatttttcaataaatgaaacaggtcgtcatgcgtttctagaaaaaattggtgttagtgttgatgaagtaacgcgaaatggtgatgcattagctgtaacagagctctcacttaagtttctagcaccactaagggtatgacgaatttcgtcctgtttatggttcatgtatttgttagttctgttataccttagtcgaatttggagtatttaaaaaatttggagatccaacttcaaattttatacctgatatacattgttttgttcagagtggagatagattcgtggtgagggcgcgattatcccactttacagtagctcgattgtttttcgagcatttcatcttcaagcttccagatcaagaggttagttgcctctattatcatacaaattacaaagagtcactttatatttgtcaaaacttactgtattttcttcattttttcacagcctatattggaggcaagaggaatagcagtgtggcttaatagaagttatcgtcctattcgaattccgtcagagttcaattcaaaatttgttaaattccttcaccagaagagttgcggtgtacaacatcatctcta

SEQ ID NO: 40 - DNA, 22 bp, Artificial Sequence
  primer
  misc_feature: ShMKS2 forward
gcctatattggaggcaagagga

SEQ ID NO: 41 - DNA, 22 bp, Artificial Sequence
  primer
  misc_feature: ShMKS2 reverse
tgtacaccgcaactcttctggt

SEQ ID NO: 42 - DNA, 22 bp, Artificial Sequence
  primer
  misc_feature: SlMKS2 forward
atgcaagttattgccaacatgg

SEQ ID NO: 43 - DNA, 22 bp, Artificial Sequence
  primer
  misc_feature: SlMKS2 reverse
gaaaaacaaacgagcagctgaa

SEQ ID NO: 44 - DNA, 24 bp, Artificial Sequence
  primer
  misc_feature: ACC forward
ctgctaggaaagctcatcgtatgg

SEQ ID NO: 45 - DNA, 24 bp, Artificial Sequence
  primer
  misc_feature: ACC reverse
gtggtaggaactccagtgataacg

SEQ ID NO: 46 - DNA, 24 bp, Artificial Sequence
  primer
  misc_feature: MaCoA-ACP trans forward
gaatgacggtacgtctagctgttg

SEQ ID NO: 47 - DNA, 24 bp, Artificial Sequence
  primer
  misc_feature: MaCoA-ACP trans reverse
ggtgaagtcacctggctagctaat

SEQ ID NO: 48 - DNA, 22 bp, Artificial Sequence
  primer
  misc_feature: Actin forward
aacaccctgttctcctgactga

SEQ ID NO: 49 - DNA, 22 bp, Artificial Sequence
  primer
  misc_feature: Actin reverse
aacaccatcaccagagtccaac

SEQ ID NO: 50 - DNA, 22 bp, Artificial Sequence
  primer
  misc_feature: forward primer for ORF of MKS2 into TA cloning vector
atgagtgatcaggtctatcacc

SEQ ID NO: 51 - DNA, 20 bp, Artificial Sequence
  primer
  misc_feature: reverse primer for ORF of MKS2 into TA cloning vector
ctcttgatctggaagcttga

SEQ ID NO: 52 - DNA, 25 bp, Artificial Sequence
  primer
  misc_feature: ACP1 forward
tcgccatttgttaagaagcactttg

SEQ ID NO: 53 - DNA, 23 bp, Artificial Sequence
  primer
  misc_feature: ACP1 reverse
tcagacccctcgatctctttcac

SEQ ID NO: 54 - DNA, 25 bp, Artificial Sequence
  primer
  misc_feature: KAS I forward
tcgccatttgttaagaagcactttg

SEQ ID NO: 55 - DNA, 23 bp, Artificial Sequence
  primer
  misc_feature: KAS I reverse
tcagacccctcgatctctttcac

SEQ ID NO: 56 - DNA, 25 bp, Artificial Sequence
  primer
  misc_feature: Acetyl-CoA carboxylase forward
caatgccaatgcttaattattcttc

SEQ ID NO: 57 - DNA, 25 bp, Artificial Sequence
  primer
  misc_feature: Acetyl-CoA carboxylase reverse
tcaagttccaatgagagtaatgttc

SEQ ID NO: 58 - DNA, 20 bp, Artificial Sequence
  primer
  misc_feature: Malonyl-CoA:ACP transacylase forward
atccgcgctcattatgctac

SEQ ID NO: 59 - DNA, 20 bp, Artificial Sequence
  primer
  misc_feature: Malonyl-CoA:ACP transacylase reverse
tgaaagctgggcagagaaat

SEQ ID NO: 60 - DNA, 20 bp, Artificial Sequence
  primer
  misc_feature: 3-Ketoacyl-ACP synthase III forward
tgctgtgaagtttgggtctg

SEQ ID NO: 61 - DNA, 22 bp, Artificial Sequence
  primer
  misc_feature: 3-Ketoacyl-ACP synthase III reverse
tgaggctttgagaggtttcttc

SEQ ID NO: 62 - DNA, 24 bp, Artificial Sequence
  primer
  misc_feature: Enoyl-ACP reductase forward
gagcactatgagtttcaattttgg

SEQ ID NO: 63 - DNA, 20 bp, Artificial Sequence
  primer
  misc_feature: Enoyl-ACP reductase reverse
gaagctatggattggcttcg

SEQ ID NO: 64 - DNA, 20 bp, Artificial Sequence
  primer
  misc_feature: ACP2 forward
aggcacctaaccgtgtatcg

SEQ ID NO: 65 - DNA, 20 bp, Artificial Sequence
  primer
  misc_feature: ACP2 reverse
tggctggattcactctgatg

SEQ ID NO: 66 - DNA, 20 bp, Artificial Sequence
  primer
  misc_feature: MKS1 forward
taagcgagtgttcattgttg

SEQ ID NO: 67 - DNA, 20 bp, Artificial Sequence
  primer
  misc_feature: MKS1 reverse
cgatctctttcacttcatca

SEQ ID NO: 68 - DNA, 20 bp, Artificial Sequence
  primer
  misc_feature: MKS2 forward
tggaggcaagaggaatagca

SEQ ID NO: 69 - DNA, 25 bp, Artificial Sequence
  primer
  misc_feature: MKS2 reverse
caaatgtggttagacattacaagca

SEQ ID NO: 70 - DNA, 19 bp, Artificial Sequence
  primer
  misc_feature: ShMKS2 cDNA forward
atgtctcattcgttcagca

SEQ ID NO: 71 - DNA, 24 bp, Artificial Sequence
  primer
  misc_feature: ShMKS2 cDNA reverse
gagatgatgttgtacaccgcaact

SEQ ID NO: 72 - DNA, 27 bp, Artificial Sequence
  primer
  misc_feature: ShMKS2 promoter forward
ctgtggcaattgttaattggtgggagt

SEQ ID NO: 73 - DNA, 20 bp, Artificial Sequence
  primer
  misc_feature: ShMKS2 promoter reverse
gagcgggagttgccggtgag

SEQ ID NO: 74 - DNA, 21 bp, Artificial Sequence
  primer
  misc_feature: ShMKS1 genomic forward
atggagaaaagcatgtcgcca

SEQ ID NO: 75 - DNA, 30 bp, Artificial Sequence
  primer
  misc_feature: ShMKS1 genomic reverse
tttatacttgttagcgatgcttagaagagt

SEQ ID NO: 76 - protein, 190 aa, Arabidopsis; identical to SEQ ID NO: 10
  MISC_FEATURE: AtMKS2-1

SEQ ID NO: 77 - protein, 189 aa, Arabidopsis; identical to SEQ ID NO: 12
  MISC_FEATURE: AtMKS2-2

SEQ ID NO: 78 - protein, 188 aa, Arabidopsis; identical to SEQ ID NO: 13
  MISC_FEATURE: AtMKS2-3

SEQ ID NO: 79 - protein, 210 aa, Oryza sativa; identical to SEQ ID NO: 14
  MISC_FEATURE: Rice MKS2

SEQ ID NO: 80 - protein, 218 aa, Zea mays
  MISC_FEATURE: Corn MKS2
MHHRFAGLVPTARPALPPIHGGVVGRSYPPVHRSLALRLAPFASASVRRACRPLAVSAQSTSLRPEKFFEVEMKVRDYEIDQYGVVNNAIYASYCQHGRHELLESVGISADAVARSGESLALSELNLKYFAPLRSGDKFVVKVRLAGIKGVRMIFDHIITKLPNHELILEAKATAVCLNKDYYPTRIPRELLSKMQLFLPVDSRGSNEDVNNRNNSCN

SEQ ID NO: 81 - protein, 210 aa, Ricinus communis
  MISC_FEATURE: Castor bean MKS2
MALQQAFIYPMQVTTPLSRANTTWINLHRPSASLLFRVSRPPMSPVVRSLPTVKSCRGLSFLDIRGGKGMNSFVGVELKVRDYELDQYGVVNNAVYASYCQHGRHELLERIGVSADAVARTGDALALSELSLKFLAPLRSGDRFVVKVRISGSSAARLYFDHFIFKLPNEEPILEAKATAVWLDKNYRPVRIPSDMRSKLVQFLKHEESN

SEQ ID NO: 82 - protein, 208 aa, Solanum peruvianum
  MISC_FEATURE: LA1708
MSHSFSILPNLMLLNHRSPPSTIPVIPHRQLPLPNLRLSSCKSRGFEAYNAFDLKGTQRMSDQVYDHDVELTVRDYELDQFGVVNNATYASYCQHCRHEFLEKIGVSVDEVTRNGDALAVTELSLKFLAPLRSGDRFVVRVRLSHSTVARLFFEHFIFKLPDQEPILEARGIAVWLNRSYRPIRIPSEFNSKFVKFLHQKSCGVQHRL